(19) United States
(12) Patent Application Publication
(10) Pub. No.: US 2006/0269515 A1
(43) Pub. Date: Nov. 30, 2006

(54) Interleukin-2 muteins

(75) Inventors: Kimberly Denis-Mize, Concord, CA (US); Carla Heise, Benicia, CA (US); Daniel Menezes, Emeryville, CA (US); Susan E. Wilson, Alameda, CA (US)

Correspondence Address: Chiron Corporation;Intellectual Property - R440, P.O. Box 8097, Emeryville, CA, 94662-8097, US

(73) Assignee: Chiron Corporation, Emeryville, CA (US)

(21) Appl. No.: 11/305,835

(22) Filed: Dec. 16, 2005

Related U.S. Application Data
(63) Continuation of application No. 11/073,251, filed on Mar. 3, 2005, now abandoned.
(60) Provisional application No. 60/550,868, filed on Mar. 5, 2004.
(60) Provisional application No. 60/585,980, filed on Jul. 7, 2004.
(60) Provisional application No. 60/646,095, filed on Jan. 21, 2005.

(51) Int. Cl. A61K 38/20; C07K 14/55; C07K 16/24; C07H 21/04; C12P 21/04

(52) U.S. Cl. 424/85.2; 530/351; 530/388.23; 435/69.52; 435/320.1; 435/325; 536/23.5

(57) ABSTRACT

Novel human interleukin-2 (IL-2) muteins or variants thereof, and nucleic acid molecules and variants thereof are provided. Methods for producing these muteins as well as methods for stimulating the immune system of an animal are also disclosed. In addition, the invention provides recombinant expression vectors comprising the nucleic acid molecules of this invention and host cells into which expression vectors have been introduced. Pharmaceutical compositions are included comprising a therapeutically effective amount of a human IL-2 mutein of the invention and a pharmaceutically acceptable carrier. The IL-2 muteins have lower toxicity than native IL-2 or Proleukin® IL-2, while maintaining or enhancing NK cell-mediated effects, and can be used in pharmaceutical compositions for use in treatment of cancer, and in stimulating the immune response.

This application is a continuation application of U.S. patent application Ser. No. 11/073,251, filed Mar. 3, 2005 from which priority is claimed pursuant to 35 U.S.C. § 120, and claims benefit under 35 U.S.C. § 119(e) of provisional application 60/550,868 filed on Mar. 5, 2004, provisional application 60/585,980 filed on Jul. 7, 2004, and provisional application 60/646,095 filed on Jan. 21, 2005, which applications are hereby incorporated by reference in their entireties.

### FIELD OF THE INVENTION

The invention relates to muteins of human interleukin-2 (IL-2) having improved therapeutic efficacy. Also provided are methods for producing the novel molecules and pharmaceutical formulations that can be utilized to treat cancer and to stimulate the immune system of a mammal.

### BACKGROUND OF THE INVENTION

Interleukin-2 (IL-2) is a potent stimulator of natural killer (NK) and T-cell proliferation and function (Morgan et al. (1976) Science 193:1007-1011). This naturally occurring lymphokine has been shown to have anti-tumor activity against a variety of malignancies either alone or when combined with lymphokine-activated killer (LAK) cells or tumor-infiltrating lymphocytes (TIL) (see, for example, Rosenberg et al. (1987) N. Engl. J. Med. 316:889-897; Rosenberg (1988) Ann. Surg. 208:121-135; Topalian et al. (1988) J. Clin. Oncol. 6:839-853; Rosenberg et al. (1988) N. Engl. J. Med. 319:1676-1680; and Weber et al. (1992) J. Clin. Oncol. 10:33-40). However, high doses of IL-2 used to achieve positive therapeutic results with respect to tumor growth frequently cause severe side effects, including fever and chills, hypotension and capillary leak (vascular leak syndrome or VLS), and neurological changes (see, for example, Duggan et al. (1992) J. Immunotherapy 12:115-122; Gisselbrecht et al. (1994) Blood 83:2081-2085; and Sznol and Parkinson (1994) Blood 83:2020-2022).

Although the precise mechanism underlying IL-2-induced toxicity and VLS is unclear, accumulating data suggests that IL-2-induced natural killer (NK) cells trigger dose-limiting toxicities (DLT) as a consequence of overproduction of pro-inflammatory cytokines including IFN-α, IFN-γ, TNF-α, TNF-β, IL-1β, and IL-6. These cytokines activate monocytes/macrophages and induce nitric oxide production leading to subsequent damage of endothelial cells (Dubinett et al. (1994) Cell Immunol. 157:170-180; Samlowski et al. (1995) J. Immunother. Emphasis Tumor Immunol. 18:166-178). These observations have led others to develop IL-2 muteins that demonstrate preferential selectivity for T cells as opposed to NK cells based on the hypothesis that the high affinity IL-2 receptor (IL-2R) is selectively expressed on T cells (see, for example, BAY50-4798, the N88R IL-2 mutein of mature human IL-2 disclosed in International Publication No. WO 99/60128, and Shanafelt et al. (2000) Nat. Biotechnol. 18:1197-202).

Diverse NK functions such as natural (NK), LAK, and ADCC cytolytic killing, cytokine production, and proliferation depend on the activation of specific intermediates in distinct NK intracellular signaling pathways. Importantly, evidence exists that selective modulation of IL-2-IL-2R interactions can influence diverse downstream NK- and T-cell-mediated effector functions such as proliferation, cytokine production, and cytolytic killing (Sauve et al. (1991) Proc. Natl. Acad. Sci. U.S.A. 88:4636-4640; Heaton et al. (1993) Cancer Res. 53:2597-2602; Eckenberg et al. (2000) J. Exp. Med. 191:529-540).

Proleukin® IL-2 (comprising the recombinant human IL-2 mutein aldesleukin; Chiron Corporation, Emeryville, Calif.) has been approved by the FDA to treat melanoma and renal carcinoma, and is being studied for other clinical indications, including non-Hodgkin's lymphoma, HIV, and breast cancer. However, due to the toxic side effects associated with IL-2, there is a need for a less toxic IL-2 mutein that allows greater therapeutic use of this interleukin. IL-2 muteins that have reduced toxicities and/or enhanced IL-2-mediated NK cell or T cell effector functions would have broader use and would be particularly advantageous for cancer therapy and for modulating the immune response.

### BRIEF SUMMARY OF THE INVENTION

The invention relates to muteins of human interleukin-2 (IL-2) that have improved functional profiles predictive of reduced toxicities. The muteins induce a lower level of pro-inflammatory cytokine production by NK cells while maintaining or increasing NK cell proliferation, maintaining NK-mediated NK, LAK, and ADCC cytolytic functions, and maintain T cell proliferative function as compared to the des-alanyl-1, C125S human IL-2 or C125S human IL-2 muteins. Isolated nucleic acid molecules encoding muteins of human IL-2 and isolated polypeptides comprising these muteins are provided. Clinical uses of these improved human IL-2 muteins in the treatment of cancer and in modulating the immune response are also described.

In one aspect, the invention provides an isolated nucleic acid molecule comprising a nucleotide sequence encoding a mutein of human IL-2. In certain embodiments, the nucleic acid molecule encodes a mutein of human IL-2 comprising an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344.

In certain embodiments, the invention includes an isolated nucleic acid molecule encoding a mutein of human IL-2 comprising a nucleotide sequence selected from the group consisting of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, and 343.

In certain embodiments, the invention includes an isolated nucleic acid molecule comprising a nucleotide sequence encoding a mutein of human IL-2, wherein the mutein has an amino acid sequence comprising residues 2-133 of a sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344.

In certain embodiments, the invention includes an isolated nucleic acid molecule comprising a nucleotide sequence comprising nucleotides 4-399 of a sequence selected from the group consisting of SEQ ID NO: 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, and 343.

In certain embodiments, the nucleic acid molecules described herein may further comprise a substitution, wherein nucleotides 373-375 of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, or 343 are replaced with a triplet codon that encodes alanine.

In certain embodiments, the nucleic acid molecules described herein may further comprise a substitution, wherein nucleotides 373-375 of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, or 343 are replaced with a triplet codon that encodes cysteine.

In certain embodiments, the nucleic acid molecules described herein are further modified to optimize expression. Such nucleic acids comprise a nucleotide sequence, wherein one or more codons encoding the mutein have been optimized for expression in a host cell of interest. Exemplary nucleic acids containing optimized codons may include, but are not limited to, a nucleic acid comprising a nucleotide sequence selected from the group consisting of SEQ ID NO:345, nucleotides 4-399 of SEQ ID NO:345, SEQ ID NO:346, and nucleotides 4-399 of SEQ ID NO:346.

The present invention further includes an expression vector for use in selected host cells, wherein the expression vector comprises one or more of the nucleic acids of the present invention. In such expression vectors, the nucleic acid sequences are operably linked to control elements compatible with expression in the selected host cell. Numerous expression control elements are known to those in the art, including, but not limited to, the following: transcription promoters, transcription enhancer elements, transcription termination signals, polyadenylation sequences, sequences for optimization of initiation of translation, and translation termination sequences. Exemplary transcription promoters include, but are not limited to those derived from polyoma, Adenovirus 2, cytomegalovirus, and Simian Virus 40.

In another aspect, the invention provides cells comprising the expression vectors of the present invention, wherein the nucleic acid sequence (e.g., encoding a mutein of human IL-2) is operably linked to control elements compatible with expression in the selected cell. In one embodiment, such cells are mammalian cells. Exemplary mammalian cells include, but are not limited to, Chinese hamster ovary cells (CHO) or COS cells. Other cells, cell types, tissue types, etc., that may be useful in the practice of the present invention include, but are not limited to, those obtained from the following: insects (e.g., Trichoplusia ni (Tn5) and Sf9), bacteria, yeast, plants, antigen presenting cells (e.g., macrophage, monocytes, dendritic cells, B-cells, T-cells, stem cells, and progenitor cells thereof), primary cells, immortalized cells, tumor-derived cells.

In another aspect, the present invention provides compositions comprising any of the expression vectors and host cells of the present invention for recombinant production of the human IL-2 muteins. Such compositions may include a pharmaceutically acceptable carrier.

In a further aspect, the invention provides an isolated polypeptide comprising a mutein of human IL-2. In certain embodiments, the invention includes an isolated polypeptide comprising an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344.

In certain embodiments, the invention includes an isolated polypeptide comprising amino acid residues 2-133 of an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344.

In certain embodiments, the polypeptides described herein may further comprise a substitution, wherein an alanine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

In certain embodiments, the polypeptides described herein may further comprise a substitution, wherein a cysteine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

In certain embodiments, the isolated polypeptide comprises the amino acid sequence of SEQ ID NO:4 with a serine substituted for cysteine at position 125 of SEQ ID NO:4 and at least one additional amino acid substitution within SEQ ID NO:4, wherein the mutein: 1) maintains or enhances proliferation of natural killer (NK) cells, and 2) induces a decreased level of pro-inflammatory cytokine production by NK cells; as compared with a similar amount of des-alanyl-1, C125S human IL-2 or C125S human IL-2. Exemplary substitutions include, but are not limited to, T7A, T7D, T7R, K8L, K9A, K9D, K9R, K9S, K9V, K9W, T10K, T10N, Q11A, Q11R, Q11T, E15A, H16D, H16E, L19D, L19E, D20E, 124L, K32A, K32W, N33E, P34E, P34R, P34S, P34T, P34V, K35D, K351, K35L, K35M, K35N, K35P, K35Q, K35T, L36A, L36D, L36E, L36F, L36G, L36H, L361, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M461, E61K, E61M, E61R, E62T, E62Y, K64D, K64E, K64G, K64L, K64Q, K64R, P65D, P65E, P65F, P65G, P65H, P651, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L941, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q1261, and Q126V. In certain embodiments, the polypeptides may further comprise a deletion of alanine at position 1 of SEQ ID NO:4.

Increased proliferation of natural killer (NK) cells and decreased levels of pro-inflammatory cytokine production by NK cells can be detected using a NK-92 bioassay. The effects of the polypeptides described herein on proliferation of NK cells and pro-inflammatory cytokine production by NK cells are compared with the effects of a similar amount of des-alanyl-1, C125S human IL-2 or C125S human IL-2 under comparable assay conditions. In certain embodiments, an NK-92 bioassay is used to show that the polypeptides described herein induce a decreased level of the pro-inflammatory cytokine TNF-α relative to that observed for a similar amount of des-alanyl-1, C125S human IL-2 or C125S human IL-2 under comparable assay conditions.

In certain embodiments, a NK3.3 cytotoxicity bioassay is used to show that the polypeptides described herein maintain or improve human NK cell-mediated natural killer cytotoxicity, lymphokine activated killer (LAK) cytotoxicity, or ADCC-mediated cytotoxicity relative to that observed for a similar amount of des-alanyl-1, C125S human IL-2 or C125S human IL-2 under comparable assay conditions.

In certain embodiments, the polypeptides described herein maintain or improve induction of phosphorylated AKT in the NK 3.3 cell line relative to that observed for a similar amount of des-alanyl 1 C125S human IL-2 or C125S human IL-2 under comparable assay conditions.

In certain embodiments, the NK cell proliferation induced by the mutein is greater than 150% of that induced by a similar amount of des-alanyl-1, C125S human IL-2 or C125S human IL-2 under comparable assay conditions.

In certain embodiments, the NK cell proliferation induced by the mutein is greater than 170% of that induced by des-alanyl-1, C 125S human IL-2 or C 125S human IL-2.

In certain embodiments, the NK cell proliferation induced by the mutein is about 200% to about 210% of that induced by des-alanyl-1, C125S human IL-2 or C125S human IL-2.

In certain embodiments, the NK cell proliferation induced by said mutein is increased by at least 10% over that induced by a similar amount of des-alanyl-1, C 125S human IL-2 or C125S human IL-2 under comparable assay conditions.

In certain embodiments, the NK cell proliferation induced by said mutein is increased by at least 15% over that induced by des-alanyl-1, C125S human IL-2 or C125S human IL-2.

In certain embodiments, the pro-inflammatory cytokine production induced by said mutein is less than 100% of that induced by a similar amount of des-alanyl-1, C125S human IL-2 or C125S human IL-2 under similar assay conditions.

In certain embodiments, the pro-inflammatory cytokine production induced by said mutein is less than 70% of that induced by des-alanyl-1, C125S human IL-2 or C125S human IL-2.

In certain embodiments, the invention provides an isolated polypeptide comprising a mutein of human IL-2, wherein the mutein comprises the amino acid sequence set forth in SEQ ID NO:4 with a serine substituted for cysteine at position 125 of SEQ ID NO:4 and at least one additional amino acid substitution within SEQ ID NO:4, wherein the ratio of IL-2-induced NK cell proliferation to IL-2-induced TNF-α production of said mutein is at least 1.5-fold greater than that observed for a similar amount of des-alanyl-1, C125S human IL-2 or C125S human IL-2 under comparable assay conditions, wherein NK cell proliferation at 0.1 nM mutein and TNF-α production at 1.0 nM mutein are assayed using the NK-92 bioassay. In certain embodiments, the ratio is at least 2.5-fold greater than that observed for des-alanyl-1, C125S human IL-2 or C125S human IL-2. In other embodiments, the ratio is at least 3.0-fold greater than that observed for des-alanyl-1, C125S human IL-2 or C125S human IL-2.

In certain embodiments, the invention provides an isolated polypeptide, wherein the mutein demonstrates improved tolerability when administered to a subject as determined by measurement of body temperature using an in vivo temperature chip, measurement of vascular leak, or measurement of maximum tolerated dose in the subject.

In certain embodiments, the invention provides an isolated polypeptide comprising a mutein of human IL-2, wherein the mutein has a higher maximum tolerated dose relative to that observed for des-alanyl-1, C125S human IL-2 or C125S human IL-2, wherein said maximum tolerated dose is determined using a B16F10 melanoma animal model.

In certain embodiments, the invention provides an isolated polypeptide comprising a mutein of human IL-2, wherein said mutein shows comparable or improved anti-tumor activity and reduced adverse effects compared to treatment with des-alanyl-1, C125S human IL-2 or C125S human IL-2 under comparable treatment conditions, wherein said anti-tumor activity is evaluated using a B16F10 melanoma animal model.

In certain embodiments, the invention provides an isolated polypeptide comprising a mutein of human IL-2, wherein said mutein shows comparable or improved anti-tumor activity and reduced adverse effects compared to treatment with des-alanyl-1, C125S human IL-2 or C125S human IL-2 under comparable treatment conditions, wherein said anti-tumor activity is evaluated using a high grade non-Hodgkin's lymphoma Namalwa animal model or a low grade non-Hodgkin's lymphoma Daudi animal model.

In certain embodiments, the invention provides an isolated polypeptide comprising a mutein of human IL-2, wherein said mutein when coadministered with rituximab shows comparable or improved anti-tumor activity and reduced adverse effects compared to treatment with des-alanyl-1, C125S human IL-2 or C125S human IL-2 under comparable treatment conditions, wherein said anti-tumor activity is evaluated using a high grade non-Hodgkin's lymphoma Namalwa animal model or a low grade non-Hodgkin's lymphoma Daudi animal model.

In certain embodiments, the invention provides an isolated polypeptide, wherein the mutein shows improved immune effector cell activation compared with a similar amount of des-alanyl-1, C125S human IL-2 or C125S human IL-2. Activated immune cells may include, but are not limited to, a T cell, a NK cell, a monocyte, a macrophage, and a neutrophil.

In certain embodiments, the invention provides an isolated polypeptide, wherein the mutein shows improved antibody-dependent cellular cytotoxicity (ADCC)-mediated cytolytic killing compared with a similar amount of des-alanyl-1, C125S human IL-2 or C125S human IL-2.

In certain embodiments, the invention provides an isolated polypeptide, wherein the mutein causes less vascular leak as compared with a similar amount of des-alanyl-1, C125S human IL-2 or C125S human IL-2 in an animal model of vascular leak syndrome.

In certain embodiments, the invention provides an isolated polypeptide, wherein the mutein causes less change in body temperature as compared with a similar amount of des-alanyl-1, C125S human IL-2 or C125S human IL-2 in an animal model, wherein body temperature is monitored in the animal with a temperature chip.

In certain embodiments, the invention includes an isolated polypeptide comprising an amino acid sequence for a mutein of human IL-2, wherein the mutein comprises the amino acid sequence set forth in SEQ ID NO:4 with a serine substituted for cysteine at position 125 of SEQ ID NO:4 and with at least one additional amino acid substitution at a position of SEQ ID NO:4 selected from the group consisting of positions 16, 36, 40, 42, 61, 65, 67, 72, 91, 94, 95, and 107. In certain embodiments, the polypeptide further comprises a deletion of alanine at position 1 of SEQ ID NO:4.

In another aspect, the invention provides a method of producing a mutein of human interleukin-2 (IL-2) comprising transforming a host cell with an expression vector comprising any of the nucleic acid molecules described herein and culturing the host cell in a cell culture medium under conditions that allow expression of the nucleic acid molecule as a polypeptide, and isolating the polypeptide. In certain embodiments, the mutein of human interleukin-2 (IL-2) is capable of maintaining or enhancing proliferation of NK cells and also induces a lower level of pro-inflammatory cytokine production by NK cells as compared with a similar amount of a reference human IL-2 mutein selected from des-alanyl-1, C125S human IL-2 and C125 human IL-2, wherein NK cell proliferation and pro-inflammatory cytokine production are assayed under similar assay conditions using the NK-92 bioassay.

In another aspect, the invention provides compositions comprising a therapeutically effective amount of one or more of the polypeptides described herein comprising a mutein of human IL-2. Such compositions may further include a pharmaceutically acceptable carrier.

In another aspect, the invention provides a method for stimulating the immune system of a mammal. The method comprises administering to a mammal a therapeutically effective amount of a human IL-2 mutein that induces a lower level of pro-inflammatory cytokine production by NK cells and maintains or enhances NK cell proliferation compared to a similar amount of a reference IL-2 molecule selected from des-alanyl-1, C125S human IL-2 and C125S human IL-2, wherein NK cell proliferation and pro-inflammatory cytokine production are assayed under comparable assay conditions using the NK-92 bioassay. In certain embodiments, the mammal is a human.

In certain embodiments, the human IL-2 mutein used to stimulate the immune system comprises an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344.

In certain embodiments, the human IL-2 mutein used to stimulate the immune system comprises an amino acid sequence comprising residues 2-133 of an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344.

In certain embodiments, the human IL-2 mutein used to stimulate the immune system may further comprise a substitution, wherein an alanine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

In certain embodiments, the human IL-2 mutein used to stimulate the immune system may further comprise a substitution, wherein a cysteine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

In another aspect, the invention provides a method for treating a cancer in a mammal, comprising administering to said mammal a therapeutically effective amount of a human IL-2 mutein, wherein said mutein induces a lower level of pro-inflammatory cytokine production by NK cells and maintains or enhances NK cell proliferation compared to a similar concentration of a reference IL-2 molecule selected from des-alanyl-1, C125S human IL-2 and C125S human IL-2 under similar assay conditions, wherein said NK cell proliferation and said pro-inflammatory cytokine production are assayed using the NK-92 bioassay. In certain embodiments, the mammal is a human.

In certain embodiments, the human IL-2 mutein used for treating a cancer may comprise an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344.

In certain embodiments, the human IL-2 mutein used for treating a cancer may comprise an amino acid sequence comprising residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

In certain embodiments, the human IL-2 mutein used for treating a cancer may further comprise a substitution, wherein an alanine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

In certain embodiments, the human IL-2 mutein used for treating a cancer may further comprise a substitution, wherein a cysteine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

In another aspect, the invention provides a method for reducing interleukin-2 (IL-2)-induced toxicity symptoms in a subject undergoing IL-2 administration as a treatment protocol. The method of treatment comprises administering IL-2 as an IL-2 mutein.

In certain embodiments, the IL-2 mutein used in treatment comprises an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344.

In certain embodiments, the IL-2 mutein used in treatment comprises residues 2-133 of an amino acid sequence selected from the group consisting of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;

In certain embodiments, the IL-2 mutein used in treatment further comprises a substitution, wherein an alanine residue is substituted for the serine residue at position 125 of SEQ ID NO: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

In certain embodiments, the IL-2 mutein used in treatment further comprises a substitution, wherein a cysteine residue is substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

### BRIEF DESCRIPTION OF THE DRAWINGS

FIG. 1 outlines the schematic for compilation of the combination proliferation/pro-inflammatory cytokine production assay procedure used with IL-2 mutein-stimulated human PBMC isolated from normal human donors.

FIG. 2 shows proliferation and TNF-α production mediated by F42E IL-2 mutein in human PBMC.

FIG. 3 shows proliferation and TNF-α production mediated by L94Y IL-2 mutein in human PBMC.

FIG. 4 shows proliferation and TNF-α production mediated by E95D IL-2 mutein in human PBMC.

FIG. 5 shows proliferation and TNF-α production mediated by E95G IL-2 mutein in human PBMC.

FIG. 6 shows proliferation and TNF-α production mediated by Y107R IL-2 mutein in human PBMC.

FIG. 7 shows maintenance of human NK-mediated LAK and ADCC activity for IL-2 mutein-stimulated human PBMC isolated from normal human donors.

FIG. 8 shows a bar graph comparing the efficacies of Proleukin®, L2-7001®, RL-2, and IL-2 muteins, E95D and Y107R, administered thrice weekly in the B16F10 melanoma lung metastasis model in C57BL/6 mice, as described in Example 11.

FIG. 9 compares mean body weights of mice treated with Proleukin®, L2-7001®, RL-2, or IL-2 muteins, E95D and Y107R, dosed thrice weekly in the B16F10 melanoma lung metastasis model in C57BL/6 mice, as described in Example 11.

FIG. 10 shows a bar graph comparing the efficacies of Proleukin®, L2-7001®, RL-2, and IL-2 muteins, E95D and Y107R, administered according to the “Sleijfer” protocol (5 days on/2 days off/5 days on) in the B16F10 melanoma lung metastasis model in C57BL/6 mice, as described in Example 11.

FIG. 11 compares mean body weights of mice treated with Proleukin®, L2-7001®, RL-2, or IL-2 muteins, E95D and Y107R, dosed according to the “Sleijfer” protocol (5 days on/2 days off/5 days on) in the B16F10 melanoma lung metastasis model in C57BL/6 mice, as described in Example 11.

FIG. 12 shows a bar graph comparing the efficacies of Proleukin®, L2-7001®, RL-2, and IL-2 muteins, F42E and Y107R, administered according to the “Sleijfer” protocol (5 days on/2 days off/5 days on) in the B16F10 melanoma lung metastasis model in C57BL/6 mice, in repeat study as described in Example 11.

FIG. 13 compares mean body weights of mice treated with Proleukin®, L2-7001, RL-2, or IL-2 muteins, F42E and Y107R, dosed according to the “Sleijfer” protocol (5 days on/2 days off/5 days on) in the B16F10 melanoma lung metastasis model in C57BL/6 mice, in repeat study as described in Example 11.

FIG. 14 compares efficacies of Proleukin® and L2-7001®, dosed thrice weekly in the aggressive human Non-Hodgkin's Lymphoma model (Namalwa) in irradiated Balb/c nude mice, as described in Example 12. FIG. 14 shows a plot of the mean tumor volume (mm³) versus time (days post staging).

FIG. 15 compares efficacies of Proleukin®, L2-7001® (, and the Y107R IL-2 mutein dosed thrice weekly in the aggressive human Non-Hodgkin's Lymphoma model (Namalwa) in irradiated Balb/c nude mice, as described in Example 12. FIG. 15 shows a plot of the mean tumor volume (mm³) versus time (days post staging).

FIG. 16 compares efficacies of Proleukin®, L2-7001®, and the E95D IL-2 mutein dosed thrice weekly in the aggressive human Non-Hodgkin's Lymphoma model (Namalwa) in irradiated Balb/c nude mice, as described in Example 12. FIG. 16 shows a plot of the mean tumor volume (mm³) versus time (days post staging).

FIG. 17 compares efficacies of single agent therapy with Proleukin®, L2-7001®, and the Y107R IL-2 mutein dosed thrice weekly in the low grade Daudi human B-cell Non-Hodgkin's Lymphoma model in irradiated Balb/c nude mice, as described in Example 12. FIG. 17 shows a plot of the mean tumor volume (mm³) versus time (days post staging) and a summary of statistical results: % tumor growth inhibition (TGI), partial response/complete response (PR/CR), P value, % body weight (BW) change, and clinical observations.

FIG. 18 compares efficacies of Proleukin®, L2-7001®, and the Y107R IL-2 mutein administered in combination with rituximab thrice weekly in the low grade Daudi human B-cell Non-Hodgkin's Lymphoma model in irradiated Balb/c nude mice, as described in Example 12. FIG. 18 shows a plot of the mean tumor volume (mm³) versus time (days post staging) and a summary of statistical results: % tumor growth inhibition (TGI), partial response/complete response (PR/CR), P value, % body weight (BW) change, and clinical observations.

FIG. 19 compares levels of conditional survival and tumor growth inhibition for mice treated with Proleukin®, L2-7001®, or the Y107R IL-2 mutein in combination with rituximab thrice weekly in the low grade Daudi human B-cell Non-Hodgkin's Lymphoma model in irradiated Balb/c nude mice, as described in Example 12. FIG. 19 shows a plot of the conditional survival (%) versus tumor growth delay time (days for tumor progression to 1000 mm³) and a table summarizing complete response (CR) statistics.

FIG. 20 compares mean body weights of mice treated with Proleukin®, L2-7001®, or the Y107R IL-2 mutein in the presence or absence of rituximab, dosed thrice weekly in the low grade Daudi human B-cell Non-Hodgkin's Lymphoma model in irradiated Balb/c nude mice, as described in Example 12.

FIG. 21 shows a bar graph comparing drug tolerability of Proleukin®, L2-7001®, and the IL-2 muteins, E95D and Y107R, as evaluated in an experimental acute IL-2-induced vascular leak syndrome model in C57B1/6 mice. ¹²⁵I-albumin retention in the lungs of mice, resulting from increased vascular leak caused by treatment with IL-2, was measured as described in Example 13.

FIG. 22 shows a plot depicting the changes in core body temperature of mice in response to treatment with IL-2. Proleukin® and L2-7001® were administered according to the “Sleijfer” protocol (5 days on/2 days off/5 days on) to C57BL/6 mice implanted subcutaneously with a temperature chip to monitor temperature after dosing with IL-2. Temperature was monitored up to 9 hours post-dosing for 10 doses over a 2-week period. The most consistent, significant changes in temperature occurred at 4 hours post dosing on day 5 of treatment.

FIG. 23 shows a plot comparing the core body temperatures of C57BL/6 mice treated with Proleukin®, L2-7001®, or an IL-2 mutein, L94Y, F42E, or E95G. C57BL/6 mice, implanted subcutaneously with a temperature chip, were monitored up to 9 hours post-dosing for 10 doses over a 2-week period as described in Example 14.

FIG. 24 shows a bar graph comparing the core body temperatures of C57BL/6 mice on day 5 at 4 hours post dosing with Proleukin®, L2-7001®, or an IL-2 mutein, E95D, L94Y, Y107R, or F42E. IL-2 was administered according to the “Sleijfer” protocol (5 days on/2 days off/5 days on) to C57BL/6 mice implanted subcutaneously with a temperature chip, as described in Example 14.

FIG. 25 shows the correlation between body temperature decreases and TNF-α plasma levels in C57BL/6 mice treated with Proleukin®, L2-7001®, or an IL-2 mutein, E95D, L94Y, Y107R, or F42E. Bar graphs are shown comparing the changes in body temperature and plasma TNF-α levels of mice on day 5 at 4 hours post dosing with IL-2, according to the “Sleijfer” protocol as described in Example 14. A plot of temperature change versus TNF-α concentration indicates that decreases in temperature and increases in plasma levels of TNF-α are linearly correlated.

### DETAILED DESCRIPTION OF THE INVENTION

The present invention is directed to muteins of human interleukin-2 (hIL-2) that have improved therapeutic efficacy due to their reduced toxicity and/or improved NK or T cell effector functions. The human IL-2 muteins disclosed herein, and biologically active variants thereof, elicit reduced pro-inflammatory cytokine production while maintaining or increasing natural killer (NK) cell proliferation, as compared to the des-alanyl-1, C125S human IL-2 mutein or the C125S human IL-2 mutein. By “pro-inflammatory cytokine” is intended a cytokine that is able to stimulate the immune system. Such cytokines include, but are not limited to, IFN-α, IFN-γ, TNF-α, TNF-β, IL-1β, and IL-6.

The term “mutein” refers to a protein comprising a mutant amino acid sequence that differs from the amino acid sequence for the naturally occurring protein by amino acid deletions, substitutions, or both. The human IL-2 muteins of the present invention comprise an amino acid sequence that differs from the mature human IL-2 sequence by having a serine residue substituted for the cysteine residue at position 125 of the mature human IL-2 sequence (i.e., C125S) and at least one other amino acid substitution, and may further comprise one or more amino acid deletions relative to the mature human IL-2 sequence, such as deletion of the N-terminal alanine (Ala) at position 1 of the mature human IL-2 protein. In alternative embodiments, the human IL-2 muteins of the present invention retain the cysteine residue at position 125 of the mature human IL-2 sequence but have at least one other amino acid substitution, and may further comprise one or more amino acid deletions relative to the mature human IL-2 seuqence, such as deletion of the N-terminal alanin (Ala) at position 1 of the mature human IL-2 protein. These human IL-2 muteins can be glycosylated or unglycosylated depending upon the host expression system used in their production. The particular substitutions disclosed herein result in a human IL-2 variant that retains the desired activities of eliciting reduced pro-inflammatory cytokine production while maintaining or increasing NK cell proliferation, as compared to the des-alanyl-1, C125S human IL-2 mutein or the C125S human IL-2 mutein using the NK-92 cell assays described herein. Having identified the positions within the human IL-2 sequence and the relevant substitutions at these positions that result in an IL-2 variant with reduced toxicity and/or improved NK cell proliferation, it is within the skill of one in the art to vary other residues within the human IL-2 sequence to obtain variants of the human IL-2 muteins disclosed herein that also retain these desired activities. Such variants of the human IL-2 muteins disclosed herein are also intended to be encompassed by the present invention, and are further defined below.

Human IL-2 is initially translated as a precursor polypeptide, shown in SEQ ID NO:2, which is encoded by a nucleotide sequence such as that set forth in SEQ ID NO:1. The precursor polypeptide includes a signal sequence at residues 1-20 of SEQ ID NO:2. The term “mature human IL-2” refers to the amino acid sequence set forth as SEQ ID NO:4, which is encoded by a nucleotide sequence such as that set forth as SEQ ID NO:3. The terms “C125S human IL-2 mutein” or “C125S human IL-2” refer to a mutein of mature human IL-2 that retains the N-terminal alanine residing at position 1 of the mature human IL-2 sequence and which has a substitution of serine for cysteine at position 125 of the mature human IL-2 sequence. C125S human IL-2 mutein has the amino acid sequence set forth in SEQ ID NO:6, which is encoded by a nucleotide sequence such as that set forth as SEQ ID NO:5. The terms “des-alanyl-1, C125S human IL-2” and “des-alanyl-1, serine-125 human IL-2” refer to a mutein of mature human IL-2 that has a substitution of serine for cysteine at amino acid position 125 of the mature human IL-2 sequence and which lacks the N-terminal alanine that resides at position 1 of the mature human IL-2 sequence (i.e., at position 1 of SEQ ID NO:4). Des-alanyl-1, C125S human IL-2 has the amino acid sequence set forth in SEQ ID NO:8, which is encoded by a nucleotide sequence such as that set forth in SEQ ID NO:7. The E. coli recombinantly produced des-alanyl-1, C125S human IL-2 mutein, which is referred to as “aldesleukin,” is available commercially as a formulation that is marketed under the tradename Proleukin® IL-2 (Chiron Corporation, Emeryville, Calif.). For the purposes of the present invention, the des-alanyl-1, C125S human IL-2 and C125S human IL-2 muteins serve as reference IL-2 muteins for determining the desirable activities that are to be exhibited by the human IL-2 muteins of the invention. That is, the desired activity of reduced IL-2-induced pro-inflammatory cytokine production, particularly TNF-α production, by NK cells in a suitable human IL-2 mutein of the invention is measured relative to the amount of pro-inflammatory cytokine production of NK cells that is induced by an equivalent amount of the des-alanyl-1, C125S human IL-2 mutein or C125S human IL-2 mutein under similar assay conditions. Similarly, the desired activity of maintaining or increasing IL-2-induced NK cell proliferation in a suitable human IL-2 mutein of the invention is measured relative to the amount of NK cell proliferation induced by an equivalent amount of the des-alanyl-1, C125S human IL-2 or C125S human IL-2 mutein under similar assay conditions.

Isolated nucleic acid molecules encoding human IL-2 muteins, and biologically active variants thereof, comprising the amino acid sequence of des-alanyl-1, C125S human IL-2 (SEQ ID NO:8) or C125S human IL-2 (SEQ ID NO:6) with at least one other substitution and which induce a lower level of pro-inflammatory cytokine production by NK cells while maintaining or increasing NK cell proliferation, as compared to these two reference IL-2 muteins are provided. The isolated polypeptides encoded by the nucleic acid molecules of the invention are also provided.

Human IL-2 muteins of the invention include the muteins set forth in SEQ ID NOS:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344, which are also referred to herein as “the sequences set forth in even SEQ ID NOS:10-344.” The present invention also provides any nucleotide sequences encoding these muteins, for example, the coding sequences set forth in SEQ ID NOS:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, and 343, respectively. These coding sequences are also referred to herein as “the sequences set forth in odd SEQ ID NOS:9-343.” The muteins set forth in these foregoing amino acid sequences comprise the C125S human IL-2 amino acid sequence with one of the additional substitutions shown in Table 1 below.

In alternative embodiments, the human IL-2 muteins of the present invention have the initial alanine residue at position I of these amino acid sequences deleted, and thus comprise the des-alanyl-1, C125S human IL-2 amino acid sequence with one of the additional substitutions shown in Table 1 below. These muteins thus have an amino acid sequence that comprises residues 2-133 of the sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344. The present invention also provides any nucleotide sequences encoding these muteins, for example, the coding sequences set forth in nucleotides 4-399 of the sequence set forth in SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, or 343.

Biologically active variants of the human IL-2 muteins of the invention, including fragments and truncated forms thereof, that have the desired human IL-2 mutein functional profile as noted herein are also provided. For example, fragments or truncated forms of the disclosed human IL-2 muteins may be generated by removing amino acid residues from the full-length human IL-2 mutein amino acid sequence using recombinant DNA techniques well known in the art and described elsewhere herein. Suitable variants of the human IL-2 muteins of the invention will have biological activities similar to those exhibited by the novel human IL-2 muteins themselves, i.e., they have a low toxicity of the novel human IL-2 mutein (i.e., low or reduced pro-inflammatory cytokine production), as well as the ability to maintain or increase NK cell proliferation, when compared to the reference IL-2 molecule, i.e., des-alanyl-1, C125S or C125S human IL-2, using the bioassays disclosed elsewhere herein. It is recognized that a variant of any given novel human IL-2 mutein identified herein may have a different absolute level of a particular biological activity relative to that observed for the novel human IL-2 mutein of the invention, so long as it retains the desired biological profile of having reduced toxicity, that is, it induces a lower level of pro-inflammatory cytokine production by NK cells, and/or increased NK cell proliferation when compared to the reference human IL-2 mutein.

TABLE 1
Examples of human IL-2 muteins of the invention that comprise
the amino acid sequence of C125S human IL-2 (SEQ ID NO: 6)
or des-alanyl-1, C125S human IL-2 (SEQ ID NO: 8) with at least
one other substitution selected from the group shown below.

| | | | |
|---|---|---|---|
| T7A | L36G | P65E | R81L |
| T7D | L36H | P65F | R81M |
| T7R | L36I | P65G | R81N |
| K8L | L36K | P65H | R81P |
| K9A | L36M | P65I | R81T |
| K9D | L36N | P65K | D84R |
| K9R | L36P | P65L | S87T |
| K9S | L36R | P65N | N88D |
| K9V | L36S | P65Q | N88H |
| K9W | L36W | P65R | N88T |
| T10K | L36Y | P65S | V91A |
| T10N | R38D | P65T | V91D |
| Q11A | R38G | P65V | V91E |
| Q11R | R38N | P65W | V91F |
| Q11T | R38P | P65Y | V91G |
| E15A | R38S | L66A | V91Q |
| H16D | L40D | L66F | V91W |
| H16E | L40G | E67A | V91N |
| L19D | L40N | L72G | L94A |
| L19E | L40S | L72N | L94I |
| D20E | T41E | L72T | L94T |
| I24L | T41G | F78S | L94V |
| K32A | F42A | F78W | L94Y |
| K32W | F42E | H79F | E95D |
| N33E | F42R | H79M | E95G |
| P34E | F42T | H79N | E95M |
| P34R | F42V | H79P | T102S |
| P34S | K43H | H79Q | T102V |
| P34T | F44K | H79S | M104G |
| P34V | M46I | H79V | E106K |
| K35D | E61K | L80E | Y107H |
| K35I | E61M | L80F | Y107K |
| K35L | E61R | L80G | Y107L |
| K35M | E62T | L80K | Y107Q |
| K35N | E62Y | L80N | Y107R |
| K35P | K64D | L80R | Y107T |
| K35Q | K64E | L80T | E116G |
| K35T | K64G | L80V | N119Q |
| L36A | K64L | L80W | T123S |
| L36D | K64Q | L80Y | T123C |
| L36E | K64R | R81E | Q126I |
| L36F | P65D | R81K | Q126V |

Compositions of the invention further comprise vectors and host cells for the recombinant production of the human IL-2 muteins of the invention or biologically active variants thereof. In addition, pharmaceutical compositions comprising a therapeutically effective amount of a human IL-2 mutein disclosed herein or biologically active variant thereof, and a pharmaceutically acceptable carrier, are also provided.

Methods for producing muteins of human IL-2 that induce a lower level of pro-inflammatory production by NK cells and which maintain or increase NK cell proliferation relative to that observed for the reference IL-2 muteins are encompassed by the present invention. These methods comprise transforming a host cell with an expression vector comprising a nucleic acid molecule encoding a novel human IL-2 mutein of the invention, or encoding a biologically active variant thereof, culturing the host cell in a cell culture medium under conditions that allow expression of the encoded polypeptide, and isolating the polypeptide product.

Methods are also provided for stimulating the immune system of an animal, or for treating a cancer in a mammal, comprising administering to the animal a therapeutically effective amount of a human IL-2 mutein of the invention, or biologically active variant thereof, wherein the IL-2 mutein or variant thereof induces a lower level of pro-inflammatory cytokine production by NK cells, and maintains or increases NK cell proliferation compared to des-alanyl-1, C125S human IL-2 or C125S human IL-2 as determined using the bioassays disclosed herein below.

The present invention also provides a method for reducing interleukin-2 (IL-2)-induced toxicity symptoms in a subject undergoing IL-2 administration as a treatment protocol. The method comprises administering an IL-2 mutein of the present invention, i.e., a mutein that induces a lower level of pro-inflammatory cytokine production by NK cells, and which maintains or increases NK cell proliferation compared to des-alanyl-1, C125S human IL-2 or C125S human IL-2 as determined using the bioassays disclosed herein below. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. The invention encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, or 1% (by dry weight) of contaminating protein. When the protein of the invention or biologically active variant thereof is recombinantly produced, preferably culture medium represents less than about 30%, 20%, 10%, 5%, or 1% (by dry weight) of chemical precursors or non-protein-of-interest chemicals.

Biological Activity of Novel Human IL-2 Muteins

The novel human IL-2 muteins of the present invention have an increased therapeutic index compared to the des-alanyl-1, C125S human IL-2 mutein, or compared to the C125S human IL-2 mutein. The latter two muteins are referred to herein as “reference IL-2 muteins,” as the biological profiles of the novel muteins of the invention are compared to the biological profiles of these two previously characterized muteins, where any given comparison is made using similar protein concentrations and comparable assay conditions, in order to classify the muteins of the present invention. The increased therapeutic index of the muteins of the present invention is reflected in an improved toxicity profile (i.e., the mutein induces a lower level of pro-inflammatory cytokine production by NK cells), an increased NK and/or T cell effector function without increased toxicity, or both an improved toxicity profile and an increased NK and/or T cell effector function of these muteins when compared to the toxicity profile and NK and/or T cell effector function of either of these two reference IL-2 muteins.

Three functional endpoints were used to select the muteins with increased therapeutic index: (1) the ability to reduce IL-2-induced pro-inflammatory cytokine production by NK cells as compared to des-alanyl-1, C125S human IL-2 or C125S human IL-2; (2) the ability to maintain or increase IL-2-induced proliferation of NK and T cells without an increase in pro-inflammatory cytokine production by the NK cells as compared to des-alanyl-1, C125S human IL-2 or C125S human IL-2; and (3) the ability to maintain or improve (i.e., increase) NK-mediated cytolytic cell killing as compared to des-alanyl-1, C125S human IL-2 or C125S human IL-2. NK-mediated cytolytic cell killing includes NK-mediated, lymphokine activated killer (LAK)-mediated, and antibody-dependent cellular cytotoxicity (ADCC)-mediated cytolytic killing.

The novel human IL-2 muteins disclosed herein that exhibit the greatest improvements in therapeutic index fall within three functional classes predictive of improved clinical benefit. Of note is that all of these muteins exhibit maintained or increased T cell proliferation activity and NK-mediated cytolytic activity. The first functional class of muteins is characterized by having beneficial mutations that reduce IL-2-induced pro-inflammatory cytokine production by NK cells as compared to a reference IL-2 mutein, i.e., des-alanyl-1, C125S human IL-2 or C125S human IL-2, while maintaining IL-2-induced NK cell proliferation. The second functional class of muteins increases IL-2-induced NK cell proliferation relative to that induced by either of the reference IL-2 muteins, without negatively impacting (i.e., increasing) pro-inflammatory cytokine production relative to that induced by either of the reference IL-2 muteins. The third functional class of muteins includes muteins that are “bi-functional” in that they are able to reduce IL-2-induced pro-inflammatory cytokine production by NK cells while increasing IL-2-induced NK cell proliferation when compared to the levels of these activities induced by either of these two reference IL-2 muteins.

Assays to measure IL-2-induced NK cell proliferation and pro-inflammatory cytokine production by freshly isolated NK cells are well known in the art. See, for example, Perussia (1996) Methods 9:370 and Baume et al. (1992) Eur. J. Immunol. 22:1-6. The NK-92 cell line has phenotypic and functional characteristics of NK cells, including proliferation in the presence of IL-2 (Gong et al. (1994) Leukemia 8:652), however little or no production of TNF-α in the presence of IL-2 has previously been reported (Nagashima et al. (1998) Blood 91:3850). IL-2 bioassays that have been developed for screening functional activities of human NK and T cells are disclosed herein and in the Experimental section below. Though other assays can be used to measure NK cell proliferation and pro-inflammatory cytokine production of NK cells, and T cell effector function, preferably the IL-2 bioassays disclosed herein are used to screen IL-2 muteins of interest to determine whether they retain the desired characteristics of the muteins disclosed herein. Of particular interest is their decreased induction of TNF-α production by NK cells. Thus, in one embodiment, IL-2-induced NK cell proliferation and TNF-α production are measured using the IL-2 bioassay described herein below for the human NK-92 cell line (ATCC CRL-2407, CMCC ID #11925). For a description of the NK-92 cell line, see Gong et al. (1994) Leukemia 8(4):652-658. For purposes of the present invention, this bioassay is referred to as the “NK-92 bioassay.”

By “reduce” or “reduced” pro-inflammatory cytokine production is intended that the human IL-2 muteins of the invention induce a level of pro-inflammatory cytokine production by NK cells that is decreased relative to that induced by the reference IL-2 muteins, i.e., des-alanyl-1, C125S human IL-2 or C125S human IL-2 mutein, particularly with respect to induction of TNF-α production by NK cells. Though the human IL-2 muteins of the present invention induce a minimal level of TNF-α production by NK cells that is at least 20% of that induced by a similar amount of des-alanyl-1, C125S human IL-2 or C125S human IL-2 under comparable assay conditions, the maximal level of TNF-α production by NK cells that can be induced by a mutein of the present invention depends upon the functional class into which a mutein has been categorized.

Thus, for example, in some embodiments, the muteins have been selected for greatly enhanced induction of NK cell proliferation without having a negative impact on IL-2-induced TNF-α production by NK cells (i.e., the second functional class of muteins). In these embodiments, the human IL-2 muteins of the present invention induce a level of TNF-α production by NK cells that is similar to (i.e., ±10%) that induced by the reference IL-2 muteins or, preferably, less than 90% of that induced by the reference IL-2 muteins, where TNF-α production is assayed using the human NK-92 cell line (ATCC CRL-2407, CMCC ID #11925) (i.e., using the NK-92 bioassay disclosed herein) and a 1.0 nM or 100 pM (i.e., 0.1 nM) concentration of the respective human IL-2 muteins. In other embodiments of the invention, the human IL-2 muteins of the present invention induce a level of TNF-α production by NK cells that is less than 90%, preferably less than 85%, even more preferably less than 80% of the TNF-α production induced by a similar amount of des-alanyl-1, C125S human IL-2 or C125S human IL-2 under comparable assay conditions, where TNF-α production is assayed using the human NK-92 cell line (i.e., using the NK-92 bioassay disclosed herein) and a 1.0 nM concentration of the respective human IL-2 muteins. In some embodiments, the human IL-2 muteins of the invention induce at least 20% but less than 60% of the TNF-α production induced by des-alanyl-1, C125S human IL-2 or C125S human IL-2, where TNF-α production is assayed using the human NK-92 cell line (i.e., using the NK-92 bioassay disclosed herein) and a 1.0 nM concentration of the respective human IL-2 muteins. Such muteins, which also maintain or increase IL-2-induced NK cell proliferation relative to the reference IL-2 muteins, fall within the first functional class of IL-2 muteins.

By “maintain” is intended that the human IL-2 muteins of the present invention induce at least 70%, preferably at least 75%, more preferably at least 80%, and most preferably at least 85% and up to and including 100% (i.e., equivalent values) of the desired biological activity relative to the level of activity observed for a similar amount of des-alanyl-1, C125S human IL-2 or C125S human IL-2 under comparable assay conditions. Thus, where the desired biological activity is induction of NK cell proliferation, suitable IL-2 muteins of the invention induce a level of NK cell proliferation that is at least 70%, preferably at least 75%, more preferably at least 80%, and most preferably at least 85%, 90%, 95% and up to and including 100% (±5%) of the NK cell proliferation activity induced by a similar amount of des-alanyl-1, C125S human IL-2 or C125S human IL-2, where NK cell proliferation is assayed under comparable conditions using the same bioassay (i.e., the NK-92 bioassay disclosed herein) and similar amounts of these IL-2 muteins.

By “enhance” or “increase” or “improve” is intended that the human IL-2 mutein induces the desired biological activity at a level that is increased relative to that observed for a similar amount of des-alanyl-1, C125S human IL-2 or C125S human IL-2 under comparable assay conditions. Thus, where the desired biological activity is induction of NK cell proliferation, suitable IL-2 muteins of the invention induce a level of NK cell proliferation that is at least 105%, 110%, 115%, more preferably at least 120%, even more preferably at least 125%, and most preferably at least 130%, 140%, or 150% of the NK cell proliferation activity observed for a similar amount of des-alanyl-1, C125S human IL-2 or C125S human IL-2 using the same NK cell proliferation assay (for example, the NK-92 bioassay disclosed herein).

Assays to measure NK cell proliferation are well known in the art (see, for example, Baume et al. (1992) Eur. J. Immuno. 22:1-6, Gong et al. (1994) Leukemia 8(4):652-658, and the NK-92 bioassay described herein). Preferably NK-92 cells are used to measure IL-2-induced pro-inflammatory cytokine production, particularly TNF-α production, and NK cell proliferation (i.e., the NK-92 bioassay disclosed herein). Suitable concentrations of human IL-2 mutein for use in the NK cell proliferation assay include about 0.005 nM (5 pM) to about 1.0 nM (1000 pM), including 0.005 nM, 0.02 nM, 0.05 nM, 0.1 nM, 0.5 nM, 1.0 nM, and other such values between about 0.005 nM and about 1.0 nM. In preferred embodiments described herein below, the NK cell proliferation assay is carried out using NK-92 cells and a concentration of human IL-2 mutein of about 0.1 nM or about 1.0 nM.

As a result of their reduced induction of pro-inflammatory cytokine production and maintained or enhanced IL-2-induced NK cell proliferation, the human IL-2 muteins of the present invention have a more favorable ratio of IL-2-induced NK cell proliferation:IL-2-induced pro-inflammatory cytokine production by NK cells than does either des-alanyl-1, C125S human IL-2 or C125S human IL-2, where these activities are measured for each mutein using comparable protein concentrations and bioassay conditions. Where the pro-inflammatory cytokine being measured is TNF-α, suitable human IL-2 muteins of the invention have a ratio of IL-2-induced NK cell proliferation at 0.1 nM mutein:IL-2-induced TNF-α production by NK cells at 1.0 nM mutein that is at least 1.5-fold that obtained with des-alanyl-1, C125S human IL-2 or C125S human IL-2 under similar bioassay conditions and protein concentrations, more preferably at least 1.75-fold, 2.0-fold, 2.25-fold, even more preferably at least 2.75-fold, 3.0-fold, or 3.25-fold that obtained with the reference IL-2 muteins. In some embodiments, the human IL-2 muteins of the invention have a ratio of IL-2-induced NK cell proliferation at 0.1 nM mutein: IL-2-induced TNF-α production by NK cells at 1.0 nM mutein that is at least 3.5-fold, 4.0-fold, 4.5-fold, or even 5.0-fold that obtained with des-alanyl-1 human IL-2 or C125S human IL-2 mutein under similar bioassay conditions and protein concentrations.

The muteins of the present invention may also enhance (i.e., increase) NK cell survival relative to that observed with des-alanyl-1, C125S human IL-2 or C125S human IL-2 under similar bioassay conditions and protein concentrations. NK cell survival can be determined using any known assay in the art, including the assays described herein. Thus, for example, NK cell survival in the presence of an IL-2 mutein of interest can be determined by measuring the ability of the IL-2 mutein to block glucocorticosteroid programmed cell death and induce BCL-2 expression in NK cells (see, for example, Armant et al. (1995) Immunology 85:331).

The present invention provides an assay for monitoring IL-2 effects on NK cell survival. Thus, in one embodiment, NK cell survival in the presence of a human IL-2 mutein of interest is determined by measuring the ability of the mutein to induce the cell survival signaling cascade in NK 3.3 cells (CMCC ID#12022; see Kornbluth (1982) J. Immunol. 129(6):2831-2837) using a pAKT ELISA. In this manner, upregulation of AKT phosphorylation in NK cells by an IL-2 mutein of interest is used as an indicator of NK cell survival.

The IL-2 muteins for use in the methods of the present invention will activate and/or expand natural killer (NK) cells to mediate lymphokine activated killer (LAK) activity and antibody-dependent cellular cytotoxicity (ADCC). Resting (unactivated) NK cells mediate spontaneous or natural cytotoxicity against certain cell targets referred to as “NK-cell sensitive” targets, such as the human erythroleukemia K562 cell line. Following activation by IL-2, NK cells acquire LAK activity. Such LAK activity can be assayed, for example, by measuring the ability of IL-2-activated NK cells to kill a broad variety of tumor cells and other “NK-insensitive” targets, such as the Daudi B-cell lymphoma line, that are normally resistant to lysis by resting (i.e., unactivated) NK cells. Similarly, ADCC activity can be assayed by measuring the ability of IL-2-activated NK cells to lyse “LAK-sensitive/NK-insensitive” target cells, such as Daudi B-cell lymphoma line, or other target cells not readily lysed by resting (i.e., unactivated) NK cells in the presence of optimal concentrations of relevant tumor cell specific antibodies. Methods for generating and measuring cytotoxic activity of NK/LAK cells and ADCC are known in the art. See for example, Current Protocols in Immunology: Immunologic Studies in Humans, Supplement 17, Unit 7.7, 7.18, and 7.27 (John Wiley & Sons, Inc., 1996), herein incorporated by reference. In one embodiment, the ADCC activity of the IL-2 muteins of the invention is measured using the NK3.3 cell line, which displays phenotypic and functional characteristics of peripheral blood NK cells. For purposes of the present invention, this assay is referred to herein as the “NK3.3 cytotoxicity bioassay.”

The human IL-2 muteins of the invention may also maintain or enhance IL-2-induced T cell proliferation compared to that observed for des-alanyl-1, C125S human IL-2 or C125S human IL-2 under similar bioassay conditions and protein concentrations. T cell proliferation assays are well known in the art. In one embodiment, the human T-cell line Kit225 (CMCC ID#11234; Hori et al. (1987) Blood 70(4):1069-1072) is used to measure T cell proliferation in accordance with the assay described herein below.

As noted above, the leading human IL-2 mutein candidates identified herein (i.e., those novel muteins having the most improved therapeutic index) fall within three functional classes. The first functional class includes those muteins that induce a lower level of TNF-α production by NK cells, about 60%, or less, of that induced by des-alanyl-1, C125S human IL-2 or C125S human IL-2, when all muteins are assayed under similar conditions at a protein concentration of 1.0 nM, and which maintain or enhance NK cell proliferation relative to des-alanyl-1, C125S human IL-2 or C125S human IL-2. These muteins can be further subdivided into two subclasses: (1) those human IL-2 muteins that enhance (i.e., greater than 100%) IL-2-induced NK cell proliferation relative to that observed for the reference human IL-2 muteins when these muteins are assayed under similar conditions at a protein concentration of about 1.0 nM, but which have reduced (i.e., less than 100%) NK cell proliferative activity relative to that observed for the reference human IL-2 muteins at concentrations of about 0.1 nM or below; and (2) those human IL-2 muteins that enhance (i.e., greater than 100%) or maintain (i.e., at least about 70% up to about 100%) the IL-2-induced NK cell proliferation relative to that observed for the reference human IL-2 muteins when these muteins are assayed under similar conditions at protein concentrations of about 1.0 nM down to about 0.05 nM (i.e., about 50 pM). In one embodiment, IL-2-induced NK proliferation and TNF-α production are determined using NK-92 cells (i.e., using the NK-92 bioassay disclosed herein), in which NK cell proliferation is determined using a commercially available MTT dye-reduction kit (CellTiter 96® Non-Radioactive Cell Proliferation Assay Kit; available from Promega Corp., Madison, Wis.) and a stimulation index is calculated based on a colorimetric readout, and TNF-α is quantified using a commercially available TNF-α ELISA kit (BioSource Cytoscreem™ Human TNF-α ELISA kit; Camarillo, Calif.). Human IL-2 muteins within this first functional class include those muteins comprising the amino acid sequence of des-alanyl-1, C125S human IL-2 (SEQ ID NO:8) or C125S human IL-2 (SEQ ID NO:6) with at least one other substitution selected from the group consisting of F42E, V91D, and L72N, where the residue position (i.e., 42, 91, or 72) is relative to the mature human IL-2 sequence (i.e., relative to SEQ ID NO:4). Muteins of human IL-2 comprising the F42E or V91D substitution in addition to the C125S substitution, which may or may not comprise the N-terminal alanine at position 1 of human IL-2, fall within subclass (1) of this first functional class of muteins. See Example 8, and Table 13 herein below. Muteins of human IL-2 comprising the L72N substitution in addition to the C125S substitution, which may or may not comprise the N-terminal alanine at position 1 of human IL-2, fall within subclass (2) of this first functional class of muteins. See Example 8, and Table 14, herein below.

The second functional class of human IL-2 muteins includes those muteins that strongly increase NK cell proliferation without deleterious impact on IL-2-induced TNF-α production by NK cells. Muteins within this functional group meet three selection criteria: (1) level of IL-2-induced NK cell proliferation that is greater than about 200% of that induced by des-alanyl-1, C125S human IL-2 or C125S human IL-2 at one or more concentrations of human IL-2 mutein selected from the group consisting of 0.005 nM (i.e., 5 pM), 0.02 nM (i.e., 20 pM), 0.05 nM (i.e., 50 pM), 0.1 nM (i.e., 100 pM), or 1.0 nM (i.e., 1000 pM); (2) level of IL-2-induced NK cell proliferation that is greater than about 150% of that induced by des-alanyl-1, C125S human IL-2 or C125S human IL-2 when measured for at least two concentrations of human IL-2 mutein selected from the group consisting of 0.005 nM (i.e., 5 pM), 0.02 nM (i.e., 20 pM), 0.05 nM (i.e., 50 pM), 0.1 nM (i.e., 100 pM), or 1.0 nM (i.e., 1000 pM); and (3) a level of IL-2-induced TNF-α production by NK cells that is similar to (i.e., ±10%) that induced by the reference IL-2 muteins or, preferably, less than 90% of that induced by the reference IL-2 muteins, where TNF-α production is assayed at a mutein concentration of 1.0 nM (i.e., 1000 pM) or 0.1 nM (i.e., 100 pM). In one embodiment, IL-2-induced TNF-α production by NK cells and IL-2-induced NK cell proliferation are determined using NK-92 cells (i.e., using the NK-92 bioassay disclosed herein), in which TNF-α production is measured using ELISA, and NK cell proliferation is measured by an MTT assay as noted herein above. Human IL-2 muteins within this second functional class include those muteins comprising the amino acid sequence of des-alanyl-1, C125S human IL-2 (SEQ ID NO:8) or C125S human IL-2 (SEQ ID NO:6) with at least one other substitution selected from the group consisting of L36D and L40D, where the residue position (i.e., 36 or 40) is relative to the mature human IL-2 sequence (i.e., relative to SEQ ID NO:4). See Example 8, and Table 15 herein below.

The third functional class of human IL-2 muteins includes those muteins that are “bi-functional” in that they induce increased NK cell proliferation and decreased TNF-α production by NK cells relative to the reference IL-2 muteins. Muteins within this third functional class meet the following criteria: (1) induce a level of NK cell proliferation that is at least about 150% of that observed for des-alanyl-1 C125S human IL-2 or C125S human IL-2 when assayed for any one mutein concentration selected from the group consisting of 0.005 nM (i.e., 5 pM), 0.02 nM (i.e., 20 pM), 0.05 nM (i.e., 50 pM), 0.1 nM (i.e., 100 pM), or 1.0 nM (i.e., 1000 pM); and (2) induce a level of TNF-α production by NK cells that is less than about 75% of that induced by des-alanyl-1 C125S human IL-2 or C125S human IL-2 when assayed at a mutein concentration of about 1.0 nM. In one embodiment, IL-2-induced TNF-α production and IL-2-induced NK cell proliferation are determined using NK-92 cells (i.e., the NK-92 bioassay disclosed herein), in which IL-2-induced TNF-α production is measured using ELISA, and IL-2-induced NK cell proliferation is measured by an MTT assay as noted herein above. Human IL-2 muteins within this third functional class include those muteins comprising the amino acid sequence of des-alanyl-1, C125S human IL-2 (SEQ ID NO:8) or C125S human IL-2 (SEQ ID NO:6) with at least one other substitution selected from the group consisting of L19D, F42R, and E61R, where the residue position (i.e., 19, 42, or 61) is relative to the mature human IL-2 sequence (i.e., relative to SEQ ID NO:4). See Example 8, and Table 16 herein below.

The invention also provides human IL-2 muteins meeting other selection criteria that contribute to an improved therapeutic index relative to that observed for des-alanyl-1 C125S human IL-2 or C125S human IL-2. Thus, for example, in another embodiment, the human IL-2 muteins of the invention induce a level of TNF-α production by NK cells that is less than about 100%, preferably less than about 95% or 90%, more preferably less than about 85% of the level of TNF-α production by NK cells that is induced by des-alanyl-1 C125S human IL-2 or C125S human IL-2 when assayed at a mutein concentration of 1.0 nM, and increase IL-2-induced NK cell proliferation to greater than about 130% relative to that induced by des-alanyl-1 C125S human IL-2 or C125S human IL-2 when assayed at a mutein concentration of 0.1 nM. In one embodiment, IL-2-induced TNF-α production and IL-2-induced NK cell proliferation are determined using NK-92 cells (i.e., using the NK-92 bioassay disclosed herein), in which IL-2-induced TNF-α production is measured using ELISA, and IL-2-induced NK cell proliferation is measured by an MTT assay as noted herein above. Human IL-2 muteins with these functional criteria comprise the amino acid sequence of des-alanyl-1, C125S human IL-2 (SEQ ID NO:8) or C125S human IL-2 (SEQ ID NO:6) with at least one other substitution selected from the group consisting of H16D, L19D, L36D, L36P, L40D, L40G, P65L, P65Y, E67A, L72N, L80K, L94Y, E95D, E95G, Y107H, and Y107R, where the residue position (i.e., 16, 19, 36, 40, 65, 67, 72, 80, 94, 95, and 107) is relative to the mature human IL-2 sequence (i.e., relative to SEQ ID NO:4). Muteins meeting these functional criteria also exhibit a ratio of IL-2-induced NK cell proliferation at 0.1 nM mutein:IL-2-induced TNF-α production by NK cells at 1.0 nM mutein that is at least 1.25-fold greater, preferably at least 1.5-fold, 1.75-fold, or 2.0-fold greater, and up to about 2.5-fold to about 2.75-fold greater than that observed for des-alanyl-1, C125S human IL-2 or C125S human IL-2. See also Example 2, and Table 3 herein below, where additional suitable substitutions within the des-alanyl-1, C125S human IL-2 or C125S human IL-2 mutein are listed.

In another embodiment, the human IL-2 muteins of the invention induce a level of TNF-α production by NK cells that is <about 100% of the level of TNF-α production by NK cells that is induced by des-alanyl-1 C125S human IL-2 or C125S human IL-2 when assayed at a mutein concentration of 1.0 nM, and increase IL-2-induced NK cell proliferation to greater than about 150% relative to that induced by des-alanyl-1 C125S human IL-2 or C125S human IL-2 when assayed at a mutein concentration of 0.1 nM. In one embodiment, IL-2-induced TNF-α production and IL-2-induced NK cell proliferation are determined using NK-92 cells (i.e., using the NK-92 bioassay disclosed herein), in which IL-2-induced TNF-α production is measured using ELISA, and IL-2-induced NK cell proliferation is measured by an MTT assay as noted herein above. Human IL-2 muteins with these functional criteria comprise the amino acid sequence of des-alanyl-1, C125S human IL-2 (SEQ ID NO:8) or C125S human IL-2 (SEQ ID NO:6) with at least one other substitution selected from the group consisting of L36G, L36H, L40G, and P65F, where the residue position (i.e., 36, 40, and 65) is relative to the mature human IL-2 sequence (i.e., relative to SEQ ID NO:4). Muteins meeting these functional criteria also exhibit a ratio of IL-2-induced NK cell proliferation at 0.1 nM mutein:IL-2-induced TNF-α production by NK cells at 1.0 nM mutein that is at least 1.5-fold greater, preferably at least 2.0-fold greater, and up to about 2.5-fold greater than that observed for des-alanyl-1, C125S human IL-2 or C125S human IL-2. See Example 2, and Table 4 herein below.

Other human IL-2 muteins of the invention induce a level of TNF-α production by NK cells that is greater than and up to about 110%) the level of TNF-α production by NK cells that is induced by des-alanyl-1 C125S human IL-2 or C125S human IL-2 when assayed at 1.0 nM concentration, and increase IL-2-induced NK cell proliferation to greater than 150% relative to that induced by des-alanyl-1 C125S human IL-2 or C125S human IL-2 when assayed at 0.1 nM. In one embodiment, IL-2-induced TNF-a production and IL-2-induced NK cell proliferation are determined using NK-92 cells (i.e., using the NK-92 bioassay disclosed herein), in which IL-2-induced TNF-α production is measured using ELISA, and IL-2-induced NK cell proliferation is measured by an MTT assay as noted herein above. Human IL-2 muteins with these functional criteria comprise the amino acid sequence of des-alanyl-1, C125S human IL-2 (SEQ ID NO:8) or C125S human IL-2 (SEQ ID NO:6) with at least one other substitution selected from the group consisting of L36R, K64G, K64L, P65E, P65G, P65T, and P65V, where the residue position (i.e., 36, 64, and 65) is relative to the mature human IL-2 sequence (i.e., relative to SEQ ID NO:4). Muteins meeting these functional criteria also exhibit a ratio of IL-2-induced NK cell proliferation at 0.1 nM mutein:IL-2-induced TNF-α production by NK cells at 1.0 nM mutein that is at least 1.5-fold greater, preferably at least 1.75-fold greater, and up to about 2.0-fold to about 2.5-fold greater than that observed for des-alanyl-1, C125S human IL-2 or C125S human IL-2. See Example 2, and Table 4 herein below.

In other embodiments, the human IL-2 muteins of the invention induce a level of TNF-α production by NK cells that is less than about 90%, preferably less than about 80% of the level of TNF-α production by NK cells that is induced by des-alanyl-1 C125S human IL-2 or C125S human IL-2 when assayed at 1.0 nM concentration, and induce NK cell proliferation that is at least 95%, preferably at least 105%, more preferably at least 120% to about 200% of that induced by des-alanyl-1 C125S human IL-2 when assayed at 0.1 nM and at 1.0 nM, or which maintain (i.e., at least 70%, preferably at least 75%, 80%, or 85%, more preferably at least 90% up to about 100%) IL-2-induced NK cell proliferation relative to that induced by the C125S human IL-2 mutein at 0.1 nM. In one embodiment, IL-2-induced TNF-α production and IL-2-induced NK cell proliferation are determined using NK-92 cells (i.e., using the NK-92 bioassay disclosed herein), in which IL-2-induced TNF-α production is measured using ELISA, and IL-2-induced NK cell proliferation is measured by an MTT assay as noted herein above. Human IL-2 muteins with these functional criteria comprise the amino acid sequence of des-alanyl-1, C125S human IL-2 (SEQ ID NO:8) or C125S human IL-2 (SEQ ID NO:6) with at least one other substitution selected from the group consisting of H16D, L19D, L36D, L36P, F42E, F42R, E61R, P65L, P65Y, E67A, L72N, L80V, R81K, N88D, V91D, L94Y, E95D, E95G, Y107H, and Y107R, where the residue position (i.e., 16, 19, 36, 42, 61, 65, 67, 72, 80, 81, 88, 91, 94, 95, or 107) is relative to the mature human IL-2 sequence (i.e., relative to SEQ ID NO:4). Other suitable muteins within this category are shown in Table 5 herein below. Muteins meeting these functional criteria also exhibit a ratio of IL-2-induced NK cell proliferation at 0.1 nM mutein:IL-2-induced TNF-α production by NK cells at 1.0 nM mutein that is at least 1.25-fold greater, preferably at least 1.5-fold greater, 1.75-fold greater, 2.0-fold greater, and up to about 2.5-fold to about 2.75-fold greater than that observed for des-alanyl-1, C125S human IL-2 or C125S human IL-2. See Example 3, and Table 5 herein below.

In alternative embodiments, the IL-2 muteins of the invention induce a level of TNF-α production by NK cells that is less than about 80%, preferably less than about 70% of the level of TNF-α production by NK cells that is induced by des-alanyl-1 C125S human IL-2 or C125S human IL-2 when assayed at 1.0 nM concentration, and induce NK cell proliferation that is at least 80%, preferably at least 90%, 95%, 100%, or 105%, more preferably at least 110% to about 150% of that induced by des-alanyl-1 C125S human IL-2 when assayed at 1.0 nM. In one embodiment, IL-2-induced TNF-α production and IL-2-induced NK cell proliferation are determined using NK-92 cells (i.e., using the NK-92 bioassay disclosed herein), in which IL-2-induced TNF-α production is measured using ELISA, and IL-2-induced NK cell proliferation is measured by an MTT assay as noted herein above. Human IL-2 muteins with these functional criteria comprise the amino acid sequence of des-alanyl-1, C125S human IL-2 (SEQ ID NO:8) or C125S human IL-2 (SEQ ID NO:6) with at least one other substitution selected from the group consisting of F78S, F78W, H79F, H79M, H79N, H79P, H79Q, H79S, H79V, L80E, L80F, L80Y, R81E, R81L, R81N, R81P, R81T, N88H, and Q1261, or at least one other substitution selected from the group consisting of E61M, E62T, E62Y, L80G, L80N, L80R, L80W, D84R, N88T, E95M, Y107L, Y107Q, and Y107T, where the residue position (i.e., 61, 62, 78, 79, 80, 81, 84, 88, 95, or 107) is relative to the mature human IL-2 sequence (i.e., relative to SEQ ID NO:4). See Example 3, and Tables 6 and 7 herein below.

In yet another embodiment, the IL-2 muteins of the invention meet the following functional criteria: (1) induce a level of TNF-α production by NK cells that is less than about 100%, preferably less than about 95%, 90%, or 85%, more preferably less than about 80% or less than about 75% of the level of TNF-α production by NK cells that is induced by des-alanyl-1 C125S human IL-2 or C125S human IL-2 when assayed at 1.0 nM concentration; (2) maintain (about 100%) or increase (about 105% up to about 120%) IL-2-induced NK cell proliferation relative to des-alanyl-1 C125S human IL-2 or C125S human IL-2 when assayed at 0.1 nM and 1.0 nM; and (3) improve NK-mediated cytotoxicity activity to greater than about 140% up to about 160% of that observed for C125S human IL-2 mutein and to greater than about 115% up to about 130% of that observed for des-alanyl-1, C125S human IL-2. In one embodiment, IL-2-induced TNF-α production and IL-2-induced NK cell proliferation are determined using NK-92 cells (i.e., using the NK-92 bioassay disclosed herein), in which IL-2-induced TNF-α production is measured using ELISA, and IL-2-induced NK cell proliferation is measured by an MTT assay as noted herein above; and NK-mediated cytotoxicity activity against K562 cells is measured, for example, using the NK3.3 cell line in the NK3.3 cytotoxicity bioassay disclosed herein. Human IL-2 muteins with these functional criteria comprise the amino acid sequence of des-alanyl-1, C125S human IL-2 (SEQ ID NO:8) or C125S human IL-2 (SEQ ID NO:6) with at least one other substitution selected from the group consisting of P34R, P34T, L36A, L36D, L36P, R38P, F42A, and L80R, where the residue position (i.e., 34, 36, 38, 42, or 80) is relative to the mature human IL-2 sequence (i.e., relative to SEQ ID NO:4). See Example 4, and Table 8 herein below.

In another embodiment, the IL-2 muteins of the invention are selected for their ability to induce lower levels of pro-inflammatory cytokines predictive of improved toxicity, as well as improved NK cell proliferation activity, and improved LAK-mediated cytotoxicity activity. These muteins meet the following functional criteria: (1) induce a level of TNF-α production that is less than 100%, preferably less than 95%, 90%, 85%, or 80% of that induced by des-alanyl-1, C125S human IL-2 or C125S human IL-2 when assayed at 1.0 nM concentration; (2) maintain (about 100%) or enhance (about 105% up to about 140%) IL-2-induced NK cell proliferation relative to des-alanyl-1, C125S human IL-2 or C125S human IL-2 when assayed at 1.0 nM or at 0.1 nM; and (3) improve LAK-mediated cytotoxicity activity to greater than about 105%, preferably greater than about 110%, 115%, or 120%, up to about 140% of that induced by des-alanyl-1, C125S human IL-2 or C125S human IL-2. In one embodiment, IL-2-induced TNF-α production and IL-2-induced NK cell proliferation are determined using NK-92 cells (i.e., using the NK-92 bioassay disclosed herein), in which IL-2-induced TNF-α production is measured using ELISA, and IL-2-induced NK cell proliferation is measured by an MTT assay as noted herein above; and LAK-mediated cytotoxicity activity against Daudi cells is measured using the NK3.3 cell line and the NK3.3 cytotoxicity bioassay disclosed herein. Human IL-2 muteins with these functional criteria comprise the amino acid sequence of des-alanyl-1, C125S human IL-2 (SEQ ID NO:8) or C125S human IL-2 (SEQ ID NO:6) with at least one other substitution selected from the group consisting of L36P, L36R, F42A, L80R, and V91Q, where the residue position (i.e., 36, 42, 80, or 91) is relative to the mature human IL-2 sequence (i.e., relative to SEQ ID NO:4). See Example 5, and Table 9 herein below.

In other embodiments, the IL-2 muteins of the invention are selected for their improved toxicity, improved NK cell proliferation activity, and improved ADCC-mediated cytotoxicity activity. These muteins meet the following functional criteria: (1) induce a level of TNF-α production that is less than 100%, preferably less than 95%, 90%, 85%, or 80% of that induced by des-alanyl-1, C125S human IL-2 or C125S human IL-2 when assayed at 1.0 nM concentration; (2) maintain (at least 90%) or enhance (about 105% up to about 115%) IL-2-induced NK cell proliferation relative to des-alanyl-1, C125S human IL-2 or C125S human IL-2 when assayed at 1.0 nM or at 0.1 nM; and (3) improve ADCC-mediated cytotoxicity activity to greater than about 105%, preferably greater than about 110% or 115%, up to about 120% of that induced by des-alanyl-1, C125S human IL-2 or C125S human IL-2. In one embodiment, IL-2-induced TNF-α production and IL-2-induced NK cell proliferation are determined using NK-92 cells (i.e., using the NK-92 bioassay disclosed herein), in which IL-2-induced TNF-α production is measured using ELISA, and IL-2-induced NK cell proliferation is measured by an MTT assay as noted herein above; and ADCC-mediated cytotoxicity activity against Daudi cells in the presence of antibody, such as Rituxan® (rituximab; IDEC-C2B8; IDEC Pharmaceuticals Corp., San Diego, Calif.) is measured using the NK3.3 cell line and the NK3.3 cytotoxicity bioassay disclosed herein. Human IL-2 muteins with these functional criteria comprise the amino acid sequence of des-alanyl-1, C125S human IL-2 (SEQ ID NO:8) or C125S human IL-2 (SEQ ID NO:6) with at least one other substitution selected from the group consisting of D20E or E67A, where the residue position (i.e., 20 or 67) is relative to the mature human IL-2 sequence (i.e., relative to SEQ ID NO:4). See Example 6, and Table 10 herein below.

In another embodiment, the IL-2 muteins maintain or enhance NK cell survival relative to that observed for the reference IL-2 muteins, as measured by a pAKT ELISA assay using NK 3.3 cells. Human IL-2 muteins with these functional attributes comprise the amino acid sequence of des-alanyl-1, C125S human IL-2 (SEQ ID NO:8) or C125S human IL-2 (SEQ ID NO:6) with at least one other substitution selected from the group consisting of L40D, L40G, L80K, R81K, L94Y, and E95D, where the residue position (i.e., 40, 80, 81, 94, or 95) is relative to the mature human IL-2 sequence (i.e., relative to SEQ ID NO:4). See Example 7, and Table 11 herein below, which shows other suitable muteins that meet these functional criteria.

Biologically Active Variants of Novel Human IL-2 Muteins

The present invention also provides biologically active variants of the novel human IL-2 muteins disclosed herein that also have these improved properties relative to the reference IL-2 molecule, i.e., the biologically active variants induce low or reduced pro-inflammatory cytokine production by NK cells, as well as maintain or increase NK cell proliferation, when compared to the reference IL-2 molecule, i.e., des-alanyl-1 C125S or C125S human IL-2, using the standard bioassays disclosed elsewhere herein. As noted previously, it is recognized that a variant of any given novel human IL-2 mutein identified herein may have a different absolute level of a particular biological activity relative to that observed for the novel human IL-2 mutein of the invention, so long as it has the desired characteristics relative to the reference IL-2 molecules, i.e., reduced toxicity, that is reduced pro-inflammatory cytokine production, and/or increased NK cell proliferation when compared to the reference human IL-2 mutein.

By “variant” is intended substantially similar sequences. Variants of the novel human IL-2 muteins described herein may be derived from naturally occurring (e.g., allelic variants that occur at the IL-2 locus) or recombinantly produced (for example muteins) nucleic acid or amino acid sequences. Polypeptide variants can be fragments of the novel human IL-2 muteins disclosed herein, or they can differ from the novel human IL-2 muteins by having one or more additional amino acid substitutions or deletions, or amino acid insertions, so long as the variant polypeptide retains the particular amino acid substitutions of interest that are present within the novel human IL-2 muteins disclosed herein. Thus, suitable polypeptide variants include those with the C125S substitution corresponding to position 125 of the mature human IL-2 sequence (i.e., SEQ ID NO:4), the second amino acid substitution identified herein as contributing to the improved therapeutic index of the novel human IL-2 muteins of the present invention (i.e., a substitution shown in Table 1 above, preferably a substitution shown in Table 12 below), and which have one or more additional amino acid substitutions or deletions, or amino acid insertions. Thus, for example, where the novel human IL-2 mutein comprises the amino acid sequence of des-alanyl-1, C125S human IL-2 (SEQ ID NO:8) or C125S human IL-2 (SEQ ID NO:6) with at least one other substitution selected from the group shown in Table 1, suitable biologically active variants of these novel human IL-2 muteins will also comprise the C125S substitution as well as the other substitution represented by those mutations shown in Table 1, but can differ from the respective novel human IL-2 mutein in having one or more additional substitutions, insertions, or deletions, so long as the variant polypeptide has the desired characteristics relative to the reference IL-2 molecules (i.e., C125S human IL-2 and des-alanyl-1, C125S human IL-2), and thus has reduced toxicity, that is reduced pro-inflammatory cytokine production, and/or increased NK cell proliferation when compared to the reference human IL-2 mutein. Such variants will have amino acid sequences that are at least 70%, generally at least 75%, 80%, 85%, 90% identical, preferably at least 91%, 92%, 93%, 94%, 95%, 96%, 97%, 98%, 99% identical to the amino acid sequence for the respective novel human IL-2 mutein, for example, the human IL-2 mutein set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344, where percent sequence identity is determined as noted herein below. In other embodiments, the biologically active variants will have amino acid sequences that are at least 70%, generally at least 75%, 80%, 85%, 90% identical, preferably at least 91%, 92%, 93%, 94%, 95%, 96%, 97%, 98%, 99% identical to the amino acid sequence set forth in residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344, where percent sequence identity is determined as noted herein below.

In some embodiments of the invention, biologically active variants of the human IL-2 muteins of the invention have the C125S substitution replaced with another neutral amino acid such as alanine, which does not affect the desired functional characteristics of the human IL-2 mutein. Thus, for example, such variants have an amino acid sequence that comprises an alanine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344. In yet other embodiments, the biologically active variants of the human IL-2 muteins of the invention comprise residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344, with the exception of having an alanine residue substituted for the serine residue at position 125 of these sequences.

In alternative embodiments of the invention, biologically active variants of the human IL-2 muteins of the invention comprise the amino acid sequence of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344, with the exception of having a cysteine residue substituted for the serine residue at position 125 of these sequences. In yet other embodiments, the biologically active variants of the human IL-2 muteins of the invention comprise residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344, with the exception of having a cysteine residue substituted for the serine residue at position 125 of these sequences.

By nucleic acid “variant” is intended a polynucleotide that encodes a novel human IL-2 mutein of the invention but whose nucleotide sequence differs from the novel mutein sequence disclosed herein due to the degeneracy of the genetic code. Codons for the naturally occurring amino acids are well known in the art, including those codons that are most frequently used in particular host organisms used to express recombinant proteins. The nucleotide sequences encoding the IL-2 muteins disclosed herein include those set forth in the accompanying Sequence Listing, as well as nucleotide sequences that differ from the disclosed sequences because of degeneracy in the genetic code.

Thus, for example, where the IL-2 mutein of the invention comprises an alanine residue (i.e., A) substitution, such as in the C125S or des-alanyl C125S mutein comprising the T7A, K9A, Q11A, E15A, K32A, L36A, F42A, L66A, E67A, V91A, or L94A substitution, the nucleotide sequence encoding the substituted alanine residue can be selected from the four universal triplet codons for alanine, i.e., GCA, GCC, GCG, and GCT. Similarly, where the IL-2 mutein of the invention comprises an aspartic acid (i.e., D) substitution, such as in the C125S or des-alanyl C125S mutein comprising the T7D, K9D, H16D, L19D, K35D, L36D, R38D, L40D, K64D, P65D, N88D, V91D, or E95D substitution, the nucleotide sequence encoding the substituted aspartic acid residue can be selected from the two universal triplet codons for aspartic acid, i.e., GAC and GAT. Where the IL-2 mutein of the invention comprises an arginine (i.e., R) substitution, such as in the C125S or des-alanyl C125S mutein comprising the T7R, K9R, Q11R, P34R, L36R, F42R, E61R, K64R, P65R, L80R, D84R, or Y107R substitution, the nucleotide sequence encoding the substituted arginine residue can be selected from the four universal triplet codons for arginine, i.e., CGT, CGC, CGA, and CGG. Similarly, where the IL-2 mutein of the invention comprises a leucine (i.e., L) substitution, such as in the C125S or des-alanyl C125S mutein comprising the K8L, 124L, K35L, K64L, P65L, R81L, or Y107L substitution, the nucleotide sequence encoding the substituted leucine residue can be selected from the six universal triplet codons for leucine, i.e., TTA, TTG, CTT, CTC, CTA, and CTG.

Where the IL-2 mutein of the invention comprises a serine (i.e., S) substitution, such as in the C125S or des-alanyl C125S mutein comprising the K9S, P34S, L36S, R38S, L40S, P65S, F78S, H79S, T102S, or T123S substitution, the nucleotide sequence encoding the substituted serine residue can be selected from the two universal triplet codons for serine, i.e., AGT and AGC. Similarly, where the IL-2 mutein of the invention comprises a valine (i.e., V) substitution, such as in the C125S or des-alanyl C125S mutein comprising the K9V, P34V, F42V, P65V, H79V, L80V, L94V, T102V, or Q126V substitution, the nucleotide sequence encoding the substituted valine residue can be selected from the four universal triplet codons for valine, i.e., GTT, GTC, GTA, and GTG. Where the IL-2 mutein of the invention comprises a lysine (i.e., K) substitution, such as in the C125S or des-alanyl C125S mutein comprising the T10K, L36K, F44K, E61K, P65K, L80K, R81K, E106K, or Y107K substitution, the nucleotide sequence encoding the substituted lysine residue can be selected from the two universal triplet codons for lysine, i.e., AAA and AAG. Similarly, where the IL-2 mutein of the invention comprises an asparagine (i.e., N) substitution, such as in the C125S or des-alanyl C125S mutein comprising the T10N, K35N, L36N, L38N, L40N, P65N, L72N, H79N, L80N, R81N, or V91N substitution, the nucleotide sequence encoding the substituted asparagine residue can be selected from the two universal triplet codons for asparagine, i.e., GAT and GAC.

Where the IL-2 mutein of the invention comprises a threonine (i.e., T) substitution, such as in the C125S or des-alanyl C125S mutein comprising the Q11T, P34T, K35T, F42T, E62T, P65T, L72T, L80T, R81T, S87T, N88T, L94T, or Y107T substitution, the nucleotide sequence encoding the substituted threonine residue can be selected from the four universal triplet codons for threonine, i.e., ACT, ACC, and ACA, ACG. Similarly, where the IL-2 mutein of the invention comprises a glutamic acid (i.e., E) substitution, such as in the C125S or des-alanyl C125S mutein comprising the H16E, L19E, D20E, N33E, P34E, L36E, T41E, F42E, K64E, P65E, L80E, R81E, or V91E substitution, the nucleotide sequence encoding the substituted glutamic acid residue can be selected from the two universal triplet codons for glutamic acid, i.e., GAA and GAG. Where the IL-2 mutein of the invention comprises an isoleucine (i.e., I) substitution, such as in the C125S or des-alanyl C125S mutein comprising the K35I, L36I, M46I, P65I, L94I, or Q126I substitution, the nucleotide sequence encoding the substituted isoleucine residue can be selected from the three universal triplet codons for isoleucine, i.e., ATT, ATC, and ATA. Similarly, where the IL-2 mutein of the invention comprises a proline (i.e., P) substitution, such as in the C125S or des-alanyl C125S mutein comprising the K35P, L36P, R38P, H79P, or R81P substitution, the nucleotide sequence encoding the substituted proline residue can be selected from the four universal triplet codons for proline, i.e., CCT, CCC, CCA, and CCG.

Where the IL-2 mutein of the invention comprises a glutamine (i.e., Q) substitution, such as in the C125S or des-alanyl C125S mutein comprising the K35Q, K64Q, P65Q, H79Q, V91Q, Y107Q, or N119Q substitution, the nucleotide sequence encoding the substituted glutamine residue can be selected from the two universal triplet codons for glutamine, i.e., CAA and CAG. Similarly, where the IL-2 mutein of the invention comprises a phenylalanine (i.e., F) substitution, such as in the C125S or des-alanyl C125S mutein comprising the L36F, P65F, L66F H79F, L80F, or V91F substitution, the nucleotide sequence encoding the substituted phenylalanine residue can be selected from the two universal triplet codons for phenylalanine, i.e., TTT and TTC. Where the IL-2 mutein of the invention comprises a glycine (i.e., G) substitution, such as in the C125S or des-alanyl C125S mutein comprising the L36G, R38G, L40G, T41G, K64G, P65G, L72G, L80G, V91G, E95G, M104G, or E116G substitution, the nucleotide sequence encoding the substituted glycine residue can be selected from the four universal triplet codons for glycine, i.e., GGT, GGC, GGA, and GGG. Similarly, where the IL-2 mutein of the invention comprises a histidine (i.e., H) substitution, such as in the C125S or des-alanyl C125S mutein comprising the L36H, K43H, P65H, N88H, or Y107H substitution, the nucleotide sequence encoding the substituted histidine residue can be selected from the two universal triplet codons for histidine, i.e., CAT and CAC.

Where the IL-2 mutein of the invention comprises a tyrosine (i.e., Y) substitution, such as in the C125S or des-alanyl C125S mutein comprising the L36Y, E62Y, P65Y, L80Y, or L94Y substitution, the nucleotide sequence encoding the substituted tyrosine residue can be selected from the two universal triplet codons for tyrosine, i.e., TAT and TAC. Similarly, where the IL-2 mutein of the invention comprises a cysteine (i.e., C) substitution, such as in the C125S or des-alanyl C125S mutein comprising the T123C substitution, the nucleotide sequence encoding the substituted cysteine residue can be selected from the two universal triplet codons for cysteine, i.e., TGT and TGC.

Though the foregoing list of nucleic acid variants have recited the universal codons that could be utilized to encode the particular residue substitutions identified therein, it is recognized that the present invention encompasses all nucleic acid variants that encode the human IL-2 muteins disclosed herein as a result of degeneracy in the genetic code.

Naturally occurring allelic variants of native human IL-2 can be identified with the use of well-known molecular biology techniques, such as polymerase chain reaction (PCR) and hybridization techniques, and can serve as guidance to the additional mutations that can be introduced into the human IL-2 muteins disclosed herein without impacting the desired therapeutic index of these novel human IL-2 muteins. Variant nucleotide sequences also include muteins derived from synthetically derived nucleotide sequences that have been generated, for example, by site-directed mutagenesis but which still encode the novel IL-2 muteins disclosed herein, as discussed below. Generally, nucleotide sequence variants of the invention will have at least 70%, generally at least 75%, 80%, 85%, 90% sequence identity, preferably at least 91%, 92%, 93%, 94%, 95%, 96%, 97%, 98%, or 99% sequence identity to their respective novel human IL-2 mutein nucleotide sequences, for example, with respect to a novel human IL-2 mutein coding sequence set forth in SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, or 343, where percent sequence identity is determined as noted herein below. In other embodiments, nucleotide sequence variants of the invention will have at least 70%, generally at least 75%, 80%, 85%, 90% sequence identity, preferably at least 91%, 92%, 93%, 94%, 95%, 96%, 97%, 98%, or 99% sequence identity to nucleotides 4-399 of the coding sequence set forth in SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, or 343, where percent sequence identity is determined as noted herein below.

As used herein, the terms “gene” and “recombinant gene” refer to nucleic acid molecules comprising an open reading frame encoding an IL-2 mutein of the invention. As used herein, the phrase “allelic variant” refers to a nucleotide sequence that occurs at an IL-2 locus or to a polypeptide encoded by that nucleotide sequence. Such natural allelic variations can typically result in 1-5% variance in the nucleotide sequence of the IL-2 gene. Any and all such nucleotide variations and resulting amino acid polymorphisms or variations in a IL-2 sequence that are the result of natural allelic variation and that do not alter the functional activity of the novel human IL-2 muteins of the invention are intended to be sequences which can be mutated according to the present invention, and all of the resulting sequences are intended to fall within the scope of the invention.

For example, amino acid sequence variants of the novel human IL-2 muteins disclosed herein can be prepared by making mutations in the cloned DNA sequence encoding the novel IL-2 mutein, so long as the mutation(s) does not alter the additional substitution identified in Table 1. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (2d ed., Cold Spring Harbor Laboratory Press, Plainview, N.Y.); U.S. Pat. No. 4,873,192; and the references cited therein; herein incorporated by reference. Guidance as to appropriate amino acid substitutions that may not affect the desired biological activity of the IL-2 mutein (i.e., reduced pro-inflammatory production by NK cells predictive of reduced toxicity and maintained or increased NK cell proliferation) may be found in the model of Dayhoff et al. (1978) Atlas of Polypeptide Sequence and Structure (Natl. Biomed. Res. Found., Washington, D.C.), herein incorporated by reference.

When designing biologically active variants of a human IL-2 mutein disclosed herein, conservative substitutions, such as exchanging one amino acid with another having similar properties, may be preferred. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). See, for example, Bowie et al. (1990) Science 247: 1306, herein incorporated by reference. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr. Preferably, such substitutions would not be made for conserved cysteine residues, such as the amino terminal contiguous cysteine residues.

Guidance as to regions of the human IL-2 protein that can be altered either via residue substitutions, deletions, or insertions outside of the desired substitutions identified herein can be found in the art. See, for example, the structure/function relationships and/or binding studies discussed in Bazan (1992) Science 257:410-412; McKay (1992) Science 257:412; Theze et al. (1996) Immunol. Today 17:481-486; Buchli and Ciardelli (1993) Biochem. Biophys 307:411-415; Collins et al. (1988) Proc. Natl. Acad. Sci. USA 85:7709-7713; Kuziel et al. (1993) J. Immunol. 150:5731; Eckenberg et al. (1997) Cytokine 9:488-498; the contents of which are herein incorporated by reference in their entirety.

In constructing variants of a novel human IL-2 mutein of the invention, modifications to the nucleotide sequences encoding the variants will be made such that variant polypeptides may continue to possess the desired activity. Obviously, any mutations made in the DNA encoding a variant polypeptide must not place the sequence out of reading frame and preferably will not create complementary regions that could produce secondary mRNA structure. A variant of a polypeptide may differ by as few as 1 to 15 amino acid residues, such as 6-10, as few as 5, as few as 4, 3, 2, or even 1 amino acid residue. A variant of a nucleotide sequence may differ by as few as 1 to 30 nucleotides, such as 6 to 25, as few as 5, as few as 4, 3, 2, or even 1 nucleotide.

Biologically active variants of the human IL-2 muteins of the invention include fragments of these muteins. By “fragment” is intended a portion of the coding nucleotide sequence or a portion of the amino acid sequence. With respect to coding sequences, fragments of a human IL-2 mutein nucleotide sequence may encode mutein fragments that retain the desired biological activity of the novel human IL-2 mutein. A fragment of a novel human IL-2 mutein disclosed herein may be 15, 20, 25, 30, 35, 40, 45, 50, 55, 60, 65, 70, 75, 80, 85, 90, 95, 100, 105, 110, 115, 120, 125, 130 amino acids or up to the full length of the novel human IL-2 polypeptide. Fragments of a coding nucleotide sequence may range from at least 45, 60, 75, 90, 105, 120, 135, 150, 165, 180, 195, 210, 225, 240, 255, 270, 285, 300, 315, 330, 345, 360, 375, 390, nucleotides, and up to the entire nucleotide sequence encoding the novel human IL-2 mutein.

The human IL-2 muteins disclosed herein and biologically active variants thereof may be modified further so long as they have the desired characteristics relative to the reference IL-2 molecules, i.e., reduced toxicity and/or increased NK cell proliferation relative to the C125S human IL-2 or des-alanyl-1, C125S human IL-2 mutein. Further modifications include, but are not limited to, phosphorylation, substitution of non-natural amino acid analogues, and the like. Modifications to IL-2 muteins that may lead to prolonged in vivo exposure, and hence increase efficacy of the IL-2 mutein pharmaceutical formulations, include glycosylation or PEGylation of the protein molecule. Glycosylation of proteins not natively glycosylated is usually performed by insertion of N-linked glycosylation sites into the molecule. This approach can be used to prolong half-life of proteins such as IL-2 muteins. In addition, this approach can be used to shield immunogenic epitopes, increase protein solubility, reduce aggregation, and increase expression and purification yields.

Once the variants of the human IL-2 muteins disclosed herein are obtained, the deletions, insertions, and substitutions of the human IL-2 mutein sequences are not expected to produce radical changes in the characteristics of the particular human IL-2 mutein. However, when it is difficult to predict the exact effect of the substitution, deletion, or insertion in advance of doing so, one skilled in the art will appreciate that the effect will be evaluated by routine screening assays. That is, the IL-2-induced NK or T cell proliferation activity can be evaluated by standard cell proliferation assays known to those skilled in the art, including the assays described herein. IL-2-induced pro-inflammatory cytokine production may be measured using cytokine-specific ELISAs, for example, the TNF-α specific ELISA noted elsewhere herein. NK cell survival signaling may be measured by a pAKT ELISA (see, for example, the assay described herein below). NK cell-mediated cytolytic activity (i.e., cytotoxicity) may be measured by assays known in the art (for example, measurement of NK-mediated, LAK-mediated, or ADCC-mediated cytolytic activity as noted elsewhere herein).

The human IL-2 muteins disclosed herein, and biologically active variants thereof, can be constructed as IL-2 fusions or conjugates comprising the IL-2 mutein (or biologically active variant thereof as defined herein) fused to a second protein or covalently conjugated to polyproline or a water-soluble polymer to reduce dosing frequencies or to further improve IL-2 tolerability. For example, the human IL-2 mutein (or biologically active variant thereof as defined herein) can be fused to human albumin or an albumin fragment using methods known in the art (see, for example, WO 01/79258). Alternatively, the human IL-2 mutein (or biologically active variant thereof as defined herein) can be covalently conjugated to polyproline or polyethylene glycol homopolymers and polyoxyethylated polyols, wherein the homopolymer is unsubstituted or substituted at one end with an alkyl group and the poplyol is unsubstituted, using methods known in the art (see, for example, U.S. Pat. Nos. 4,766,106, 5,206,344, and 4,894,226).

By “sequence identity” is intended the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Polypeptide Sequence and Structure 5:Suppl. 3 (National Biomedical Research Foundation, Washington, D.C.). With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are well known in the art.

The determination of percent identity between two sequences can be accomplished using a mathematical algorithm. For purposes of the present invention, percent sequence identity of an amino acid sequence is determined using the Smith-Waterman homology search algorithm using an affine 6 gap search with a gap open penalty of 12 and a gap extension penalty of 2, BLOSUM matrix 62. The Smith-Waterman homology search algorithm is taught in Smith and Waterman (1981) Adv. Appl. Math 2:482-489, herein incorporated by reference. Alternatively, percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

It is further recognized that when considering percentage of amino acid identity, some amino acid positions may differ as a result of conservative amino acid substitutions, which do not affect properties of polynucleotide function. In these instances, percent sequence identity may be adjusted upwards to account for the similarity in conservatively substituted amino acids. Such adjustments are well known in the art. See, for example, Meyers et al. (1988) Computer Applic. Biol. Sci. 4:11-17.

Recombinant Expression Vectors and Host Cells

Generally, the human IL-2 muteins of the invention will be expressed from vectors, preferably expression vectors. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of coding sequences to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses).

The expression constructs or vectors of the invention comprise a nucleic acid molecule encoding a human IL-2 mutein of the present invention in a form suitable for expression of the nucleic acid molecule in a host cell. The coding sequence of interest can be prepared by recombinant DNA techniques as described, for example, by Taniguchi et al. (1983) Nature 302:305-310 and Devos (1983) Nucleic Acids Research 11:4307-4323 or using mutationally altered IL-2 as described by Wang et al. (1984) Science 224:1431-1433. It is recognized that the coding sequences set forth in odd SEQ ID NOS:9-343 begin with a codon for the first residue of the mature human IL-2 sequence of SEQ ID NO:4 (i.e., a codon for the alanine at position 1), rather than a codon for methionine, which generally is the translation initiation codon ATG in messenger RNA. These disclosed nucleotide sequences also lack a translation termination codon following the nucleotide at position 399 of odd SEQ ID NOS:9-343. Where these sequences, or sequences comprising nucleotides 4-399 of odd SEQ ID NOS:9-343, are to be used to express the human IL-2 muteins of the invention, it is recognized that the expression construct comprising these human IL-2 mutein coding sequences will further comprise a translation initiation codon, for example, an ATG codon, upstream and in proper reading frame with the human IL-2 mutein coding sequence. The translation initiation codon can be provided at an upstream location from the initial codon of the human IL-2 mutein coding sequence by utilizing a translation initiation codon, for example ATG, that is already in a sequence that comprises the human IL-2 mutein coding sequence, or can otherwise be provided from an extraneous source such as the plasmid to be used for expression, providing that the translation initiation codon first appearing before the initial codon in the human IL-2 mutein coding sequence is in proper reading frame with the initial codon in the human IL-2 mutein coding sequence. Similarly, the human IL-2 mutein coding sequence disclosed herein will be followed by one or more translation termination codons, for example, TGA, to allow for production of a human IL-2 mutein that ends with the last amino acid of the sequence set forth in even SEQ ID NOS:10-344.

The recombinant expression vectors include one or more regulatory sequences, selected on the basis of the host cells to be used for expression, operably linked to the nucleic acid sequence to be expressed. “Operably linked” is intended to mean that the nucleotide sequence of interest (i.e., a sequence encoding a human IL-2 mutein of the present invention) is linked to the regulatory sequence(s) in a manner that allows for expression of the nucleotide sequence (e.g., in an in vitro transcription/translation system or in a host cell when the vector is introduced into the host cell). “Regulatory sequences” include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression constructs of the invention can be introduced into host cells to thereby produce the human IL-2 muteins disclosed herein or to produce biologically active variants thereof.

The expression constructs or vectors of the invention can be designed for expression of the human IL-2 mutein or variant thereof in prokaryotic or eukaryotic host cells. Expression of proteins in prokaryotes is most often carried out in Escherichia coli with vectors containing constitutive or inducible promoters. Strategies to maximize recombinant protein expression in E. coli can be found, for example, in Gottesman (1990) in Gene Expression Technology: Methods in Enzymology 185 (Academic Press, San Diego, Calif.), pp. 119-128 and Wada et al. (1992) Nucleic Acids Res. 20:2111-2118. Processes for growing, harvesting, disrupting, or extracting the human IL-2 mutein or variant thereof from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

The recombinant human IL-2 muteins or biologically active variants thereof can also be made in eukaryotes, such as yeast or human cells. Suitable eukaryotic host cells include insect cells (examples of Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerenvisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.).

The sequences encoding the human IL-2 muteins of the present invention can be optimized for expression in the host cell of interest. The G-C content of the sequence may be adjusted to levels average for a given cellular host, as calculated by reference to known genes expressed in the host cell. Methods for codon optimization are well known in the art. Individual codons can be optimized, for example, the codons where residue substitutions have been made, for example, the C125S substitution, the C125A substitution, and/or the additional substitution indicated in Table 1. Alternatively, other codons within the human IL-2 mutein coding sequence can be optimized to enhance expression in the host cell, such that 1%, 5%, 10%, 25%, 50%, 75%, or up to 100% of the codons within the coding sequence have been optimized for expression in a particular host cell. See, for example, the human IL-2 mutein sequences disclosed in SEQ ID NOS:345 and 346, where the codons for the E61R and Y107R substitutions, respectively, have been optimized for expression in E. coli.

The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, particle gun, or electroporation. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals.

Prokaryotic and eukaryotic cells used to produce the IL-2 muteins of this invention and biologically active variants thereof are cultured in suitable media, as described generally in Sambrook et al. (1989) Molecular Cloning. A Laboratory Manual (2d ed., Cold Spring Harbor Laboratory Press, Plainview, N.Y.).

Pharmaceutical Compositions

After the human IL-2 muteins or variants thereof are produced and purified, they may be incorporated into a pharmaceutical composition for application in human and veterinary therapeutics, such as cancer therapy or prevention, immunotherapy, and the treatment or prevention of infectious diseases. Thus, the human IL-2 muteins or biologically active variants thereof can be formulated as pharmaceutical formulations for a variety of therapeutic uses. As a composition, the human IL-2 muteins or biologically active variants thereof are parenterally administered to the subject by methods known in the art. Subjects include mammals, e.g., primates, humans, dogs, cattle, horses, etc. These pharmaceutical compositions may contain other compounds that increase the effectiveness or promote the desirable qualities of the human IL-2 muteins of the invention. The pharmaceutical compositions must be safe for administration via the route that is chosen, they must be sterile, retain bioactivity, and they must stably solubilize the human IL-2 mutein or biologically active variant thereof. Depending upon the formulation process, the IL-2 mutein pharmaceutical compositions of the invention can be stored in liquid form either ambient, refrigerated, or frozen, or prepared in the dried form, such as a lyophilized powder, which can be reconstituted into the liquid solution, suspension, or emulsion before administration by any of various methods including oral or parenteral routes of administration.

Such pharmaceutical compositions typically comprise at least one human IL-2 mutein, biologically active variant thereof, or a combination thereof, and a pharmaceutically acceptable carrier. Methods for formulating the human IL-2 muteins of the invention for pharmaceutical administration are known to those of skill in the art. See, for example, Gennaro (ed.) (1995) Remington: The Science and Practice of Pharmacy (19^{th} ed., Mack Publishing Company, Easton, Pa.).

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the human IL-2 mutein pharmaceutical formulations of the invention. Supplementary active compounds can also be incorporated into the compositions.

An IL-2 mutein pharmaceutical composition comprising a human IL-2 mutein of the invention or variant thereof is formulated to be compatible with its intended route of administration. The route of administration will vary depending on the desired outcome. The IL-2 mutein pharmaceutical composition can be administered by bolus dose, continuous infusion, or constant infusion (infusion for a short period of time, i.e. 1-6 hours). The IL-2 mutein pharmaceutical composition can be administered orally, intranasally, parenterally, including intravenously, subcutaneously, intraperitoneally, intramuscularly, etc., by intradermal, transdermal (topical), transmucosal, and rectal administration, or by pulmonary inhalation.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; surfactants such as polysorbate 80; SDS; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. Where formation of protein aggregates is minimized in the formulation process, suitable carriers for intravenous administration include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a protein or antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The human IL-2 muteins of the present invention, or biologically active variants thereof, can be formulated using any known formulation process known in the art for human IL-2. Suitable formulations that are useful in the present method are shown in various patents and publications. For example, U.S. Pat. No. 4,604,377 shows a preferred IL-2 formulation that has a therapeutic amount of IL-2, which is substantially free from non-IL-2 protein and endotoxin, a physiologically acceptable water-soluble carrier, and a sufficient amount of a surface active agent to solubilize the IL-2, such as sodium dodecyl sulfate. Other ingredients can be included, such as sugars. U.S. Pat. No. 4,766,106 shows formulations including polyethylene glycol (PEG) modified IL-2. European patent application, Publication No. 268,110, shows IL-2 formulated with various non-ionic surfactants selected from the group consisting of polyoxyethylene sorbitan fatty acid esters (Tween-80), polyethylene glycol monostearate, and octylphenoxy polyethoxy ethanol compounds (Triton X405). U.S. Pat. No. 4,992,271 discloses IL-2 formulations comprising human serum albumin and U.S. Pat. No. 5,078,997 discloses IL-2 formulations comprising human serum albumin and amino acids. U.S. Pat. No. 6,525,102 discloses IL-2 formulations comprising an amino acid base, which serves as the primary stabilizing agent of the polypeptide, and an acid and/or its salt form to buffer the solution within an acceptable pH range for stability of the polypeptide. Copending U.S. patent application Ser. No. 10/408,648 discloses IL-2 formulations suitable for pulmonary delivery. All of the above patents and patent applications are hereby incorporated by reference in their entireties.

Therapeutic Uses

Pharmaceutical formulations comprising the human IL-2 muteins of the present invention or biologically active variants thereof obtained from these human IL-2 muteins are useful in the stimulation of the immune system, and in the treatment of cancers, such as those currently treated using native human IL-2 or Proleukin® IL-2. The human IL-2 muteins of the present invention and suitable biologically active variants thereof have the advantage of reducing pro-inflammatory cytokine production predictive of having lower toxicity, while maintaining or enhancing desirable functional activities such as NK cell proliferation, survival, NK-mediated cytotoxicity (NK, LAK, and ADCC), and T cell proliferation.

Because of their predicted lower toxicity, in those clinical indications requiring high doses of IL-2, the human IL-2 muteins of the present invention, and biologically active variants thereof, can be administered at similar or higher doses than can native IL-2 or Proleukin® IL-2 while minimizing toxicity effects. Thus, the present invention provides a method for reducing interleukin-2 (IL-2)-induced toxicity symptoms in a subject undergoing IL-2 administration as a treatment protocol, where the method comprising administering the IL-2 as an IL-2 mutein disclosed herein. Furthermore, the human IL-2 muteins of the present invention and suitable biologically active variants thereof have the additional advantage of greater therapeutic efficacy, so that lower doses of these human IL-2 muteins can provide greater therapeutic efficacy than comparable doses of native IL-2 or Proleukin® IL-2.

A pharmaceutically effective amount of an IL-2 mutein pharmaceutical composition of the invention is administered to a subject. By “pharmaceutically effective amount” is intended an amount that is useful in the treatment, prevention or diagnosis of a disease or condition. By “subject” is intended mammals, e.g., primates, humans, dogs, cats, cattle, horses, pigs, sheep, and the like. Preferably the subject undergoing treatment with the pharmaceutical formulations of the invention is human.

When administration is for the purpose of treatment, administration may be for either a prophylactic or therapeutic purpose. When provided prophylactically, the substance is provided in advance of any symptom. The prophylactic administration of the substance serves to prevent or attenuate any subsequent symptom. When provided therapeutically, the substance is provided at (or shortly after) the onset of a symptom. The therapeutic administration of the substance serves to attenuate any actual symptom.

Thus, for example, formulations comprising an effective amount of a pharmaceutical composition of the invention comprising a human IL-2 mutein of the invention or biologically active variant thereof can be used for the purpose of treatment, prevention, and diagnosis of a number of clinical indications responsive to therapy with IL-2. The human IL-2 muteins of the present invention and biologically active variants thereof can be formulated and used in the same therapies as native-sequence IL-2 or Proleukin® IL-2. Accordingly, formulations of the invention comprising a human IL-2 mutein of the invention or biologically active variant thereof are useful for the diagnosis, prevention, and treatment (local or systemic) of bacterial, viral, parasitic, protozoan and fungal infections; for augmenting cell-mediated cytotoxicity; for stimulating lymphokine activated killer (LAK) cell activity; for mediating recovery of immune function of lymphocytes; for augmenting alloantigen responsiveness; for facilitating immune reconstitution in cancer patients following radiotherapy, or following or in conjunction with chemotherapy alone or in combination with other anti-cancer agents, or following or in conjunction with bone marrow or autologuos stem cell transplantation; for facilitating recovery of immune function in acquired immune deficient states; for reconstitution of normal immunofunction in aged humans and animals; in the development of diagnostic assays such as those employing enzyme amplification, radiolabelling, radioimaging, and other methods known in the art for monitoring IL-2 levels in the diseased state; for the promotion of T-cell growth in vitro for therapeutic and diagnostic purposes; for blocking receptor sites for lymphokines; and in various other therapeutic, diagnostic and research applications. The various therapeutic and diagnostic applications of human IL-2 or variants thereof, such as IL-2 muteins, have been investigated and reported in Rosenberg et al. (1987) N. Engl. J. Med. 316:889-897; Rosenberg (1988) Ann. Surg. 208:121-135; Topalian et al. 1988) J. Clin. Oncol. 6:839-853; Rosenberg et al. (1988) N. Engl. J. Med. 319:1676-1680; Weber et al. (1992) J. Clin. Oncol. 10:33-40; Grimm et al. (1982) Cell. Immunol. 70(2):248-259; Mazumder (1997) Cancer J. Sci. Am. 3(Suppl. 1):S37-42; Mazumder and Rosenberg (1984) J. Exp. Med. 159(2):495-507; and Mazumder et al. (1983) Cancer Immunol. Immunother. 15(1): 1-10. Formulations of the invention comprising a human IL-2 mutein of the invention or biologically active variant thereof may be used as the single therapeutically active agent or may be used in combination with other immunologically relevant cells or other therapeutic agents. Examples of relevant cells are B or T cells, NK cells, LAK cells, and the like, and exemplary therapeutic reagents that may be used in combination with IL-2 or variant thereof are the various interferons, especially gamma interferon, B-cell growth factor, IL-1, and antibodies, including, but not limited to, anti-HER2 antibodies such as Herceptin® (Genentech, Inc., San Francisco, Calif.) or anti-CD20 antibodies such as Rituxan® (Rituximab; IDEC-C2B8; IDEC Pharmaceuticals Corp., San Diego, Calif.).

The amount of human IL-2 mutein or biologically active variant thereof administered may range between about 0.1 to about 15 mIU/m². Therapeutically effective doses and particular treatment protocols for IL-2 immunotherapy in combination with anti-cancer monoclonal antibodies are known in the art. See, for example, the doses and treatment protocols disclosed in copending U.S. Patent Application Publication Nos. 2003-0185796, entitled Methods of Therapy for Non-Hodgkin's Lymphoma,” and 20030235556, entitled “Combination IL-2/Anti-HER2 Antibody Therapy for Cancers Characterized by Overexpression of the HER2 Receptor Protein, and copending U.S. Patent Application No. 60/491,371, entitled “Methods of Therapy for Chronic Lymphocytic Leukemia,” Attorney Docket No. 59516-278, filed Jul. 31, 2003; the contents of which are herein incorporated by reference in their entirety. For indications such as renal cell carcinoma and metastatic melanoma, the human IL-2 mutein or biologically active variant thereof may be administered as a high-dose intravenous bolus at 300,000 to 800,000 IU/kg/8 hours. See the foregoing U.S. patent applications for recommended doses for IL-2 immunotherapy for B-cell lymphomas, HER2+cancers such as breast cancer, and CLL.

Use of IL-2 immunotherapy for the treatment of HIV infection is also known in the art. See, for example, U.S. Pat. No. 6,579,521, herein incorporated by reference in its entirety, for recommended doses and protocols for this clinical indication.

Thus, the invention provides a method for the treatment of cancer in a subject or for modulating the immune response in a subject, comprising administering a therapeutically effective amount of a human IL-2 mutein of the invention or biologically active variant thereof. The “therapeutically effective amount” refers to a dosage level sufficient to induce a desired biological result without inducing unacceptable toxicity effects. Amounts for administration may vary based upon the concentration of human IL-2 mutein or variant thereof within the pharmaceutical composition, the desired activity, the disease state of the mammal being treated, the dosage form, method of administration, frequency of administration, and patient factors such as age, sex, and severity of disease. It is recognized that a therapeutically effective amount is provided in a broad range of concentrations, and that the subject may be administered as many therapeutically effective doses as is required to reduce and/or alleviate the signs, symptoms, or causes of the disorder in question, or bring about any other desired alteration of a biological system. Generally, an IL-2 mutein pharmaceutical composition of the invention will comprise the human IL-2 mutein or variant thereof in a concentration range which is greater than that used for Proleukin® IL-2. As the doses are increased relative to that of Proleukin® IL-2, the subject should be closely monitored to determine if toxic side effects appear. Such clinical experimental analyses are well-known to those of skill in the art, and would, for example, have been used to established the current doses of Proleukin® IL-2 for use in immunomodulation and cancer therapy.

Bioassays for Monitoring Functional Activity of Human IL-2 Muteins

The present invention also provides novel bioassays for monitoring IL-2 induced NK cell proliferation and TNF-α production, IL-2-induced NK cell-mediated cytotoxicity, IL-2-induced T cell proliferation, and IL-2-induced NK cell survival. These assays have been developed to screen candidate IL-2 muteins for the desired functional profile of reduced pro-inflammatory cytokine production (particularly TNF-α and/or IFN-γ) so as to improve tolerability, and improved NK cell-mediated function as reflected in the ability of the mutein to maintain or increase NK and/or T cell proliferation, to maintain or increase NK-mediated cytotoxicity (NK, LAK, and ADCC), and to maintain or increase NK cell survival.

The first of these assays is referred to herein as the “NK-92 bioassay,” which monitors IL-2 induction of TNF-α production and IL-2-induced NK cell proliferation. This bioassay utilizes the human NK-92 cell line (ATCC CRL-2407, CMCC ID #11925). The NK-92 cell line, originally described by Gong et al. (1994) Leukemia 8(4):652-658, displays phenotypic and functional characteristics of activated NK cells. Proliferation of NK-92 is IL-2 dependent; cells will die if cultured in the absence of IL-2 for 72 hours. The cell line also produces detectable levels of TNF-α within 48-72 hours following exposure to IL-2.

In accordance with the methods of the present invention, candidate IL-2 muteins can be screened for relative ability to induce TNF-α production and induce NK cell proliferation using this NK-92 bioassay. In this manner, NK-92 cells are cultured in complete medium (NK-92 medium) consisting of Alpha-MEM, 12% heat-inactivated fetal bovine serum (FBS), 8% heat-inactivated horse serum, 0.02 mM folic acid, 0.2 mM inositol, 2 mM L-glutamine, and 0.1 mM β-mercaptoethanol. Cultures are seeded at a minimum density of 1-3×10⁵ cells/ml and supplemented with 1000 IU/ml of the reference recombinant human IL-2 mutein (for example, the reference IL-2 mutein designated des-alanyl-1, C125S human IL-2 or the reference C125S human IL-2 mutein). In preparation for the assay, cells are placed in fresh NK-92 medium a minimum of 48 h prior to assay use. One day prior to assay, NK-92 are washed three times and placed in NK-92 medium without any supplemental IL-2 for 24 h. Cells are centrifuged, suspended in NK-92 medium (no IL-2) and plated into 96-well flat bottom plates at a density of 4×10⁴ cells/well in 200 μl with varying concentrations of the reference IL-2 mutein, for example, des-alanyl-1 C125S or C125S human IL-2, or varying concentrations of a candidate IL-2 mutein that is being screened for the functional profile of interest diluted in NK-92 medium. Following a 72-h incubation at 37° C., 5% CO₂, a 100 μl aliquot of culture supernatant is removed and frozen for subsequent quantification of TNF-α using a commercially available TNF-α ELISA kit (for example, BioSource Cytoscreen™ Human TNF-α ELISA kit; Camarillo, Calif.). For the remaining cells in culture, proliferation is determined using a commercially available MTT dye-reduction kit (CellTiter 96® Non-Radioactive Cell Proliferation Assay Kit (Promega Corp., Madison, Wis.), and a stimulation index is then calculated based on a colorimetric readout.

The second IL-2 bioassay disclosed herein provides a method for screening candidate IL-2 muteins for their ability to induce natural killer (NK) cell-mediated cytotoxicity. This bioassay, designated the “NK3.3 cytotoxicity bioassay,” utilizes the human NK3.3 cell line. The NK3.3 cell line displays phenotypic and functional characteristics of peripheral blood NK cells (Kornbluth (1982) J. Immunol. 129(6):2831-2837), and can mediate antibody-dependent cellular cytotoxicity (ADCC) via the Fc receptor (CD16, FcγRIIIA). Table 2 in the Experimental section below summarizes the biological activities of NK3.3 cells examined with this IL-2 bioassay.

In accordance with the methods of the present invention, candidate IL-2 muteins can be screened for their cytotoxicity activity using this NK3.3 cytotoxocity bioassay. In this manner, NK3.3 cells are expanded and maintained in RPMI-1640 medium supplemented with 15% heat-inactivated fetal bovine serum, 25 mM HEPES, 2 mM L-glutamine, and 20% Human T-Stim™ w/PHA as a source of IL-2. In preparation for the assay, NK3.3 cells are cultured in the absence of IL-2 (“starved) for 24 h. The assay consists of 5×10⁴ “starved” NK3.3 cells plated in U-bottom 96-well plates, exposed to varying concentrations of a reference IL-2 mutein, for example, des-alanyl-1, C125S or C125S human IL-2 mutein, or varying concentrations of a candidate IL-2 mutein of interest in a total volume of 200 μl. Following an 18-h incubation, the IL-2-stimulated NK3.3 effector cells are co-incubated with 5×10³ calcein AM-labeled target cells (K562 or Daudi) or antibody-coated, calcein AM-labeled targets (Daudi coated with rituximab at a final concentration of 2 μg/ml) to achieve a final effector-to-target ratio of 10:1. Following co-incubation of effector and target cells for 4 h, the 96 well plates are briefly centrifuged; 100 μl of culture supernatant is removed and placed into a black, clear, flat-bottom 96-well plate for quantitation of calcein AM release by fluorimeter. Quantitation is expressed as percent specific lysis, and is calculated by the following equation: % specific lysis=100×[(mean experimental−mean spontaneous release)/(mean maximal release−mean spontaneous release)]; whereby the spontaneous release is determined from wells containing labeled targets and no effectors, and maximal release is determined from wells containing labeled targets and 1% Triton X-100.

The third IL-2 bioassay disclosed herein provides a method for screening candidate IL-2 muteins for their ability to induce T cell proliferation. In this manner, this IL-2 bioassay for T-cell proliferation utilizes the human T-cell line Kit225 (CMCC ID#11234), derived from a patient with T-cell chronic lymphocytic leukemia (Hori et al. (1987) Blood 70(4):1069-1072). Kit225 cells constitutively express the α, β, γ subunits of the IL-2 receptor complex. Proliferation of Kit225 is IL-2 dependent; cells will die if cultured in the absence of IL-2 for an extended period of time.

In accordance with the present invention, the assay consists of culturing Kit225 cells in the absence of IL-2 for 24 h, followed by plating a specified number of cells with varying concentrations of the reference IL-2 mutein, for example, des-alanyl-1 C125S or C125S human IL-2 mutein, or varying concentrations of a candidate IL-2 mutein of interest. Following a 48-h incubation, proliferation is determined using a standard, commercially available MTT dye reduction kit, and a stimulation index is calculated based on a colorimetric readout.

The fourth IL-2 bioassay of the present invention provides a method for screening candidate IL-2 muteins for their ability to promote NK cell survival. In this manner, candidate muteins are screened for their ability to induce NK cell survival signaling. Proleukin® IL-2 (i.e., the formulation comprising the des-alanyl-1 C125S human IL-2 mutein) induces the phosphorylation of AKT in NK3.3 cells previously starved for IL-2, which is considered a “survival signal.” In accordance with this bioassay, NK3.3 cells are expanded and maintained in RPMI-1640 medium supplemented with 15% heat-inactivated fetal bovine serum, 25 mM HEPES, 2 mM L-glutamine, and 20% Human T-Stim™ w/PHA as a source of IL-2. In preparation for assay, NK3.3 cells are cultured in the absence of IL-2 for 24 h. As an indicator of cell survival signaling, “starved” NK3.3 cells (2×10⁶) are stimulated by addition of 2 nM of the reference IL-2 mutein, for example, the des-alanyl-1 C125S or C125S human IL-2 mutein, or 2 nM of a candidate IL-2 mutein of interest, for 30 min. Cells are washed twice in phosphate buffered saline (PBS). The cell pellet is lysed in 50 μl of a cell extraction buffer containing protease inhibitors and subjected to one freeze-thaw cycle. The extract is centrifuged at 13,000 rpm for 10 min @ 4° C. An aliquot of the cleared lysate is added at a 1:10 dilution to wells of the AKT [pS473]* Immunoassay Kit (BioSource International). Following the manufacturer's protocol, levels of phosphorylated AKT are detected by quantitative ELISA.

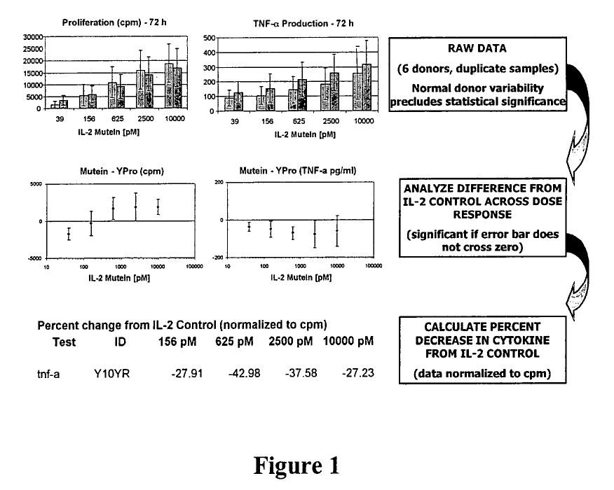

The present invention also provides bioassays for use in screening IL-2 muteins for their functional profiles using human peripheral blood mononuclear cells (PBMC). The first of these bioassays is a combination proliferation/pro-inflammatory cytokine production bioassay. Upon exposure to IL-2, human PBMC proliferate and secrete cytokines in a dose-dependent manner. This combination assay was designed to assess levels of proliferation and cytokine production following 72 h stimulation with a reference IL-2 mutein (such as the des-alanyl-1, C125S mutein or C125S mutein) or a candidate IL-2 mutein of interest. PBMC are isolated by density gradient separation (for example, using ACDA Vacutainer CPT tubes) from one or more normal human donors. In 96-well tissue-culture treated plates, 200,000 cells per well are incubated with various concentrations of IL-2 (0.039 nM-10 nM) or no IL-2 as a negative control in complete RPMI medium (RPMI, 10% heat-inactivated human AB serum, 25 mM HEPES, 2 mM glutamine, penicillin/streptomycin/fungizone) at 37° C., 7% CO₂. Following 66 h of incubation, an aliquot of cell culture supernatant is removed and frozen for cytokine detection at a later time. The cells are pulsed with 1 μCi ³H-thymidine for 6 h, and then harvested to determine levels of nucleotide incorporation (for example, using a Wallac Trilux Microbeta Plate Reader) as a measure of cell proliferation. Commercially available ELISA kits (for example, from BioSource International) can then be used to detect levels of TNF-α in the cell culture supernatants per manufacturer's guidelines. Repeating the assay for a complete panel of separate donors, for example, 6, 8, or 10 donors, provides a characterization of representative proliferative and cytokine responses to IL-2 in a “normal population.” Data can then be analyzed as shown in FIG. 1, and described further herein below in Example 10.

The second PBMC-based bioassay can be used to screen candidate IL-2 muteins for their ability to mediate effector cell cytotoxicity. In this assay, human PBMC are separated from whole blood using density gradient centrifugation. PBMC are stimulated for 3 days in the presence of 10 nM IL-2 control or IL-2 mutein of interest, to generate LAK activity as generally practiced in current state of the art (see for example Isolation of Human NK Cells and Generation of LAK activity IN: Current Protocols in Immunology; 1996 John Wiley & Sons, Inc). The resulting cell population contains “effector” cells, which may be classified as NK or LAK, and can kill K562 and Daudi tumor cell targets, respectively. These effector cells may also mediate ADCC, whereby the effector cells recognize the Fc portion of a specific antibody that is bound to the Daudi target cells. In one embodiment, the antibody bound to the Daudi target cells is Rituxan® (rituximab).

In accordance with the methods of the present invention, human PBMC (effector cells) that have been stimulated with a candidate IL-2 mutein of interest or a reference IL-2 control are co-incubated with calcein AM-labeled target cells at various effector to target cell (E:T ratios) for 4 h. The amount of cytotoxic activity is related to the detection of calcein AM in the culture supernatant. Quantitation is expressed as percent specific lysis at each E:T ratio, based upon determination of spontaneous and maximum release controls. This bioassay examines the following biological activities: natural/spontaneous cytotoxicity (NK), where the target is K562 cells; lymphokine-activated killing (LAK), where the target is Daudi cells; and antibody-dependent cellular cytotoxicity (ADCC), where the target is antibody-coated Daudi cells (for example, Rituxan®-coated Daudi cells).

Data is obtained from a fluorimeter and expressed in relative fluorescence untis (rfu). Controls for this bioassay include labeled target cells alone (min) and labeled target cells with final 1% Triton X-100 as a measure of 100% lysis (max). The percent min to max ratio is calculated using the following equation as a measure of assay validity (assay invalid if >30%):

$$\% min to max = 100 ⨯ \frac{mean spontaneous release rfu}{mean maximum release rfu}$$

Once the assay is deemed valid, the mean and standard deviation for triplicate sample points is calculated, followed by the percent specific lysis from mean of triplicate points using the following equation:

$$\% lysis = 100 ⨯ \frac{\begin{matrix}mean experimental rfu - \\ mean spontaneous release rfu\end{matrix}}{\begin{matrix}mean maximal release rfu - \\ mean spontaneous release rfu\end{matrix}}$$

Data is then reported as % specific lysis; in addition, the ratio of candidate IL-2 mutein to relevant IL-2 reference control (for example, des-alanyl-1, C125S human IL-2 mutein or C125S human IL-2 mutein) can be used to determine whether cytotoxic activity is maintained relative to the IL-2 reference control in a mixed population of human PBMC donors.

The foregoing assays can be utilized to screen candidate IL-2 mutein libraries for desired functional profiles, where the functional activities of interest include one or more of the following: IL-2 induced pro-inflammatory cytokine production (particularly TNF-α and/or IFN-γ), IL-2 induced NK and/or T cell proliferation, IL-2 induced NK-mediated cytotoxicity (NK, LAK, and ADCC), and IL-2 induced NK cell survival.

The following examples are offered by way of illustration and not by way of limitation.

### EXPERIMENTAL

The therapeutic utility of IL-2 is hampered by the toxicities associated with its administration, including fevers, chills, hypotension, and vascular leak syndrome. IL-2 muteins with improved tolerability and IL-2-mediated NK and T effector functions would allow for administration of similar therapeutic doses that are better tolerated of higher therapeutic doses, thereby increasing the potential for greater therapeutic efficacy of this protein. The overall strategy of the work presented herein was to select novel human IL-2 muteins that exhibit the following functional profile using a comprehensive panel of specialized moderate throughput human NK cell-based immunoassay screening systems: reduced pro-inflammatory cytokine production (particularly TNF-α and/or IFN-γ) so as to improve tolerability, and improved NK cell-mediated function as reflected in the ability of the mutein to maintain or increase NK and/or T cell proliferation, to maintain or increase NK-mediated cytotoxicity (NK, LAK, and ADCC), and to maintain or increase NK cell survival.

For purposes of identifying suitable IL-2 muteins with the desired therapeutic profile, the biological activities of the candidate recombinant human IL-2 muteins were compared to these biological activities exhibited by des-alanyl-1, C125S human IL-2 (abbreviated as “Pro” in the examples below) and C125S human IL-2 (abbreviated as “Ala-Pro” in the examples below), which are referred to as the reference IL-2 muteins. The recombinantly E. coli-produced des-alanyl-1, C125S human IL-2 mutein, which is aldesleukin, is marketed as a formulation under the tradename Proleukin® IL-2 (Chiron Corporation, Emeryville, Calif.). Proleukin® IL-2 is a specific lyophilized formulation that uses an unglycosylated form of the mutein that has been produced in E. coli, and was reconstituted in distilled water for use in the bioassays described herein below. In certain experiments, a monomeric formulation of aldesleukin marketed under the tradename L2-7001® IL-2 (Chiron) is used, which is a liquid formulation comprising the same human IL-2 mutein (aldesleukin) as Proleukin® IL-2, but differing in the final purification steps prior to its formulation. See U.S. Pat. No. 4,931,543 and U.S. Pat. No. 6,525,102. The C125S human IL-2 used in the initial screening experiments was produced in the AME mammalian system, and was formulated in proprietary AME buffer.

The human IL-muteins described herein below were expressed in host mammalian 293T cells. Where the reference IL-2 mutein was C125S human IL-2, the host cells had been transformed with an expression construct comprising the native human IL-2 coding sequence with a C125S mutation operably linked to the Pro-1 promoter. The coding sequence comprised the authentic IL-2 signal sequence and codon for the N-terminal alanine of human IL-2 (i.e., nucleotides 1-63 of SEQ ID NO:1) fused at the coding sequence for des-alanyl-1, C125S human IL-2 (i.e., SEQ ID NO:7). The protein was expressed as GSHis-tagged protein in the 293T cell mammalian expression system and purified with NI-NTA beads.

### Example 1

### Initial Screening of Human IL-2 Muteins

A library comprising all 2,508 possible single amino acid mutein variants of the C125S human IL-2 molecule (designated “Ala-Pro” in the examples herein) was constructed using a codon-based mutagenesis technology platform (Applied Molecular Evolution, Inc., San Diego, Calif.). Ala-Pro differs from the des-alanyl-1 C125S human IL-2 mutein utilized in the commercially available Proleukin® IL-2 product in having the N-terminal Ala residue at position 1 of the native human IL-2 sequence retained in the C125S human IL-2 mutein. The AME mammalian expression systems DirectAME™ and ExpressAME™ (Applied Molecular Evolution, Inc., San Diego, Calif.) were utilized in the recombinant production of the Ala-Pro muteins.

The primary screen was carried out using a human NK-92 cell line-based functional immunoassay, in which pro-inflammatory cytokine production (TNF-a), NK cell proliferation, and NK cytolytic killing (NK, LAK, and ADCC), and cell survival (pAKT; NK3.3 cell line) were assayed. The primary functional endpoints selected included: (1) reduced pro-inflammatory TNF-α production by the human NK-92 cell line relative to that observed with Ala-Pro IL-2 (i.e., C125S human IL-2 mutein) or Proleukin®IL-2 (i.e., des-alanyl-1, C125S human IL-2 mutein); (2) maintained or improved human NK-92 cell line proliferation relative to that observed with either of these two reference IL-2 muteins; and 3) maintained or improved human NK3.3 cell line-mediated NK-, LAK-, and ADCC-mediated cytolytic killing relative to that observed with either of these two reference IL-2 muteins. Secondary functional endpoints were maintained or improved induction of phosphorylated AKT (pAKT) in the NK3.3 cell line relative to that observed with either of these two reference IL-2 muteins, and maintained or improved T cell proliferation by the human Kit225 T cell line relative to that observed with Ala-Pro IL-2 (i.e., C25S human IL-2 mutein) or Proleukin® (i.e., des-alanyl-1, C125S human IL-2 mutein).

Out of all 2,508 possible single amino acid mutein variants of the human C125S IL-2 molecule, 168 were identified for further testing (see Table 1 above). Three classes of highly desirable IL-2 muteins with improved functional profiles were identified using this approach. All IL-2 muteins selected maintain NK cytolytic function (NK/LAK/ADCC) when compared to the des-alanyl-1, C125S (i.e., present in Proleukin® IL-2) or C125S (i.e., Ala-Pro) human IL-2 muteins.

The first class of muteins is predicted to have improved tolerability as evidenced by decreased induction of TNF-α production by NK cells relative to that observed with the des-alanyl-1 C125S human IL-2 mutein or C125S human IL-2 mutein. The muteins within this class fall within two categories: (1) those that induce low TNF-α production and maintain NK cell proliferation at concentrations of 50 pM to 1000 pM, which include the des-alanyl-1, C125S or C125S human IL-2 muteins further comprising the L72N substitution; and (2) those that induce low TNF-α production and maintain proliferation at high concentration (1 nM) only, which include the des-alanyl-1 C125S or C125S human IL-2 muteins further comprising the V91D or F42E substitution. See Example 8 and Tables 13 and 14, below.

The second class of muteins includes those that were identified as having increased NK cell function, particularly NK cell proliferation, relative to that observed with des-alanyl-1, C125S human IL-2 mutein (i.e., in Proleukin® IL-2) or the C125S human IL-2 mutein (designated Ala-Pro IL-2 herein). Muteins identified within this functional class include the des-alanyl-1, C125S or C125S human IL-2 muteins further comprising the L36D and L40D substitution. See Example 8 and Table 15, below.

The third class of muteins includes “bifunctional” muteins that are predicted to have improved tolerability based on the decreased induction of TNF-α while also increasing NK proliferation relative to that observed with the des-alanyl-1, C125S human IL-2 mutein present in Proleukin® IL-2 or the C125S human IL-2 mutein (designated Ala-Pro IL-2 herein). These “bifunctional” muteins exhibit an improved ratio of NK proliferation:TNF-α production of greater than 1.5. Muteins identified within this functional class include the des-alanyl-1, C125S or C125S human IL-2 muteins further comprising the L19D, F42R, or E61R substitution. See Example 8 and Table 16, below.

The screening process that led to the identification of the leading candidates fitting into these three functional classes is further described in the examples below. The following protocols were used in the screening process.

NK Cell Proliferation/TNF-α Production

The IL-2 bioassay for natural killer (NK) cell proliferation and TNF-α production utilizes the human NK-92 cell line (ATCC CRL-2407, CMCC ID #11925). The NK-92 cell line, originally described by Gong et al. (1994) Leukemia 8(4):652-658, displays phenotypic and functional characteristics of activated NK cells. Proliferation of NK-92 is IL-2 dependent; cells will die if cultured in the absence of IL-2 for 72 hours. The cell line also produces detectable levels of TNF-α within 48-72 hours following exposure to IL-2.

NK-92 cells were cultured in complete medium (NK-92 medium) consisting of Alpha-MEM, 12% heat-inactivated fetal bovine serum (FBS), 8% heat-inactivated horse serum, 0.02 mM folic acid, 0.2 mM inositol, 2 mM L-glutamine, and 0.1 mM β-mercaptoethanol. Cultures were seeded at a minimum density of 1-3×10⁵ cells/ml and supplemented with 1000 IU/ml recombinant human IL-2 mutein (des-alanyl-1, C125S human IL-2 (i.e., aldesleukin or Proleukin® IL-2; Chiron Corporation, Emeryville, Calif.) or C125S human IL-2 (recombinantly produced in the AME's mammalian expression system noted above). In preparation for the assay, cells were placed in fresh NK-92 medium a minimum of 48 h prior to assay use. One day prior to assay, NK-92 were washed three times and placed in NK-92 medium without any supplemental IL-2 for 24 h. Cells were centrifuged, suspended in NK-92 medium (no IL-2) and plated into 96-well flat bottom plates at a density of 4×10⁴ cells/well in 200 μl with varying concentrations of des-alanyl-1 C125S or C125S human IL-2 as the reference IL-2 molecule or varying concentrations of an IL-2 mutein of the invention diluted in NK-92 medium. Following a 72-h incubation at 37° C., 5% CO₂, a 100 μl aliquot of culture supernatant was removed and frozen for subsequent quantification of TNF-α using a commercially available TNF-α ELISA kit (BioSource Cytoscreen™ Human TNF-α ELISA kit; Camarillo, Calif.). For the remaining cells in culture, proliferation was determined using a commercially available MTT dye-reduction kit (CellTiter 96® Non-Radioactive Cell Proliferation Assay Kit (Promega Corp., Madison, Wis.), and a stimulation index was then calculated based on a colorimetric readout.

NK Cell-Mediated Cytotoxicity

The IL-2 bioassay for natural killer (NK) cell-mediated cytotoxicity utilizes the human NK3.3 cell line. The NK3.3 cell line displays phenotypic and functional characteristics of peripheral blood NK cells (Kornbluth (1982) J. Immunol. 129(6):2831-2837), and can mediate antibody-dependent cellular cytotoxicity (ADCC) via the Fc receptor (CD16, FcγRIIIA). The cell line was obtained from Jackie Kornbluth, Ph.D., under limited use license agreement with St. Louis University, and deposited to CMCC (ID 12022).

Table 2 summarizes the biological activities of NK3.3 cells examined with this IL-2 bioassay.

TABLE 2
Biological activities of NK3.3 cells examined with IL-2 bioassay.

| ACTIVITY | EFFECTOR | TARGET | DESCRIPTION |
|---|---|---|---|
| NK | NK3.3 | K562 | Natural cytotoxicity |
| LAK | NK3.3 | Daudi | IL-2 activated killing |
| ADCC | NK3.3 | Daudi + Rituxan ® | Antibody-dependent |
| | | | cellular cytotoxicity |

NK3.3 cells were expanded and maintained in RPMI-1640 medium supplemented with 15% heat-inactivated fetal bovine serum, 25 mM HEPES, 2 mM L-glutamine, and 20% Human T-Stim™ w/PHA as a source of IL-2. In preparation for the assay, NK3.3 cells were cultured in the absence of IL-2 (“starved) for 24 h. The assay consists of 5×10⁴ “starved” NK3.3 cells plated in U-bottom 96-well plates, exposed to varying concentrations of des-alanyl-1, C125S or C125S human IL-2 as the reference IL-2 molecule or varying concentrations of an IL-2 mutein of the invention in a total volume of 200 μl. Following an 18-h incubation, the IL-2-stimulated NK3.3 effector cells were co-incubated with 5×10³ calcein AM-labeled target cells (K562 or Daudi) or antibody-coated, calcein AM-labeled targets (Daudi coated with rituximab at a final concentration of 2 μg/ml) to achieve a final effector-to-target ratio of 10:1. Following co-incubation of effector and target cells for 4 h, the 96 well plates were briefly centrifuged; 100 μl of culture supernatant was removed and placed into a black, clear, flat-bottom 96-well plate for quantitation of calcein AM release by fluorimeter. Quantitation was expressed as percent specific lysis, and was calculated by the following equation: % specific lysis=100×[(mean experimental−mean spontaneous release)/(mean maximal release−mean spontaneous release)]; whereby the spontaneous release was determined from wells containing labeled targets and no effectors, and maximal release was determined from wells containing labeled targets and 1% Triton X-100.

T-Cell Proliferation

The IL-2 bioassay for T-cell proliferation utilizes the human T-cell line Kit225 (CMCC ID#11234), derived from a patient with T-cell chronic lymphocytic leukemia (Hori et al. (1987) Blood 70(4):1069-1072). Kit 225 cells constitutively express the α, β, γ subunits of the IL-2 receptor complex. Proliferation of Kit225 is IL-2 dependent; cells will die if cultured in the absence of IL-2 for an extended period of time. The assay consists of Kit225 cells, cultured in the absence of IL-2 for 24 h, followed by plating a specified number of cells with varying concentrations of des-alanyl-1 C125S or C125S human IL-2 as the reference IL-2 molecule or varying concentrations of an IL-2 mutein of the invention. Following a 48-h incubation, proliferation was determined using a standard, commercially available MTT dye reduction kit, and a stimulation index was calculated based on a colorimetric readout.

NK Cell Survival Signaling

A subset of the human IL-2 mutein library was screened for the ability to induce NK cell survival signaling. Proleukin® IL-2 (i.e., aldesleukin, the des-alanyl-1 C125S human IL-2 mutein) induces the phosphorylation of AKT in NK3.3 cells previously starved for IL-2, which is considered a “survival signal.” NK3.3 cells were expanded and maintained in RPMI-1640 medium supplemented with 15% heat-inactivated fetal bovine serum, 25 mM HEPES, 2 mM L-glutamine, and 20% Human T-Stim™ w/PHA as a source of IL-2. In preparation for assay, NK3.3 cells were cultured in the absence of IL-2 for 24 h. As an indicator of cell survival signaling, “starved” NK3.3 cells (2×10⁶) were stimulated by addition of 2 nM of des-alanyl-1 C125S or C125S human IL-2 as the reference IL-2 molecule or 2 nM of an IL-2 mutein of the invention, for 30 min. Cells were washed twice in phosphate buffered saline (PBS). The cell pellet was lysed in 50 μl of a cell extraction buffer containing protease inhibitors and subjected to one freeze-thaw cycle. The extract was centrifuged at 13,000 rpm for 10 min @ 4° C. An aliquot of the cleared lysate was added at a 1:10 dilution to wells of the AKT [pS473]* Immunoassay Kit (BioSource International). Following the manufacturer's protocol, levels of phosphorylated AKT were detected by quantitative ELISA.

### Example 2

### Identification of IL-2 Muteins Based on Enhanced NK Cell Proliferation

Out of the 168 muteins identified for further screening (Table 1), a total of 97 beneficial mutations that augment NK cell proliferation to greater than 130% of that exhibited by des-alanyl-1 C125S human IL-2 (i.e., mutein present in Proleukin IL-2) at 0.1 nM without concomitantly increasing TNF-α production (i.e., less than 100±8% of the TNF-alpha production mediated by the des-alanyl-1, C125S human IL-2 mutein at 1 nM). These muteins are listed in Table 3.

TABLE 3
IL-2 muteins identified using NK-92 cell proliferation assay (CPA) as the primary selection
criterion. Total TNF-α production (pg/ml) at 1.0 nM protein and TNF-α production as a percentage of that
observed for des-alanyl-1, C125S human IL-2 (% Pro) are shown. CPA values are expressed as a
percentage of that observed for des-alanyl-,1 C125S human IL-2 (% Pro). The ratio of % NK cell
proliferation at 0.1 nM protein relative to % TNF-α production at 1.0 nM protein is shown (% CPA
0.1:TNF-α). Cytotoxicity assay values are expressed as a ratio of the values observed for des-alanyl-1,
C125S human IL-2 (:Pro) or for C125S human IL-2 (:Ala-Pro).

| | | | | | | Cytotoxicity Assay | | | | | |
|---|---|---|---|---|---|---|---|---|---|---|---|
| | | | CPA | CPA | | | | | | ADCC | |
| | TNF-α | TNF-α | % Pro | % Pro | | NK (K562) | | LAK (Daudi) | | (Daudi + Ritux) | |
| Mutation | pg/ml | % Pro | 0.1 nM | 1 nM | % CPA 0.1:TNF-α | :Pro | :Ala-Pro | :Pro | :Ala-Pro | :Pro | :Ala-Pro |
| T7A | 78.2 | 95.3 | 150.9 | 115.6 | 1.58 | 0.75 | 0.81 | 0.75 | 0.85 | 0.89 | 0.95 |
| T7D | 81.2 | 99.1 | 154.4 | 113.7 | 1.56 | 0.81 | 0.87 | 0.83 | 0.95 | 0.91 | 0.98 |
| T7R | 78.7 | 96.0 | 152.8 | 110.1 | 1.59 | 0.77 | 0.83 | 0.77 | 0.89 | 0.83 | 0.89 |
| K8L | 76.7 | 93.5 | 153.8 | 112.3 | 1.65 | 0.75 | 0.81 | 0.79 | 0.90 | 0.84 | 0.90 |
| K9A | 79.7 | 97.3 | 159.6 | 115.1 | 1.64 | 0.79 | 0.86 | 0.82 | 0.93 | 0.89 | 0.95 |
| K9D | 77.8 | 94.6 | 159.4 | 114.5 | 1.69 | 0.88 | 0.95 | 0.87 | 0.99 | 0.89 | 0.95 |
| K9R | 78.1 | 95.4 | 151.6 | 113.2 | 1.59 | 0.77 | 0.83 | 0.77 | 0.88 | 0.86 | 0.92 |
| K9S | 74.1 | 90.4 | 169.0 | 113.6 | 1.87 | 0.92 | 0.99 | 0.81 | 0.92 | 0.84 | 0.90 |
| K9V | 79.7 | 97.4 | 162.1 | 113.0 | 1.67 | 1.01 | 1.09 | 0.85 | 0.97 | 0.84 | 0.90 |
| K9W | 77.9 | 94.9 | 156.2 | 115.0 | 1.65 | 0.78 | 0.93 | 0.89 | 0.95 | 0.85 | 0.92 |
| T10K | 77.9 | 94.6 | 167.6 | 123.3 | 1.77 | 0.85 | 0.91 | 0.77 | 0.88 | 0.76 | 0.81 |
| T10N | 77.9 | 94.9 | 163.8 | 119.0 | 1.73 | 0.82 | 0.98 | 0.90 | 0.95 | 0.87 | 0.94 |
| Q11A | 73.8 | 89.9 | 153.4 | 116.8 | 1.71 | 0.78 | 0.85 | 0.84 | 0.96 | 0.86 | 0.92 |
| Q11R | 73.4 | 89.5 | 150.6 | 112.7 | 1.68 | 1.01 | 1.09 | 0.94 | 1.08 | 0.88 | 0.94 |
| Q11T | 76.9 | 93.7 | 152.3 | 105.1 | 1.63 | 1.05 | 1.13 | 0.91 | 1.04 | 0.94 | 1.00 |
| H16E | 64.8 | 98.9 | 153.8 | 92.2 | 1.56 | 1.21 | 1.36 | 0.84 | 0.98 | 1.06 | 1.15 |
| H16D | 38.2 | 72.4 | 131.2 | 97.4 | 1.56 | 0.75 | 1.08 | 0.91 | 1.16 | 0.96 | 1.02 |
| L19D | 42.6 | 80.7 | 140.9 | 97.6 | 1.56 | 0.85 | 1.08 | 0.81 | 1.00 | 0.92 | 0.99 |
| D20E | 45.8 | 88.4 | 130.8 | 93.5 | 1.56 | 0.90 | 1.13 | 0.94 | 1.12 | 1.07 | 1.19 |
| I24L | 55.8 | 107.4 | 136.6 | 101.2 | 1.56 | 1.18 | 1.62 | 0.97 | 1.13 | 1.09 | 1.22 |
| K32A | 160.1 | 96.4 | 166.0 | 89.5 | 1.72 | 0.78 | 1.06 | 0.83 | 1.05 | 0.96 | 1.00 |
| K32W | 167.9 | 98.5 | 155.6 | 77.7 | 1.58 | 0.63 | 0.76 | 0.79 | 0.91 | 0.92 | 0.99 |
| P34E | 161.9 | 96.2 | 176.4 | 105.6 | 1.83 | 0.82 | 1.12 | 0.92 | 1.15 | 0.99 | 1.02 |
| P34R | 165.8 | 98.6 | 157.5 | 92.3 | 1.60 | 0.68 | 0.92 | 0.82 | 1.03 | 0.94 | 0.97 |
| P34S | 161.0 | 92.8 | 157.3 | 97.2 | 1.70 | 0.71 | 0.96 | 0.82 | 1.03 | 1.00 | 1.03 |
| P34T | 163.1 | 96.2 | 167.0 | 106.9 | 1.74 | 0.77 | 1.05 | 0.88 | 1.10 | 1.05 | 1.09 |
| P34V | 158.7 | 95.6 | 173.5 | 99.2 | 1.81 | 0.76 | 1.03 | 0.85 | 1.07 | 0.99 | 1.02 |
| K35D | 173.5 | 99.2 | 191.2 | 106.4 | 1.93 | 0.88 | 1.06 | 0.94 | 1.08 | 0.95 | 1.03 |
| K35I | 147.2 | 95.9 | 152.1 | 94.1 | 1.59 | 0.67 | 0.92 | 0.82 | 1.04 | 0.99 | 1.02 |
| K35L | 162.3 | 96.2 | 161.1 | 101.3 | 1.67 | 0.67 | 0.91 | 0.89 | 1.12 | 1.01 | 1.04 |
| K35M | 157.9 | 93.1 | 173.4 | 108.0 | 1.86 | 0.79 | 1.08 | 0.94 | 1.18 | 1.06 | 1.09 |
| K35N | 165.1 | 97.0 | 187.6 | 109.7 | 1.93 | 0.83 | 1.13 | 0.86 | 1.08 | 1.02 | 1.05 |
| K35P | 172.3 | 95.4 | 188.1 | 106.8 | 1.97 | 0.76 | 0.91 | 0.85 | 0.98 | 0.93 | 1.01 |
| K35Q | 182.0 | 100.3 | 179.9 | 109.9 | 1.79 | 0.76 | 0.91 | 0.86 | 0.99 | 0.97 | 1.05 |
| K35T | 179.2 | 99.8 | 170.5 | 112.9 | 1.71 | 0.65 | 0.84 | 0.79 | 0.97 | 0.97 | 1.03 |
| L36A | 157.1 | 94.7 | 181.3 | 97.7 | 1.91 | 0.65 | 0.89 | 0.80 | 1.01 | 0.82 | 0.85 |
| L36D | 150.2 | 88.2 | 208.8 | 96.5 | 2.37 | 1.03 | 1.40 | 0.83 | 1.04 | 0.95 | 0.98 |
| L36E | 150.4 | 86.5 | 216.0 | 108.1 | 2.50 | 0.94 | 1.28 | 0.84 | 1.06 | 1.01 | 1.04 |
| L36F | 153.2 | 90.2 | 188.3 | 104.9 | 2.09 | 0.84 | 1.14 | 0.77 | 0.97 | 0.94 | 0.97 |
| L36I | 163.9 | 91.9 | 181.9 | 111.8 | 1.98 | 0.81 | 0.97 | 0.89 | 1.02 | 0.97 | 1.05 |
| L36K | 167.5 | 91.9 | 193.2 | 114.3 | 2.10 | 0.85 | 1.02 | 0.88 | 1.02 | 0.93 | 1.01 |
| L36M | 157.9 | 89.9 | 193.9 | 113.7 | 2.16 | 0.72 | 0.93 | 0.81 | 1.00 | 0.94 | 0.99 |
| L36N | 157.1 | 90.2 | 201.4 | 110.1 | 2.23 | 0.79 | 1.02 | 0.83 | 1.03 | 0.96 | 1.01 |
| L36P | 40.1 | 76.8 | 132.7 | 113.8 | 1.73 | 1.24 | 1.52 | 1.04 | 1.26 | 0.95 | 1.03 |
| L36S | 41.7 | 80.3 | 131.7 | 115.2 | 1.64 | 0.66 | 0.91 | 0.69 | 0.81 | 0.94 | 1.05 |
| L36W | 160.7 | 93.0 | 185.9 | 95.0 | 2.00 | 0.89 | 1.07 | 0.90 | 1.03 | 0.98 | 1.06 |
| L36Y | 170.3 | 95.6 | 177.6 | 96.3 | 1.86 | 0.93 | 1.13 | 0.96 | 1.11 | 0.95 | 1.03 |
| R38G | 109.5 | 95.4 | 150.7 | 91.3 | 1.58 | 0.66 | 0.84 | 0.83 | 0.89 | 0.95 | 0.96 |
| R38N | 44.1 | 85.0 | 132.7 | 100.8 | 1.56 | 1.03 | 1.28 | 0.94 | 1.12 | 0.94 | 1.05 |
| R38P | 45.8 | 88.8 | 135.8 | 101.3 | 1.53 | 1.17 | 1.44 | 0.87 | 1.05 | 0.91 | 0.99 |
| R38S | 43.4 | 83.7 | 136.3 | 100.0 | 1.63 | 0.95 | 1.17 | 0.96 | 1.15 | 0.90 | 0.97 |
| L40D | 43.8 | 84.9 | 140.2 | 112.0 | 1.65 | 1.05 | 1.29 | 0.96 | 1.16 | 1.00 | 1.08 |
| L40G | 40.8 | 78.1 | 142.6 | 110.9 | 1.83 | 1.11 | 1.37 | 0.96 | 1.16 | 1.00 | 1.08 |
| L40N | 46.3 | 89.5 | 135.6 | 110.0 | 1.52 | 0.85 | 1.17 | 0.65 | 0.77 | 0.96 | 1.08 |
| L40S | 45.1 | 86.7 | 135.1 | 105.0 | 1.56 | 0.96 | 1.33 | 0.71 | 0.83 | 0.89 | 1.00 |
| T41E | 110.8 | 96.7 | 175.9 | 99.9 | 1.82 | 0.96 | 1.16 | 0.92 | 1.03 | 1.05 | 1.06 |
| T41G | 113.5 | 99.2 | 158.7 | 104.7 | 1.60 | 0.84 | 0.96 | 0.83 | 0.91 | 0.96 | 0.94 |
| F42A | 101.3 | 96.4 | 168.4 | 168.8 | 1.75 | 0.76 | 0.91 | 0.74 | 0.80 | 0.87 | 0.88 |
| K64D | 131.1 | 91.9 | 152.5 | 109.4 | 1.66 | 0.75 | 0.94 | 0.91 | 1.02 | 0.98 | 1.13 |
| K64E | 134.5 | 94.4 | 154.9 | 109.5 | 1.64 | 0.53 | 0.66 | 0.76 | 0.85 | 0.88 | 0.92 |
| K64Q | 135.2 | 95.0 | 150.7 | 107.4 | 1.59 | 0.69 | 0.86 | 0.81 | 0.90 | 1.17 | 1.34 |
| K64R | 135.0 | 94.8 | 152.0 | 106.3 | 1.60 | 0.71 | 0.90 | 0.96 | 1.08 | 0.96 | 1.10 |
| P65D | 134.8 | 94.4 | 174.4 | 117.3 | 1.85 | 0.65 | 0.79 | 0.82 | 0.97 | 0.91 | 0.94 |
| P65H | 123.1 | 87.0 | 210.2 | 105.1 | 2.42 | 0.61 | 0.77 | 0.89 | 1.00 | 1.21 | 1.39 |
| P65I | 132.4 | 93.7 | 204.5 | 101.8 | 2.18 | 0.61 | 0.76 | 0.90 | 1.01 | 1.00 | 1.14 |
| P65K | 84.4 | 59.8 | 149.8 | 103.9 | 2.51 | 0.46 | 0.58 | 0.78 | 0.87 | 0.98 | 1.12 |
| P65L | 102.9 | 72.4 | 175.7 | 104.2 | 2.43 | 0.38 | 0.47 | 0.63 | 0.71 | 0.83 | 0.87 |
| P65Q | 111.4 | 78.9 | 189.9 | 93.2 | 2.41 | 0.69 | 0.87 | 0.86 | 0.97 | 1.07 | 1.11 |
| P65R | 135.3 | 95.4 | 178.4 | 103.9 | 1.87 | 0.83 | 1.01 | 0.88 | 1.04 | 0.91 | 0.94 |
| P65S | 127.7 | 89.3 | 205.3 | 119.7 | 2.30 | 0.80 | 0.97 | 0.82 | 0.97 | 0.96 | 1.00 |
| P65W | 134.5 | 95.4 | 181.6 | 91.4 | 1.90 | 0.51 | 0.64 | 0.71 | 0.79 | 1.22 | 1.40 |
| P65Y | 129.6 | 91.9 | 194.9 | 99.9 | 2.12 | 0.60 | 0.75 | 0.81 | 0.91 | 1.28 | 1.47 |
| L66A | 137.0 | 97.0 | 141.8 | 103.9 | 1.46 | 0.87 | 1.09 | 0.97 | 1.08 | 1.24 | 1.43 |
| L66F | 135.1 | 95.4 | 157.9 | 105.8 | 1.66 | 0.70 | 0.88 | 0.84 | 0.94 | 0.94 | 0.98 |
| E67A | 128.5 | 91.1 | 168.6 | 98.2 | 1.85 | 0.68 | 0.83 | 0.75 | 0.89 | 0.82 | 0.84 |
| L72N | 43.2 | 75.7 | 134.1 | 109.0 | 1.77 | 0.89 | 1.19 | 0.85 | 1.09 | 0.92 | 1.00 |
| L72T | 50.7 | 88.8 | 137.0 | 107.9 | 1.54 | 0.76 | 1.02 | 0.90 | 1.15 | 0.98 | 1.07 |
| L80F | 54.3 | 95.2 | 130.8 | 107.2 | 1.37 | 0.85 | 1.15 | 0.92 | 1.10 | 0.99 | 1.06 |
| L80G | 54.9 | 96.1 | 139.3 | 107.4 | 1.45 | 0.80 | 1.02 | 0.94 | 1.11 | 0.92 | 1.02 |
| L80K | 52.1 | 91.3 | 149.7 | 109.7 | 1.64 | 0.99 | 1.26 | 0.88 | 1.04 | 0.96 | 1.07 |
| L80R | 56.0 | 98.1 | 135.4 | 101.6 | 1.38 | 1.21 | 1.63 | 1.06 | 1.36 | 1.02 | 1.11 |
| L80Y | 52.9 | 100.5 | 130.7 | 111.4 | 1.30 | 0.78 | 1.12 | 0.76 | 0.98 | 0.98 | 1.03 |
| V91A | 47.5 | 89.7 | 136.1 | 119.7 | 1.52 | 0.83 | 1.19 | 0.90 | 1.16 | 1.02 | 1.08 |
| V91E | 40.6 | 77.0 | 135.6 | 96.7 | 1.76 | 0.84 | 1.06 | 0.84 | 1.03 | 0.93 | 1.01 |
| V91F | 41.5 | 78.9 | 134.8 | 101.9 | 1.71 | 1.00 | 1.27 | 0.92 | 1.14 | 1.01 | 1.09 |
| V91G | 36.3 | 68.5 | 133.7 | 104.4 | 1.95 | 0.83 | 1.06 | 0.94 | 1.15 | 0.93 | 1.00 |
| V91Q | 49.0 | 93.4 | 130.3 | 101.6 | 1.40 | 0.77 | 1.07 | 1.04 | 1.22 | 0.93 | 1.07 |
| L94T | 43.1 | 81.3 | 133.0 | 117.7 | 1.64 | 0.95 | 1.20 | 0.96 | 1.18 | 1.02 | 1.10 |
| L94Y | 37.5 | 71.1 | 137.4 | 128.1 | 1.93 | 0.62 | 0.87 | 0.80 | 0.94 | 0.88 | 1.01 |
| E95D | 38.2 | 72.5 | 135.3 | 125.1 | 1.87 | 0.70 | 0.98 | 0.87 | 1.02 | 0.89 | 1.02 |
| E95G | 41.5 | 78.4 | 137.7 | 113.0 | 1.76 | 0.82 | 1.15 | 0.90 | 1.06 | 0.91 | 1.05 |
| N119Q | 10.5 | 27.4 | 323.6 | 618.3 | 11.81 | 0.81 | 1.04 | 0.58 | 0.65 | 0.78 | 0.83 |
| Y107H | 37.8 | 71.3 | 144.3 | 104.2 | 2.02 | 0.79 | 1.13 | 0.89 | 1.15 | 0.95 | 1.00 |
| Y107K | 33.9 | 64.1 | 131.5 | 112.0 | 2.05 | 0.78 | 0.99 | 0.80 | 0.98 | 0.93 | 1.00 |
| Y107R | 31.0 | 58.8 | 138.5 | 121.6 | 2.36 | 0.67 | 0.94 | 0.88 | 1.04 | 0.83 | 0.95 |
| T123S | 50.0 | 94.1 | 133.0 | 120.9 | 1.41 | 0.83 | 1.19 | 0.86 | 1.10 | 1.00 | 1.06 |
| T123C | 50.6 | 95.2 | 142.5 | 106.7 | 1.50 | 0.95 | 1.21 | 0.98 | 1.21 | 10.1 | 1.09 |

A secondary analysis was performed for IL-2 mutein preparations quantitated at <0.066 ng/μl. This analysis identified 4 additional mutations, all occurring at key positions 36, 40, and 65, in which the mutein induced NK cell proliferation greater than 150% of that mediated by the des-alanyl-1, C125S human IL-2 mutein (i.e., present in Proleukin® IL-2) at 0.1 nM, and induced TNF-α production at 1 nM that was ≦100% of that mediated by a similar amount of the des-alanyl-1 C125S human IL-2 mutein (i.e., 1 nM). This secondary analysis also identified 7 additional mutations, all occurring at key positions 36, 64, and 65, as eliciting slight increases in TNF-αproduction at 1 nM (about 101-109% of that observed with a similar amount of the des-alanyl-1, C125S human IL-2 reference molecule) while still inducing NK cell proliferation greater than 150% of that mediated by the des-alanyl-1, C125S human IL-2 mutein at 0.1 nM. TNF-alpha production. See Table 4.

TABLE 4
Additional IL-2 muteins identified using NK-92 cell proliferation assay (CPA) as the primary
selection criterion. Total TNF-α production (pg/ml) at 1.0 nM and TNF-α production as a percentage of
that observed for des-alanyl-1, C125S human IL-2 (% Pro) are shown. CPA values are expressed as a
percentage of that observed for des-alanyl-1, C125S human IL-2 (% Pro). The ratio of % NK cell
proliferation at 0.1 nM protein relative to % TNF-α production at 1.0 nM protein is shown (% CPA
0.1:TNF-α). Cytotoxicity assay values are expressed as a ratio of the values observed for des-alanyl-1,
C125S human IL-2 (:Pro) or for C125S human IL-2 (:Ala-Pro).

| | | | | | | Cytotoxicity Assay | | | | | |
|---|---|---|---|---|---|---|---|---|---|---|---|
| | | | CPA | CPA | | | | | | ADCC | |
| | TNF-α | TNF-α | % Pro | % Pro | | NK (K562) | | LAK (Daudi) | | (Daudi + Ritux) | |
| Mutation | pg/ml | % Pro | 0.1 nM | 1 nM | % CPA 0.1:TNF-α | :Pro | :Ala-Pro | :Pro | :Ala-Pro | :Pro | :Ala-Pro |
| L36G | 143.0 | 83.5 | 191.8 | 114.0 | 2.30 | 0.62 | 0.74 | 0.79 | 0.91 | 0.90 | 0.98 |
| L36H | 152.8 | 87.6 | 168.4 | 111.3 | 1.92 | 0.70 | 0.85 | 0.57 | 0.66 | 0.96 | 1.04 |
| L40G | 106.7 | 92.9 | 162.2 | 91.4 | 1.75 | 0.90 | 1.15 | 0.94 | 1.01 | 1.05 | 1.06 |
| P65F | 129.5 | 91.4 | 196.0 | 112.4 | 2.14 | 0.70 | 0.86 | 0.81 | 0.96 | 0.92 | 0.95 |
| L36R | 166.6 | 102.0 | 174.6 | 95.7 | 1.71 | 0.83 | 1.14 | 0.88 | 1.11 | 0.99 | 1.03 |
| K64G | 144.1 | 101.0 | 174.1 | 96.8 | 1.72 | 0.80 | 0.97 | 0.88 | 1.04 | 1.00 | 1.03 |
| K64L | 143.7 | 101.0 | 177.5 | 113.5 | 1.76 | 0.88 | 1.07 | 0.82 | 0.97 | 0.91 | 0.94 |
| P65E | 148.1 | 104.3 | 221.0 | 110.3 | 2.12 | 0.87 | 1.07 | 0.83 | 0.98 | 0.98 | 1.01 |
| P65G | 153.5 | 108.8 | 171.0 | 99.8 | 1.57 | 0.96 | 1.20 | 0.87 | 0.98 | 0.98 | 1.01 |
| P65T | 145.0 | 102.3 | 183.5 | 115.5 | 1.79 | 1.15 | 1.41 | 0.93 | 1.10 | 1.03 | 1.06 |
| P65V | 145.0 | 102.8 | 182.0 | 104.0 | 1.77 | 0.79 | 0.97 | 0.92 | 1.10 | 0.97 | 1.00 |

### Example 3

### Identification of IL-2 Muteins Based on Reduced TNF-α Production

Muteins were selected that elicited less than 87% of the TNF-α production of des-alanyl-1, C125S (i.e., mutein present in Proleukin® IL-2) or C125S human IL-2 mutein IL-2 (designated Ala-Pro IL-2), each at 1 nM, and that maintained (at least 96.4%) or enhanced NK cell proliferation as compared to des-alanyl-1, C125S human IL-2 at both 0.1 nM and 1 nM, and that maintained (at least 79.2%) NK cell proliferation relative to the C125S human IL-2 mutein at 0.1 nM (data not shown). See Table 5.

TABLE 5
IL-2 muteins identified using the following selection criteria: TNF-α production <87% of that
observed for des-alanyl-1, C125S human IL-2 (Pro) at 1.0 nM and NK cell proliferation at two
concentrations (0.1 and 1.0 nM) maintained or improved relative to that observed for des-alanyl-1, C125S
human IL-2 (Pro). Total TNF-α production (pg/ml) at 1.0 nM and TNF-α production as a percentage of
that observed for des-alanyl-1, C125S human IL-2 (% Pro) or C125S human IL-2 (% Ala-Pro) are shown.
CPA values are expressed as a percentage of that observed for des-alanyl-1, C125S human IL-2 (% Pro).
The ratio of % NK cell proliferation at 0.1 nM protein relative to % TNF-α production at 1.0 nM protein is
shown (% CPA 0.1:TNF-α). Cytotoxicity assay values are expressed as a ratio of the values observed for
des-alanyl-1, C125S human IL-2 (:Pro) or for C125S human IL-2 (:Ala-Pro).

| | | | | | | | Cytotoxicity Assay | | | | | |
|---|---|---|---|---|---|---|---|---|---|---|---|---|
| | | | TNF-α | CPA | CPA | | | | | | ADCC | |
| | TNF-α | TNF-α | % Ala- | % Pro | % Pro | % CPA | NK (K562) | | LAK (Daudi) | | (Daudi + Ritux) | |
| Mutation | pg/ml | % Pro | Pro | 0.1 nM | 1 nM | 0.1::TNF-α | :Pro | :Ala-Pro | :Pro | :Ala-Pro | :Pro | :Ala-Pro |
| H16D | 38.2 | 72.4 | 68.0 | 131.2 | 97.4 | 1.81 | 0.75 | 1.08 | 0.91 | 1.16 | 0.96 | 1.02 |
| L19D | 42.6 | 80.7 | 75.8 | 140.9 | 97.6 | 1.75 | 0.85 | 1.08 | 0.81 | 1.00 | 0.92 | 0.99 |
| L36A | 37.9 | 72.8 | 70.5 | 123.9 | 115.5 | 1.70 | 1.23 | 1.51 | 0.98 | 1.18 | 0.95 | 1.03 |
| L36D | 38.6 | 74.1 | 71.7 | 128.8 | 108.4 | 1.74 | 1.16 | 1.43 | 0.95 | 1.14 | 1.02 | 1.10 |
| L36G | 38.2 | 73.6 | 71.3 | 115.9 | 97.9 | 1.57 | 0.78 | 1.08 | 0.90 | 1.05 | 1.00 | 1.12 |
| L36N | 41.1 | 78.9 | 76.4 | 122.8 | 115.2 | 1.56 | 0.87 | 1.07 | 0.85 | 1.02 | 0.94 | 1.02 |
| L36P | 40.1 | 76.8 | 74.4 | 132.7 | 113.8 | 1.73 | 1.24 | 1.52 | 1.04 | 1.26 | 0.95 | 1.03 |
| R38D | 92.3 | 80.5 | 77.6 | 132.5 | 85.9 | 1.65 | 0.55 | 0.63 | 0.78 | 0.85 | 0.91 | 0.89 |
| L40G | 40.8 | 78.1 | 75.6 | 142.6 | 110.9 | 1.83 | 1.11 | 1.37 | 0.96 | 1.16 | 1.00 | 1.08 |
| F42E | 82.0 | 78.0 | 67.6 | 116.7 | 104.3 | 1.50 | 0.62 | 0.74 | 0.64 | 0.70 | 0.82 | 0.83 |
| F42R | 82.6 | 78.9 | 68.1 | 123.0 | 102.0 | 1.56 | 0.47 | 0.56 | 0.63 | 0.68 | 0.78 | 0.79 |
| F42A | 40.9 | 79.1 | 76.6 | 116.2 | 100.1 | 1.47 | 1.23 | 1.54 | 1.10 | 1.31 | 0.94 | 1.05 |
| F42T | 34.3 | 65.9 | 63.9 | 111.7 | 92.8 | 1.69 | 1.01 | 1.24 | 0.90 | 1.09 | 0.89 | 0.96 |
| F42V | 86.9 | 82.3 | 71.8 | 128.4 | 102.2 | 1.56 | 0.56 | 0.67 | 0.71 | 0.77 | 0.96 | 0.96 |
| K43H | 91.1 | 86.9 | 75.1 | 130.2 | 108.1 | 1.50 | 0.63 | 0.70 | 0.71 | 0.84 | 0.91 | 0.89 |
| F44K | 71.1 | 65.4 | 59.1 | 130.4 | 100.1 | 1.99 | 0.89 | 1.10 | 0.89 | 1.04 | 1.09 | 1.16 |
| M46I | 71.6 | 65.7 | 59.7 | 125.4 | 105.4 | 1.91 | 0.83 | 1.04 | 0.89 | 1.03 | 0.89 | 0.95 |
| E61K | 44.7 | 78.3 | 74.6 | 109.7 | 100.3 | 1.40 | 0.85 | 1.08 | 0.89 | 1.05 | 0.89 | 0.99 |
| E61R | 53.9 | 71.0 | 73.7 | 123.6 | 111.4 | 1.74 | 0.52 | 0.59 | 0.76 | 0.85 | 0.86 | 0.97 |
| P65K | 84.4 | 59.8 | 55/9 | 149.8 | 103.9 | 2.51 | 0.46 | 0.58 | 0.78 | 0.87 | 0.98 | 1.12 |
| P65L | 102.9 | 72.4 | 67.9 | 175.7 | 104.2 | 2.43 | 0.38 | 0.47 | 0.63 | 0.71 | 0.83 | 0.87 |
| P65N | 44.4 | 77.8 | 74.1 | 126.4 | 102.5 | 1.63 | 0.86 | 1.16 | 0.91 | 1.09 | 1.01 | 1.07 |
| P65Q | 111.4 | 78.9 | 73.8 | 189.9 | 93.2 | 2.41 | 0.69 | 0.87 | 0.86 | 0.97 | 1.07 | 1.11 |
| P65T | 42.8 | 75.1 | 71.5 | 127.6 | 108.2 | 1.70 | 0.87 | 1.11 | 0.89 | 1.05 | 1.02 | 1.13 |
| P65Y | 41.0 | 71.7 | 68.3 | 128.7 | 105.3 | 1.79 | 0.82 | 1.10 | 0.89 | 1.14 | 0.99 | 1.08 |
| E67A | 44.1 | 77.3 | 73.6 | 128.1 | 106.4 | 1.66 | 0.93 | 1.26 | 0.94 | 1.13 | 1.11 | 1.18 |
| L72G | 32.6 | 57.2 | 54.5 | 112.1 | 102.3 | 1.96 | 0.86 | 1.10 | 0.94 | 1.11 | 0.98 | 1.09 |
| L72N | 43.2 | 75.7 | 72.2 | 134.1 | 109.0 | 1.77 | 0.89 | 1.19 | 0.85 | 1.09 | 0.92 | 1.00 |
| L80V | 47.7 | 68.6 | 59.5 | 137.2 | 115.3 | 2.00 | 0.71 | 0.88 | 0.84 | 0.95 | 0.92 | 0.96 |
| R81K | 31.7 | 45.7 | 39.7 | 120.1 | 103.3 | 2.63 | 0.55 | 0.68 | 0.71 | 0.80 | 0.75 | 0.79 |
| N88D | 58.5 | 74.1 | 71.1 | 111.7 | 106.6 | 1.51 | 0.82 | 0.92 | 0.86 | 0.90 | 0.83 | 0.90 |
| V91D | 46.2 | 58.6 | 56.2 | 96.4 | 105.1 | 1.64 | 0.84 | 0.95 | 0.92 | 0.98 | 0.94 | 1.13 |
| V91G | 36.3 | 68.5 | 64.2 | 133.7 | 104.4 | 1.95 | 0.83 | 1.06 | 0.94 | 1.15 | 0.93 | 1.00 |
| V91E | 40.6 | 77.0 | 72.4 | 135.6 | 96.7 | 1.76 | 0.84 | 1.06 | 0.84 | 1.03 | 0.93 | 1.01 |
| V91F | 41.5 | 78.9 | 74.2 | 134.8 | 101.9 | 1.71 | 1.00 | 1.27 | 0.92 | 1.14 | 1.01 | 1.09 |
| V91W | 42.1 | 79.8 | 75.0 | 129.7 | 123.3 | 1.62 | 0.96 | 1.37 | 0.91 | 1.17 | 1.00 | 1.06 |
| L94I | 88.7 | 80.8 | 73.0 | 128.2 | 124.9 | 1.59 | 0.69 | 0.94 | 0.67 | 0.89 | 0.77 | 0.87 |
| L94Y | 37.5 | 71.1 | 66.8 | 137.4 | 128.1 | 1.93 | 0.62 | 0.87 | 0.80 | 0.94 | 0.88 | 1.01 |
| E95D | 38.2 | 72.5 | 68.1 | 135.3 | 125.1 | 1.87 | 0.70 | 0.98 | 0.87 | 1.02 | 0.89 | 1.02 |
| E95G | 41.5 | 78.4 | 73.6 | 137.7 | 113.0 | 1.76 | 0.82 | 1.15 | 0.90 | 1.06 | 0.91 | 1.05 |
| Y107H | 37.8 | 71.3 | 66.9 | 144.3 | 104.2 | 2.02 | 0.79 | 1.13 | 0.89 | 1.15 | 0.95 | 1.00 |
| Y107K | 33.9 | 64.1 | 60.2 | 131.5 | 112.0 | 2.05 | 0.78 | 0.99 | 0.80 | 0.98 | 0.93 | 1.00 |
| Y107R | 31.0 | 58.8 | 55.2 | 138.5 | 121.6 | 2.36 | 0.67 | 0.94 | 0.88 | 1.04 | 0.83 | 0.95 |
| N119Q | 10.5 | 27.4 | 27.0 | 323.6 | 618.3 | 11.82 | 0.81 | 1.04 | 0.58 | 0.65 | 0.78 | 0.83 |
| Q126V | 63.7 | 71.9 | 69.5 | 112.7 | 103.6 | 1.57 | 0.59 | 0.81 | 0.77 | 0.90 | 0.90 | 1.02 |

These screening criteria were adjusted to capture those muteins that met the criteria for TNF-α production less than 81% of that stimulated by des-alanyl-1, C125S or C125S human IL-2, each at 1 nM, and that maintained or enhanced NK cell proliferation (at least 95%) relative to des-alanyl-1, C125S human IL-2 at 1 nM (i.e., only at a single concentration of the reference IL-2 mutein). These screening criteria identified additional muteins that involved residue changes at positions 20, 78, 79, 80, 81, 88, and 126. See Table 6.

TABLE 6
IL-2 muteins identified using the following selection criteria: TNF-α production <81% of that
observed for des-alanyl-1, C125S human IL-2 (Pro), each at 1.0 nM, and NK cell proliferation at 1.0 nM
maintained or improved relative to des-alanyl-1, C125S human IL-2 mutein (Pro). Total TNF-α production
(pg/ml) at 1.0 nM and TNF-α production as a percentage of that observed for des-alanyl-1, C125S human
IL-2 (% Pro) or C125S human IL-2 (% Ala-Pro) are shown. CPA values are expressed as a percentage of
that observed for des-alanyl-1, C125S human IL-2 (% Pro). The ratio of % NK cell proliferation at 0.1 nM
protein relative to % TNF-α production at 1.0 nM protein is shown (% CPA 0.1:TNF-α). Cytotoxicity assay
values are expressed as a ratio of the values observed for des-alanyl-1, C125S human IL-2 (:Pro) or for
C125S human IL-2 (:Ala-Pro).

| | | | | | | | Cytotoxicity Assay | | | | | |
|---|---|---|---|---|---|---|---|---|---|---|---|---|
| | | | TNF-α | CPA | CPA | | | | | | ADCC | |
| | TNF-α | TNF-α | % Ala- | % Pro | % Pro | % CPA | NK (K562) | | LAK (Daudi) | | (Daudi + Ritux) | |
| Mutation | pg/ml | % Pro | Pro | 0.1 nM | 1 nM | 0.1:TNF-α | :Pro | :Ala-Pro | :Pro | :Ala-Pro | :Pro | :Ala-Pro |
| F78S | 51.3 | 74.0 | 64.2 | 52.4 | 114.0 | 0.71 | 0.46 | 0.57 | 0.66 | 0.74 | 0.72 | 0.76 |
| F78W | 51.3 | 74.0 | 64.3 | 59.1 | 117.6 | 0.80 | 0.45 | 0.57 | 0.61 | 0.75 | 0.73 | 0.79 |
| H79F | 54.8 | 79.0 | 68.5 | 53.5 | 100.5 | 0.68 | 0.50 | 0.58 | 0.57 | 0.69 | 0.81 | 0.88 |
| H79M | 51.2 | 73.9 | 64.2 | 71.6 | 126.9 | 0.97 | 0.60 | 0.74 | 0.80 | 0.90 | 0.86 | 0.90 |
| H79N | 49.0 | 70.5 | 61.2 | 77.2 | 142.1 | 1.10 | 0.62 | 0.77 | 0.73 | 0.81 | 0.89 | 0.94 |
| H79P | 41.9 | 60.4 | 52.4 | 60.7 | 142.0 | 1.00 | 0.44 | 0.55 | 0.63 | 0.78 | 0.73 | 0.79 |
| H79Q | 46.3 | 66.7 | 57.9 | 70.5 | 133.6 | 1.06 | 0.42 | 0.53 | 0.59 | 0.72 | 0.76 | 0.82 |
| H79S | 41.2 | 59.5 | 51.6 | 59.9 | 127.6 | 1.01 | 0.46 | 0.58 | 0.64 | 0.79 | 0.81 | 0.88 |
| H79V | 42.2 | 60.8 | 52.7 | 52.0 | 118.3 | 0.85 | 0.34 | 0.40 | 0.60 | 0.73 | 0.80 | 0.87 |
| L80E | 35.7 | 51.3 | 44.5 | 56.6 | 117.6 | 1.10 | 0.43 | 0.54 | 0.72 | 0.80 | 0.81 | 0.85 |
| L80F | 40.4 | 58.1 | 50.4 | 83.2 | 137.0 | 1.43 | 0.60 | 0.75 | 0.84 | 0.95 | 1.00 | 1.05 |
| L80Y | 50.6 | 72.8 | 63.1 | 89.1 | 110.1 | 1.23 | 0.49 | 0.61 | 0.66 | 0.81 | 0.81 | 0.87 |
| R81E | 46.7 | 67.3 | 58.4 | 66.2 | 124.3 | 0.98 | 0.50 | 0.58 | 0.66 | 0.79 | 0.82 | 0.89 |
| R81L | 40.4 | 58.2 | 50.5 | 63.7 | 107.0 | 1.10 | 0.60 | 0.75 | 0.70 | 0.79 | 0.83 | 0.87 |
| R81M | 42.8 | 61.7 | 53.5 | 70.5 | 107.8 | 1.14 | 0.59 | 0.73 | 0.65 | 0.72 | 0.88 | 0.92 |
| R81N | 36.2 | 52.2 | 45.3 | 67.3 | 100.0 | 1.29 | 0.41 | 0.52 | 0.57 | 0.70 | 0.70 | 0.76 |
| R81P | 44.6 | 64.3 | 55.8 | 80.7 | 113.7 | 1.26 | 0.47 | 0.60 | 0.67 | 0.82 | 0.75 | 0.81 |
| R81T | 49.5 | 71.3 | 61.9 | 80.4 | 128.3 | 1.13 | 0.54 | 0.63 | 0.68 | 0.82 | 0.83 | 0.90 |
| N88H | 30.4 | 38.7 | 36.9 | 56.3 | 97.9 | 1.45 | 0.38 | 0.43 | 0.59 | 0.62 | 0.73 | 0.87 |
| Q126I | 45.9 | 51.9 | 50.0 | 78.2 | 95.9 | 1.51 | 0.60 | 0.76 | 0.70 | 0.85 | 0.86 | 0.95 |

A secondary analysis was performed for IL-2 mutein preparations quantitated at <0.066 ng/μl. This analysis identified additional IL-2 muteins that also exhibited TNF-alpha production less than 96.2% of that exhibited by the des-alanyl-1 C125S human IL-2 mutein, each at 1 nM, and which maintained NK cell proliferation at least 100% of that induced by this reference IL-2 molecule when at 1 nM concentration. See Table 7.

TABLE 7
IL-2 muteins identified using the following selection criteria: TNF-α production <96.2% of that
observed for des-alanyl-1, C125S human IL-2 (Pro), each at 1.0 nM, and NK cell proliferation at 1.0 nM
maintained or improved relative to des-alanyl-1, C125S human IL-2 (Pro). Total TNF-α production
(pg/ml) at 1.0 nM and TNF-α production as a percentage of that observed for des-alanyl-1, C125S human
IL-2 (% Pro) or C125S human IL-2 (% Ala-Pro) are shown. CPA values are expressed as a percentage of
that observed for des-alanyl-1, C125S human IL-2 (% Pro). The ratio of % NK cell proliferation at 0.1 nM
protein relative to % TNF-α production at 1.0 nM protein is shown (% CPA 0.1:TNF-α). Cytotoxicity assay
values are expressed as a ratio of the values observed for des-alanyl-1, C125S human IL-2 (:Pro) or for
C125S human IL-2 (:Ala-Pro).

| | | | | | | | Cytotoxicity Assay | | | | | |
|---|---|---|---|---|---|---|---|---|---|---|---|---|
| | | | TNF-α | CPA | CPA | | | | | | ADCC | |
| | TNF-α | TNF-α | % Ala- | % Pro | % Pro | % CPA | NK (K562) | | LAK (Daudi) | | (Daudi + Ritux) | |
| Mutation | pg/ml | % Pro | Pro | 0.1 nM | 1 nM | 0.1:TNF-α | :Pro | :Ala-Pro | :Pro | :Ala-Pro | :Pro | :Ala-Pro |
| E61M | 24.9 | 32.8 | 34.2 | 56.6 | 98.4 | 1.72 | 0.39 | 0.51 | 0.51 | 0.59 | 0.57 | 0.67 |
| E62T | 26.3 | 34.6 | 36.1 | 69.9 | 96.9 | 2.02 | 0.71 | 0.93 | 0.66 | 0.77 | 0.52 | 0.61 |
| E62Y | 35.0 | 46.1 | 47.9 | 92.0 | 99.9 | 2.00 | 0.40 | 0.46 | 0.74 | 0.82 | 0.68 | 0.77 |
| L80G | 35.5 | 51.1 | 44.3 | 74.5 | 128.0 | 1.46 | 0.45 | 0.56 | 0.59 | 0.72 | 0.73 | 0.79 |
| L80N | 21.7 | 31.4 | 27.2 | 38.5 | 101.2 | 1.23 | 0.32 | 0.37 | 0.56 | 0.68 | 0.78 | 0.85 |
| L80R | 66.9 | 96.2 | 83.4 | 162.5 | 102.2 | 1.69 | 0.85 | 1.05 | 0.85 | 0.96 | 0.82 | 0.86 |
| L80W | 33.4 | 48.1 | 41.7 | 61.8 | 121.5 | 1.29 | 0.41 | 0.52 | 0.59 | 0.73 | 0.76 | 0.82 |
| D84R | 30.0 | 35.7 | 34.7 | 37.8 | 95.4 | 1.06 | 0.41 | 0.45 | 0.45 | 0.52 | 0.54 | 0.62 |
| E95M | 48.4 | 44.0 | 39.6 | 49.5 | 118.0 | 1.12 | 0.36 | 0.49 | 0.27 | 0.36 | 0.44 | 0.52 |
| Y107L | 47.5 | 54.3 | 55.2 | 68.9 | 116.4 | 1.27 | 0.55 | 0.76 | 0.52 | 0.70 | 0.70 | 0.85 |
| Y107Q | 50.5 | 57.6 | 58.6 | 74.3 | 120.0 | 1.29 | 0.58 | 0.75 | 0.53 | 0.78 | 0.69 | 0.87 |
| Y107T | 47.6 | 54.3 | 55.2 | 62.9 | 115.1 | 1.16 | 0.31 | 0.41 | 0.33 | 0.50 | 0.68 | 0.78 |
| N88T | 31.4 | 39.8 | 38.1 | 48.7 | 94.2 | 1.22 | 0.40 | 0.45 | 0.53 | 0.56 | 0.68 | 0.73 |

### Example 4

### Identification of IL-2 Muteins with Enhanced NK-Mediated Cytotoxicity

Muteins were selected that enhanced NK-mediated cytotoxicity against K562 cells at least 140% over that of the C125S human IL-2 mutein (i.e., Ala-Pro) and at least 115% over that of the des-alanyl-1 C125S human IL-2 mutein (i.e., mutein present in Proleukin® IL-2) when assayed at either 0.1 nM or 1.0 nM, as well as eliciting less than 100% of the TNF-α production exhibited by either of these two reference IL-2 muteins when assayed at 1 nM, and maintaining NK cell proliferation (at least 100%) relative to these two reference IL-2 muteins when assayed at 0.1 nM or 1 nM. See Table 8.

TABLE 8
IL-2 mutein natural cytotoxicity muteins identified using NK3.3 cytotoxicity assay (K562 targets). Total
TNF-α production (pg/ml) at 1.0 nM and TNF-α production as a percentage of that observed for des-alanyl-1,
C125S human IL-2 (% Pro) or C125S human IL-2 (% Ala-Pro) are shown. CPA values are expressed as a percentage of that
observed for des-alanyl-1, C125S human IL-2 (% Pro) or C125S human IL-2 (% Ala-Pro) at 0.1 nM or 1 nM. The ratio of
% NK cell proliferation at 0.1 nM protein relative to % TNF-α production at 1.0 nM protein is shown for the Pro
(% CPA:TNF-α (Pro)) and Ala-Pro (% CPA:TNF-α (Ala-Pro)). Cytotoxicity assay values are expressed as
a ratio of the values observed for des-alanyl-1, C125S human IL-2 (:Pro) or for C125S human IL-2 (:Ala-Pro).

| | | | | | | | | | | Cytotoxicity Assay | | | | | |
|---|---|---|---|---|---|---|---|---|---|---|---|---|---|---|---|
| | | | | | | | | | | | | | | ADCC | |
| | | | TNF- | | CPA | | CPA | | | | | LAK | | (Daudi + | |
| | TNF- | TNF- | α | CPA | % Ala- | CPA | % Ala- | % CPA:TNF- | % CPA:TNF- | NK (K562) | | (Daudi) | | Ritux) | |
| | α | α % | % Ala- | % Pro | Pro | % Pro | Pro | α | α | | :Ala- | | :Ala- | | :Ala- |
| Mutation | pg/ml | Pro | Pro | 0.1 nM | 0.1 nM | 1 nM | 1 nM | (Pro) | (Ala-Pro) | :Pro | Pro | :Pro | Pro | :Pro | Pro |
| P34R | 50.8 | 98.5 | 95.4 | 124.3 | 107.0 | 107.4 | 108.5 | 1.26 | 1.12 | 1.16 | 1.43 | 0.80 | 0.97 | 0.90 | 0.98 |
| P34T | 51.6 | 100.1 | 97.0 | 123.5 | 106.3 | 110.8 | 111.9 | 1.23 | 1.10 | 1.29 | 1.59 | 0.87 | 1.05 | 0.92 | 1.00 |
| L36A | 37.9 | 72.8 | 70.5 | 123.9 | 106.6 | 115.5 | 116.7 | 1.70 | 1.51 | 1.23 | 1.51 | 0.98 | 1.18 | 0.95 | 1.03 |
| L36D | 38.6 | 74.1 | 71.7 | 128.8 | 110.9 | 108.4 | 109.4 | 1.74 | 1.55 | 1.16 | 1.43 | 0.95 | 1.14 | 1.02 | 1.10 |
| L36P | 40.1 | 76.8 | 74.4 | 132.7 | 114.2 | 113.8 | 115.0 | 1.73 | 1.54 | 1.24 | 1.52 | 1.04 | 1.26 | 0.95 | 1.03 |
| R38P | 45.8 | 88.8 | 86.0 | 135.8 | 116.9 | 101.3 | 102.3 | 1.53 | 1.36 | 1.17 | 1.44 | 0.87 | 1.05 | 0.91 | 0.99 |
| F42A | 40.9 | 79.1 | 76.6 | 116.2 | 100.0 | 100.1 | 101.1 | 1.47 | 1.31 | 1.23 | 1.54 | 1.10 | 1.31 | 0.94 | 1.05 |
| L80R | 56.0 | 98.1 | 93.5 | 135.4 | 108.6 | 101.6 | 101.4 | 1.38 | 1.16 | 1.21 | 1.63 | 1.06 | 1.36 | 1.02 | 1.11 |

### Example 5

### Identification of IL-2 Muteins with Enhanced LAK Activity

Muteins were then selected based on the following critera: enhanced NK cell-mediated LAK activity to greater than 120% that of the C125S human IL-2 mutein (i.e., Ala-Pro) and maintained (at least 100%) NK cell-mediated LAK activity relative to the des-alanyl-1, C125 S human IL-2 mutein present in Proleukin® IL-2, as well as eliciting less than 100% the TNF-α of both the des-alanyl-1, C125S human IL-2 mutein and the C125S human IL-2 mutein at 1 nM and maintaining NK cell proliferation (at least 100%) compared to both of these reference IL-2 muteins at both 0.1 nM and 1 nM. See Table 9.

TABLE 9
IL-2 mutein lymphokine activated killer (LAK) activity hits identified using NK3.3
cytotoxicity assay (Daudi targets). Total TNF-α production (pg/ml) and TNF-α production as a
percentage of that observed for des-alanyl-1, C125S human IL-2 (% Pro) or C125S human IL-2
(% Ala-Pro) are shown for each individual mutein. CPA values are expressed as a percentage of
that observed for des-alanyl-1, C125S human IL-2 (% Pro) or C125S human IL-2 (% Ala-Pro) at
0.1 nM or 1 nM. The ratio of % NK cell proliferation at 0.1 nM protein relative to % TNF-α
production at 1.0 nM protein is shown for the Pro (% CPA:TNF-α (Pro)) and Ala-Pro
(% CPA:TNF-α (Ala-Pro)). Cytotoxicity assay values are expressed as a ratio of the values
observed for des-alanyl-1, C125S human IL-2 (:Pro) or for C125S human IL-2 (:Ala-Pro).

| | | | | | | | | | | | | | | ADCC | |
|---|---|---|---|---|---|---|---|---|---|---|---|---|---|---|---|
| | | | | | CPA | | CPA | | | | | LAK | | (Daudi + | |
| | TNF- | TNF- | TNF- | CPA | % Ala- | CPA | % Ala- | | | NK (K562) | | (Daudi) | | Ritux) | |
| | α | α % | α % | % Pro | Pro | % Pro | Pro | % CPA:TNF α | % CPA:TNF-α | | :Ala- | | :Ala- | | :Ala- |
| Mutation | pg/ml | Pro | Ala-Pro | 0.1 nM | 0.1 nM | 1 nM | 1 nM | Pro | Ala-Pro | :Pro | Pro | :Pro | Pro | :Pro | Pro |
| L36P | 40.1 | 76.8 | 74.4 | 132.7 | 114.2 | 113.8 | 115.0 | 1.73 | 1.54 | 1.24 | 1.52 | 1.04 | 1.26 | 0.95 | 1.03 |
| L36R | 43.8 | 84.0 | 81.4 | 128.3 | 110.4 | 116.7 | 117.9 | 1.53 | 1.36 | 1.07 | 1.32 | 1.04 | 1.25 | 1.05 | 1.13 |
| F42A | 40.9 | 79.1 | 76.6 | 116.2 | 100.0 | 100.1 | 101.1 | 1.47 | 1.31 | 1.23 | 1.54 | 1.10 | 1.31 | 0.94 | 1.05 |
| L80R | 56.0 | 98.1 | 93.5 | 135.4 | 108.6 | 101.6 | 101.4 | 1.38 | 1.16 | 1.21 | 1.63 | 1.06 | 1.36 | 1.02 | 1.11 |
| V91Q | 49.0 | 93.4 | 87.9 | 130.3 | 106.8 | 101.6 | 102.1 | 1.40 | 1.22 | 0.77 | 1.07 | 1.04 | 1.22 | 0.93 | 1.07 |

### Example 6

### Identification of IL-2 Muteins with Enhanced ADCC Activity

Muteins were then selected based on the criteria of having enhanced NK cell-mediated ADCC activity at least 115% that of the C125S human IL-2 mutein (Ala-Pro) and at least 105% that of the des-alanyl-1, C125S human IL-2 mutein (Pro), and that elicited less than 100% the TNF-α of both of the reference IL-2 muteins, each at 1 nM, and maintained NK cell proliferation (at least 100%) compared to both of the reference IL-2 muteins, each at 0.1 nM. See Table 10.

TABLE 10
IL-2 mutein antibody dependent cellular cytotoxicity (ADCC) hits identified using NK3.3 cytotoxicity assay (rituximab-
coated Daudi targets). Total TNF-α production (pg/ml) and TNF-α production as a percentage of that observed for des-alanyl-1,
C125S human IL-2 (% Pro) or C125S human IL-2 (% Ala-Pro) are shown. CPA values are expressed as a percentage of that observed
for des-alanyl-1, C125S human IL-2 (% Pro). The ratio of % NK cell proliferation at 0.1 nM protein relative to % TNF-α production at
1.0 nM protein is shown (% CPA:TNF-α). Cytotoxicity assay values are expressed as a ratio of the values observed for des-alanyl-1,
C125S human IL-2 (:Pro) or for C125S human IL-2 (:Ala-Pro).

| | | | | | | | | | | Cytotoxicity Assays | | | | | |
|---|---|---|---|---|---|---|---|---|---|---|---|---|---|---|---|
| | | | | | | | | | | | | | | ADCC | |
| | | | TNF- | | CPA | | CPA | | | NK | | LAK | | (Daudi + | |
| | TNF- | TNF- | α % | CPA | % Ala- | CPA | % Ala- | | % CPA:TNF-α | (K562) | | (Daudi) | | Ritux) | |
| | α | α % | Ala- | % Pro | Pro | % Pro | Pro | % CPA:TNF-α | (Ala- | | :Ala- | | :Ala- | | :Ala- |
| Mutation | pg/ml | Pro | Pro | 0.1 nM | 0.1 nM | 1 nM | 1 nM | (Pro) | Pro) | :Pro | Pro | :Pro | Pro | :Pro | Pro |
| D20E | 45.8 | 88.4 | 85.6 | 130.8 | 112.6 | 93.5 | 94.5 | 1.48 | 1.32 | 0.90 | 1.13 | 0.94 | 1.12 | 1.07 | 1.19 |
| E67A | 44.1 | 77.3 | 73.6 | 128.1 | 102.7 | 106.4 | 106.4 | 1.66 | 1.40 | 0.93 | 1.26 | 0.94 | 1.13 | 1.11 | 1.18 |

### Example 7

### Selection of Muteins Supporting Enhanced NK Cell Survival

Muteins were screened for their ability to enhance NK cell survival as compared to the des-alanyl-1, C125S human IL-2 mutein. See Table 11.

TABLE 11
IL-2 mutein cell survival positive hits identified using
NK3.3 pAKT induction assay.

| IL-2 MUTEIN | pAKT | IL-2 MUTEIN | pAKT |
|---|---|---|---|
| 2 nM | (U/ml) | 2 nM | (U/ml) |
| Proleukin | 27.04 | Proleukin | 27.04 |
| T7D | 29.1 | L80R | 31.71 |
| K9D | 29.85 | L80T | 32.87 |
| K9R | 27.96 | L80V | 35.89 |
| K9V | 28.44 | L80W | 34.67 |
| E15A | 32.27 | R81K | 36.08 |
| I24L | 31.67 | R81M | 28.89 |
| N33E | 36.92 | R81N | 28.58 |
| L36I | 27.09 | R81P | 27.35 |
| L36K | 28.34 | R81T | 31.39 |
| L36R | 30.22 | S87T | 27.66 |
| R38P | 29.47 | V91W | 29.7 |
| L40D | 28.72 | L94A | 29.5 |
| L40G | 30.49 | L94T | 31.29 |
| L40N | 31.13 | L94V | 34.95 |
| T41E | 28.91 | L94Y | 29.19 |
| H79M | 27.23 | E95D | 29.8 |
| H79P | 27.05 | T102S | 33.81 |
| H79Q | 27.85 | T102V | 27.04 |
| H79S | 29.24 | M104G | 32.95 |
| H79V | 27.32 | E106K | 28.89 |
| L80E | 30.69 | E116G | 31.29 |
| L80G | 28.54 | N119Q | 33.14 |
| L80K | 28.28 | T123C | 34.67 |
| L80N | 27.85 | | |

### Example 8

### Selection of Human IL-2 Muteins with Most Improved Therapeutic Profile

Using the selection criteria described above, twenty-five human IL-2 muteins were identified as being of particular interest. These muteins are shown in Table 12.

TABLE 12
Top twenty-five human IL-2 muteins identified in screening process.

| | TNF-α | | | | | TNF- :Ala- | | | | |
|---|---|---|---|---|---|---|---|---|---|---|
| | pg/ml | pg/ml | pg/ml | pg/ml | pg/ml | α Pro | | | | |
| Seq | 5 pM | 20 pM | 50 pM | 100 pM | 1000 pM | 5 pM | 20 pM | 50 pM | 100 pM | 1000 pM |
| H16D | 6.2 | 26.1 | 32.0 | 34.4 | 139.7 | 0.85 | 2.02 | 1.35 | 0.88 | 0.77 |
| L19D | 6.2 | 10.2 | 18.4 | 22.1 | 80.2 | 0.85 | 0.79 | 0.77 | 0.56 | 0.44 |
| L19E | 7.6 | 18.7 | 35.5 | 39.7 | 175.7 | 1.03 | 1.45 | 1.49 | 1.01 | 0.97 |
| L36D | 4.9 | 15.7 | 23.1 | 32.6 | 109.9 | 0.66 | 1.21 | 0.97 | 0.83 | 0.61 |
| L36P | 3.1 | 7.8 | 25.8 | 27.4 | 133.7 | 0.42 | 0.60 | 1.09 | 0.70 | 0.74 |
| L40D | 6.2 | 17.5 | 21.8 | 42.0 | 165.1 | 0.85 | 1.35 | 0.91 | 1.07 | 0.91 |
| L40G | 6.2 | 12.6 | 23.1 | 36.1 | 149.4 | 0.85 | 0.98 | 0.97 | 0.92 | 0.82 |
| F42E | 6.7 | 4.2 | 11.7 | 9.5 | 77.3 | 0.91 | 0.33 | 0.49 | 0.24 | 0.43 |
| F42R | 4.9 | 8.4 | 16.4 | 24.4 | 98.0 | 0.66 | 0.65 | 0.69 | 0.62 | 0.54 |
| E61R | 6.7 | 10.8 | 19.7 | 26.2 | 92.8 | 0.91 | 0.84 | 0.83 | 0.67 | 0.51 |
| P65L | ND | 5.4 | 9.5 | 19.6 | 106.3 | ND | 0.49 | 0.48 | 0.58 | 0.61 |
| P65Y | 3.1 | 10.2 | 19.1 | 26.8 | 109.9 | 0.42 | 0.79 | 0.80 | 0.68 | 0.61 |
| E67A | ND | 8.3 | 13.4 | 22.3 | 143.9 | ND | 0.75 | 0.68 | 0.66 | 0.83 |
| L72N | 5.3 | 3.1 | 15.0 | 23.3 | 94.3 | 0.72 | 0.24 | 0.63 | 0.59 | 0.52 |
| L80K | 6.7 | 16.3 | 32.0 | 49.7 | 150.2 | 0.91 | 1.26 | 1.35 | 1.27 | 0.83 |
| L80V | ND | 9.3 | 15.1 | 27.1 | 170.4 | ND | 0.85 | 0.76 | 0.81 | 0.98 |
| R81K | 5.3 | 14.4 | 27.2 | 30.3 | 189.9 | 0.72 | 1.12 | 1.14 | 0.77 | 1.05 |
| N88D | 3.5 | 4.2 | 5.8 | 8.9 | 53.8 | 0.48 | 0.33 | 0.25 | 0.23 | 0.30 |
| V91D | 7.6 | 7.2 | 11.1 | 10.0 | 67.0 | 1.03 | 0.56 | 0.46 | 0.26 | 0.37 |
| L94Y | ND | 9.3 | 13.2 | 26.0 | 136.2 | ND | 0.85 | 0.67 | 0.77 | 0.79 |
| E95D | ND | 11.7 | 19.1 | 34.2 | 152.2 | ND | 1.07 | 0.97 | 1.02 | 0.88 |
| E95G | 7.1 | 13.2 | 21.8 | 21.0 | 103.2 | 0.97 | 1.02 | 0.91 | 0.53 | 0.57 |
| V91N | 9.0 | 15.1 | 21.8 | 25.0 | 150.9 | 1.22 | 1.16 | 0.91 | 0.64 | 0.83 |
| Y107H | 9.0 | 18.1 | 30.6 | 38.5 | 135.2 | 1.22 | 1.40 | 1.29 | 0.98 | 0.75 |
| Y107R | 7.6 | 15.7 | 27.2 | 26.2 | 116.6 | 1.03 | 1.21 | 1.14 | 0.67 | 0.64 |

| | | | | | | | | | | | NK: |
|---|---|---|---|---|---|---|---|---|---|---|---|
| | | | | | | KIT:Ala- | | | | | Ala- |
| | :Ala- | | | | | 225 MTT Pro | | | | | Pro |
| | NK92 MTT Pro | | | | | | | | 100 | 500 | 100 |
| Seq | 5 pM | 20 pM | 50 pM | 100 pM | 1000 pM | 5 pM | 20 pM | 50 pM | pM | pM | pM |
| H16D | 0.93 | 1.99 | 2.68 | 2.04 | 1.04 | 1.01 | 1.11 | 1.21 | 1.13 | 1.18 | 1.10 |
| L19D | 0.94 | 1.70 | 2.26 | 1.85 | 1.13 | 0.95 | 1.07 | 1.13 | 1.21 | 1.14 | 0.83 |
| L19E | 1.08 | 1.72 | 2.12 | 1.73 | 0.95 | 0.99 | 1.11 | 1.13 | 1.14 | 1.20 | 0.86 |
| L36D | 1.11 | 1.34 | 2.07 | 1.90 | 1.05 | 0.91 | 1.03 | 1.08 | 1.15 | 1.15 | 1.04 |
| L36P | 0.79 | 0.95 | 1.12 | 1.10 | 1.07 | 0.96 | 1.02 | 1.00 | 1.08 | 1.10 | 0.86 |
| L40D | 1.28 | 1.37 | 1.57 | 2.04 | 0.99 | 0.97 | 1.06 | 1.04 | 1.14 | 1.15 | 1.21 |
| L40G | 1.21 | 1.28 | 1.33 | 1.53 | 1.05 | 1.02 | 1.07 | 1.07 | 1.16 | 1.13 | 1.18 |
| F42E | 0.62 | 0.37 | 0.60 | 0.72 | 1.09 | 0.84 | 0.79 | 0.76 | 0.88 | 1.01 | 0.91 |
| F42R | 0.67 | 0.94 | 1.71 | 1.58 | 1.06 | 0.83 | 0.84 | 0.87 | 0.94 | 0.99 | 0.80 |
| E61R | 1.07 | 0.90 | 2.14 | 1.89 | 1.07 | 0.94 | 1.05 | 1.14 | 1.20 | 1.17 | 0.96 |
| P65L | 0.38 | 0.73 | 0.80 | 0.37 | 1.34 | 0.71 | 0.76 | 0.79 | 0.88 | 0.79 | 0.47 |
| P65Y | 0.97 | 0.90 | 1.03 | 1.15 | 1.05 | 0.91 | 0.97 | 0.98 | 1.02 | 1.03 | 1.05 |
| E67A | 0.87 | 0.65 | 0.65 | 0.94 | 1.00 | 0.83 | 0.82 | 0.73 | 0.78 | 0.92 | 1.26 |
| L72N | 0.90 | 0.72 | 0.98 | 1.21 | 1.19 | 0.93 | 0.98 | 0.98 | 1.06 | 1.11 | 0.87 |
| L80K | 1.46 | 1.99 | 2.62 | 2.43 | 1.02 | 1.03 | 1.16 | 1.20 | 1.19 | 1.13 | 1.01 |
| L80V | 0.81 | 0.57 | 0.60 | 1.07 | 1.36 | 0.84 | 0.80 | 0.80 | 0.90 | 0.82 | 0.88 |
| R81K | 0.97 | 0.99 | 1.02 | 1.10 | 1.00 | 0.98 | 0.97 | 1.03 | 1.03 | 1.14 | 1.07 |
| N88D | 0.82 | 0.33 | 0.25 | 0.30 | 1.17 | 0.85 | 0.77 | 0.73 | 0.79 | 1.09 | 0.82 |
| V91D | 0.83 | 0.41 | 0.82 | 0.88 | 1.22 | 0.89 | 0.91 | 0.95 | 1.05 | 1.06 | 0.89 |
| L94Y | 0.91 | 0.67 | 0.71 | 1.08 | 0.91 | 0.93 | 0.94 | 0.89 | 0.95 | 0.87 | 0.87 |
| E95D | 1.23 | 1.04 | 1.17 | 1.10 | 1.37 | 0.85 | 0.85 | 0.77 | 0.88 | 0.85 | 0.98 |
| E95G | 1.02 | 0.98 | 1.26 | 1.72 | 1.14 | 1.01 | 1.08 | 1.02 | 1.11 | 1.09 | 1.08 |
| V91N | 1.20 | 0.93 | 0.96 | 0.95 | 0.98 | 0.97 | 1.06 | 1.04 | 1.05 | 1.05 | 1.13 |
| Y107H | 1.37 | 1.92 | 2.65 | 1.98 | 0.98 | 1.05 | 1.19 | 1.18 | 1.16 | 1.09 | 1.16 |
| Y107R | 1.15 | 2.22 | 3.16 | 2.21 | 1.04 | 0.99 | 1.16 | 1.15 | 1.18 | 1.14 | 0.98 |

After selection of the muteins based on the above criteria, the muteins were further divided into groups that satisfied the following selection criteria:

1) muteins that exhibit TNF-α production <80% of that observed for the C125S human IL-2 mutein and that:
a) maintain proliferation at 1 nM, but relative to the reference IL-2 mutein, proliferative activity drops at lower concentrations, which includes the des-alanyl-1, C125S human IL-2 mutein or the C125S human IL-2 mutein further comprising the F42E or V91D mutation (see Table 13); or,
b) exhibit significant decreases in TNF-α production at 1 nM, and where proliferative activity is maintained down to 50 pM, which includes the des-alanyl-1, C125S human IL-2 mutein or the C125S human IL-2 mutein further comprising the L72N mutation (see Table 14);

2) muteins that augment NK-92 proliferation >200% compared to C125S human IL-2 mutein at one or more concentrations tested (5 pM, 20 pM, 50 pM, 100 pM, and 1000 pM) without deleterious impact on TNF-α production (<100% TNF-α production relative to that observed for the reference IL-2 mutein at a concentration of 100 pM or 1 nM). Furthermore, selection criteria included a proliferation index greater than 150% of that observed for the reference IL-2 mutein, i.e., C125S human IL-2 (Ala-Pro) for at least 2 concentrations tested; this group includes the des-alanyl-1, C125S human IL-2 mutein or the C125S human IL-2 mutein further comprising the L36D or L40D mutation (see Table 15); and,

3) muteins that showed increased proliferative activity and decreased TNF-a production, where TNF-α production is <75% of that observed for the C125S human IL-2 mutein when tested at 1 nM, and proliferation of NK cells is >150% of that observed for the C125S human IL-2 mutein at any one concentration tested (5 pM, 20 pM, 50 pM, 100 pM, and 1000 pM); this group includes the des-alanyl-1, C125S human IL-2 mutein or the C125S human IL-2 mutein further comprising the L19D, F42R, or E61R mutation (see Table 16).

TABLE 13
IL-2 muteins having greatly reduced TNF-α production with NK proliferation maintained at 1.0 nM.

| | | | | | | TNF-α | | | | | NK92 MTT | | | | |
|---|---|---|---|---|---|---|---|---|---|---|---|---|---|---|---|
| | TNF-α | | | | | | :Ala- | :Ala- | :Ala- | :Ala- | | | | | :Ala- |
| | | pg/ml | Pg/ml | pg/ml | pg/ml | :Ala- | Pro | Pro | Pro | Pro | | | :Ala- | :Ala- | Pro |
| | Pg/ml | 20 | 50 | 100 | 1000 | Pro | 20 | 50 | 100 | 1000 | :Ala-Pro | :Ala-Pro | Pro | Pro | 1000 |
| Sequence | 5 pM | pM | pM | pM | pM | 5 pM | pM | pM | pM | pM | 5 pM | 20 pM | 50 pM | 100 pM | pM |
| V91D | 7.6 | 7.2 | 11.1 | 10.0 | 67.0 | 1.03 | 0.56 | 0.46 | 0.26 | 0.37 | 0.83 | 0.41 | 0.82 | 0.88 | 1.22 |
| F42E | 6.7 | 4.2 | 11.7 | 9.5 | 77.3 | 0.91 | 0.33 | 0.49 | 0.24 | 0.43 | 0.62 | 0.37 | 0.60 | 0.72 | 1.09 |

TABLE 14
IL-2 muteins having greatly reduced TNF-α production with NK proliferation maintained at 50 pM.

| | | | | | | | | | | | NK92 | | | | |
|---|---|---|---|---|---|---|---|---|---|---|---|---|---|---|---|
| | | | | | | | | | | | MTT | | | | |
| | TNF-α | | | | | | | | | | | :Ala- | :Ala- | :Ala- | :Ala- |
| | | pg/ml | | | | TNF-α | | | | | :Ala- | Pro | Pro | Pro | Pro |
| Se- | pg/ml | 20 | pg/ml | pg/ml | pg/ml | :Ala-Pro | :Ala-Pro | :Ala-Pro | :Ala-Pro | :Ala-Pro | Pro | 20 | 50 | 100 | 1000 |
| quence | 5 pM | pM | 50 pM | 100 pM | 1000 pM | 5 pM | 20 pM | 50 pM | 100 pM | 1000 pM | 5 pM | pM | pM | pM | pM |
| L72N | 5.3 | 3.1 | 15.0 | 23.3 | 94.3 | 0.72 | 0.24 | 0.63 | 0.59 | 0.52 | 0.90 | 0.72 | 0.98 | 1.21 | 1.19 |

TABLE 15
IL-2 muteins inducing strong NK cell proliferation without deleterious impact on TNF-α production.

| | | | | | | | | | | | NK92 | | | | |
|---|---|---|---|---|---|---|---|---|---|---|---|---|---|---|---|
| | | | | | | | | | | | MTT | | | | |
| | TNF-α | | | | | | | | | | | :Ala- | :Ala- | :Ala- | :Ala- |
| | | pg/ml | | | | TNF-α | | | | | :Ala- | Pro | Pro | Pro | Pro |
| Se- | Pg/ml | 20 | pg/ml | pg/ml | pg/ml | :Ala-Pro | :Ala-Pro | :Ala-Pro | :Ala-Pro | :Ala-Pro | Pro | 20 | 50 | 100 | 1000 |
| quence | 5 pM | pM | 50 pM | 100 pM | 1000 pM | 5 pM | 20 pM | 50 pM | 100 pM | 1000 pM | 5 pM | pM | pM | pM | pM |
| L36D | 4.9 | 15.7 | 23.1 | 32.6 | 109.9 | 0.66 | 1.21 | 0.97 | 0.83 | 0.61 | 1.11 | 1.34 | 2.07 | 1.90 | 1.05 |
| L40D | 6.2 | 17.5 | 21.8 | 42.0 | 165.1 | 0.85 | 1.35 | 0.91 | 1.07 | 0.91 | 1.28 | 1.37 | 1.57 | 2.04 | 0.99 |

TABLE 16
IL-2 muteins identified within the bifunctional series - increased NK proliferation and decreased
TNF-α relative to C125S human IL-2 mutein.

| | | | | | | | | | | | NK92 | | | | |
|---|---|---|---|---|---|---|---|---|---|---|---|---|---|---|---|
| | | | | | | | | | | | MTT | | | | |
| | TNF-α | | | | | | | | | | | :Ala- | :Ala- | :Ala- | :Ala- |
| | | pg/ml | | | | TNF-α | | | | | :Ala- | Pro | Pro | Pro | Pro |
| Se- | pg/ml | 20 | pg/ml | pg/ml | pg/ml | :Ala-Pro | :Ala-Pro | :Ala-Pro | :Ala-Pro | :Ala-Pro | Pro | 20 | 50 | 100 | 1000 |
| quence | 5 pM | pM | 50 pM | 100 pM | 1000 pM | 5 pM | 20 pM | 50 pM | 100 pM | 1000 pM | 5 pM | pM | pM | pM | pM |
| L19D | 6.2 | 10.2 | 18.4 | 22.1 | 80.2 | 0.85 | 0.79 | 0.77 | 0.56 | 0.44 | 0.94 | 1.70 | 2.26 | 1.85 | 1.13 |
| F42R | 4.9 | 8.4 | 16.4 | 24.4 | 98.0 | 0.66 | 0.65 | 0.69 | 0.62 | 0.54 | 0.67 | 0.94 | 1.71 | 1.58 | 1.06 |
| E61R | 6.7 | 10.8 | 19.7 | 26.2 | 92.8 | 0.91 | 0.84 | 0.83 | 0.67 | 0.51 | 1.07 | 0.90 | 2.14 | 1.89 | 1.07 |

### Example 9

### Human IL-2 Muteins Maintain T Cell Proliferation

A secondary functional endpoint serving as a basis of selecting beneficial mutations was maintained or improved T cell proliferation by the human Kit225 T cell line relative to that observed with Ala-Pro IL-2 (i.e., C25S human IL-2 mutein) or Proleukin® (i.e., des-alanyl-1, C125S human IL-2 mutein). A subset of the 168 muteins shown in Table 1 above was selected to test for this functional endpoint. Results are shown in Table 17 below.

TABLE 17
Kit 225 human T cell line proliferation - ratio of OD from MTT assay as
compared to Y-Pro IL-2 control (yeast-expressed des-alanyl-1, C125S
human IL-2 mutein) or Pro control (aldesleukin, Proleukin ®).

| | | vs 100 | vs 500 | vs 50 | | |
|---|---|---|---|---|---|---|
| IL-2 | vs 50 pm | pm | pm | pm | vs 100 pm | vs 500 pm |
| Mutein | Y-Pro | Y-Pro | Y-Pro | Pro | Pro | Pro |
| H16D | 1.13 | 1.13 | 1.05 | 1.33 | 1.22 | 1.06 |
| L19D | 0.98 | 1.10 | 1.08 | 1.15 | 1.19 | 1.09 |
| L19E | 0.90 | 1.01 | 1.04 | 1.06 | 1.09 | 1.05 |
| L36D | 1.03 | 1.06 | 1.07 | 1.21 | 1.15 | 1.08 |
| L36E | 1.05 | 1.15 | 1.05 | 1.24 | 1.25 | 1.06 |
| L36P | 0.97 | 1.04 | 1.01 | 1.15 | 1.13 | 1.03 |
| L40D | 1.01 | 1.05 | 1.04 | 1.18 | 1.14 | 1.06 |
| L40G | 0.89 | 1.01 | 1.03 | 1.04 | 1.10 | 1.04 |
| F42E | 0.73 | 0.82 | 1.02 | 0.86 | 0.89 | 1.03 |
| F42R | 1.09 | 1.15 | 1.06 | 1.28 | 1.24 | 1.07 |
| E61R | 1.12 | 1.16 | 1.15 | 1.32 | 1.26 | 1.16 |
| P65H | 1.06 | 1.15 | 1.06 | 1.25 | 1.25 | 1.07 |
| P65L | 0.98 | 1.14 | 1.12 | 1.15 | 1.16 | 1.14 |
| P65Y | 1.17 | 1.14 | 1.10 | 1.37 | 1.24 | 1.12 |
| E67A | 1.11 | 1.11 | 1.08 | 1.31 | 1.21 | 1.10 |
| L72N | 0.88 | 1.00 | 1.07 | 1.03 | 1.09 | 1.08 |
| L80K | 0.75 | 0.87 | 1.01 | 0.88 | 0.94 | 1.02 |
| L80V | 0.91 | 0.99 | 1.05 | 1.07 | 1.08 | 1.07 |
| R81K | 1.00 | 1.03 | 1.06 | 1.17 | 1.12 | 1.07 |
| N88D | 1.31 | 1.21 | 1.15 | 1.54 | 1.32 | 1.16 |
| V91D | 1.24 | 1.20 | 1.13 | 1.45 | 1.30 | 1.14 |
| V91N | 1.13 | 1.12 | 1.10 | 1.33 | 1.22 | 1.11 |
| L94Y | 1.10 | 1.12 | 1.10 | 1.30 | 1.21 | 1.12 |
| E95D | 1.14 | 1.15 | 1.10 | 1.34 | 1.24 | 1.11 |
| E95G | 0.97 | 1.07 | 1.06 | 1.15 | 1.17 | 1.07 |
| Y107H | 1.15 | 1.11 | 1.07 | 1.35 | 1.20 | 1.09 |
| Y107R | 1.16 | 1.13 | 1.07 | 1.36 | 1.23 | 1.08 |
| Y-Pro* | 1.00 | 1.00 | 1.00 | 1.17 | 1.09 | 1.01 |
| Proleukin | 0.85 | 0.92 | 0.99 | 1.00 | 1.00 | 1.00 |
| NO IL-2 | 0.48 | 0.42 | 0.41 | 0.56 | 0.45 | 0.41 |

*Y-Pro = des-alanyl 1, C125S IL-2 expressed in yeast system; all muteins in this assay expressed in yeast vector
Definition of “maintain” T cell proliferation is +/−20% of IL-2 controls

### Example 10

### Identification of Beneficial IL-2 Mutations that Reduce Pro-Inflammatory Cytokine Production while Maintaining or Increasing Levels of Proliferation and Cytotoxicity in Normal Human Peripheral Blood Mononuclear Cells

From the single amino acid substitution series described above, 25 IL-2 muteins were selected for a small-scale expression/purification as indicated in Table 18. These IL-2 muteins were tested for their ability to generate a similar functional profile of increased tolerability and maintained activity in peripheral blood mononuclear cells (PBMC) isolated from several normal human blood donors, as compared to relevant IL-2 controls (des-alanyl-1, C125S human IL-2 mutein (present in Proleukin®) and yeast-expressed C125S human IL-2 mutein (designated Y-Pro in the data herein). Specifically, human PBMC derived from a panel of normal human donors were stimulated with the IL-2 mutein of interest, and assayed for proliferation and pro-inflammatory cytokine production (TNF-α), as well as the ability to kill tumor cell targets by natural/spontaneous cytotoxicity (NK), lymphokine-activated killing (LAK), or antibody dependent cellular cytotoxicity (ADCC).

TABLE 18
Human IL-2 muteins comprising the amino acid sequence
of C125S human IL-2 (SEQ ID NO: 6) or des-alanyl-1, C125S
human IL-2 (SEQ ID NO: 8) with the following additional
substitution were screened for activity in human PBMC.¹

| | | | | |
|---|---|---|---|---|
| H16D | L19D | L19E | L36D | L36P |
| L40D | L40G | F42E | F42R | E61R |
| P65L | P65Y | E67A | L72N | L80K |
| L80V | R81K | N88D | V91D | V91N |
| L94Y | E95D | E95G | Y107H | Y107R |

¹IL-2 muteins identified by: amino acid position relative to mature human IL-2 of SEQ ID NO: 4, and amino acid substitution at that position.

The following primary functional endpoints were used:
1) Reduced pro-inflammatory cytokine production (TNF-α) by human PBMC stimulated with IL-2 mutein as compared to relevant human IL-2 mutein control.
2) Maintained or improved IL-2 induced proliferation in human PBMC without an increase in pro-inflammatory cytokine production as compared to relevant human IL-2 mutein control
3) Maintained or improved NK, LAK, and ADCC mediated cytolytic killing by human PBMC stimulated in vitro with IL-2 mutein as compared to relevant human IL-2 mutein control.
   Assay Descriptions
   Combination Proliferation/Proinflammatory Cytokine Production Assay Procedure

Upon exposure to IL-2, human PBMC proliferate and secrete cytokines in a dose-dependent manner. To maximize data output and efficiency, a combination assay was designed to assess levels of proliferation and cytokine production following 72 h stimulation with the reference IL-2 mutein or the human IL-2 mutein of interest. The assay setup involves isolation of PBMC by density gradient separation (ACDA Vacutainer CPT tubes) from one or more normal human donors. In 96-well tissue-culture treated plates, 200,000 cells per well are incubated with various concentrations of IL-2 (0.039 nM-10 nM) or no IL-2 as a negative control in complete RPMI medium (RPMI, 10% heat-inactivated human AB serum, 25 mM HEPES, 2 mM glutamine, penicillin/streptomycin/fungizone) at 37° C., 7% CO₂. Following 66 h of incubation, an aliquot of cell culture supernatant is removed and frozen for cytokine detection at a later time. The cells are pulsed with 1 μCi ³H-thymidine for 6 h then harvested to determine levels of nucleotide incorporation (Wallac Trilux Microbeta Plate Reader) as a measure of cell proliferation. Commercially available ELISA kits (BioSource International) were used to detect levels of TNF-α in the cell culture supernatants per manufacturer's guidelines. Repeating the assay for a complete panel of six separate donors provides a characterization of representative proliferative and cytokine responses to IL-2 in a “normal population.”

Data Analysis

PBMC samples were plated in duplicate in separate assay plates to assess reproducibility. Proliferation data was analyzed by subtracting background proliferation (PBMC+no IL-2) and means of duplicate samples calculated. Cytokine data was derived from cell culture supernatants removed from assay wells containing PBMC and pooled to obtain the mean cytokine level in the duplicate set up. TNF-α levels were quantitated at pg/ml, based on a standard curve of purified TNF-α contained in the ELISA kit. Data were further compiled for the panel of six normal human donors as outlined in the schematic shown in FIG. 1.

Cytotoxicity Assay (NK/LAK/ADCC)

In this assay, PBMC are separated from whole blood using density gradient centrifugation. PBMC are stimulated for 3 days in the presence of 10 nM IL-2 control or IL-2 mutein of interest, to generate LAK activity as generally practiced in current state of the art (see for example Isolation of Human NK Cells and Generation of LAK activity IN: Current Protocols in Immunology; 1996 John Wiley & Sons, Inc). The resulting cell population contains “effector” cells, which may be classified as NK or LAK, and can kill K562 and Daudi tumor cell targets, respectively. These effector cells may also mediate ADCC, whereby the effector cells recognize the Fc portion of a specific antibody (in this case Rituxan®) that is bound to the Daudi target cells. The assay involves co-incubation of effector cells with calcein AM-labeled target cells at various effector to target cell (E:T ratios) for 4 h. The amount of cytotoxic activity is related to the detection of calcein AM in the culture supernatant. Quantitation is expressed as percent specific lysis at each E:T ratio, based upon determination of spontaneous and maximum release controls. In summary, the assay examines the following biological activities:

| ACTIVITY | EFFECTOR | TARGET | DESCRIPTION |
|---|---|---|---|
| NK | PBMC | K562 | Natural cytotoxicty |
| LAK | PBMC | Daudi | IL-2 activated cells |
| ADCC | PBMC | Daudi + Rituxan | Antibody- |
| | | | dependent |

Data Analysis

Data is obtained from the fluorimeter and expressed in relative fluorescence units (rfu). Controls include labeled target cells alone (min) and labeled target cells with final 1% Triton X-100 as a measure of 100% lysis (max). The percent min to max ratio is calculated using the following equation as a measure of assay validity (assay invalid if >30%):

$$\% min to max = 100 ⨯ \frac{mean spontaneous release rfu}{mean maximum release rfu}$$

Once the assay is deemed valid, the mean and standard deviation for triplicate sample points is calculated, followed by the percent specific lysis from mean of triplicate points using the following equation:

$$\% lysis = 100 ⨯ \frac{\begin{matrix}mean experimental rfu - \\ mean spontaneous release rfu\end{matrix}}{\begin{matrix}mean maximal release rfu - \\ mean spontaneous release rfu\end{matrix}}$$

Data is reported as % specific lysis; in addition the ratio of IL-2 mutein to relevant IL-2 control was used to determine whether cytotoxic activity was maintained relative to control IL-2 in a mixed population of human PBMC donors.
Results

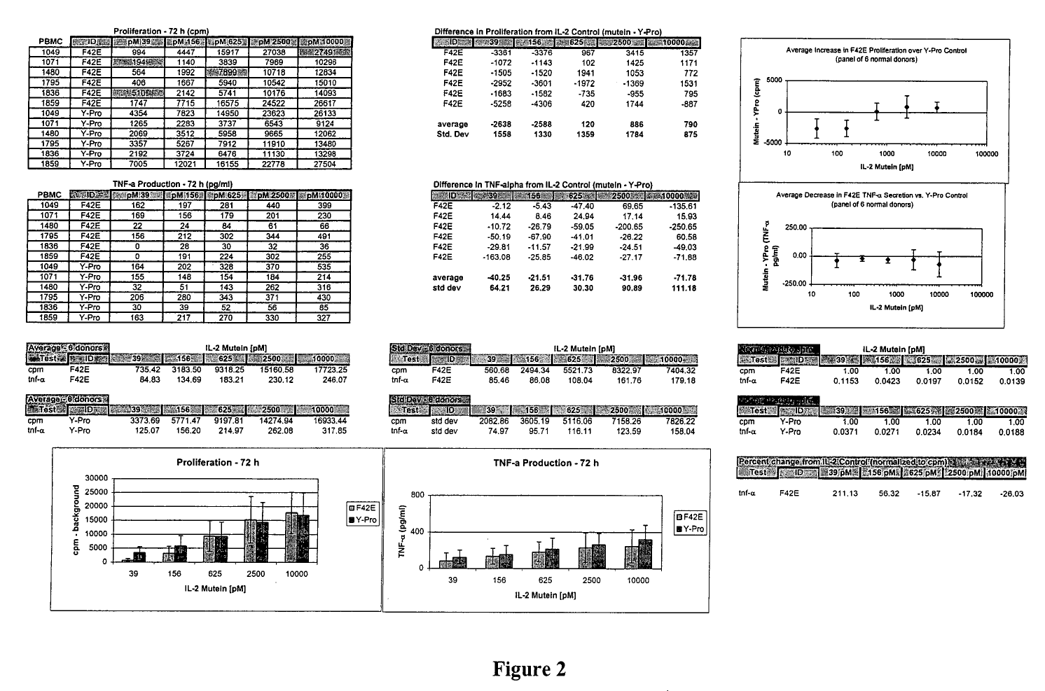

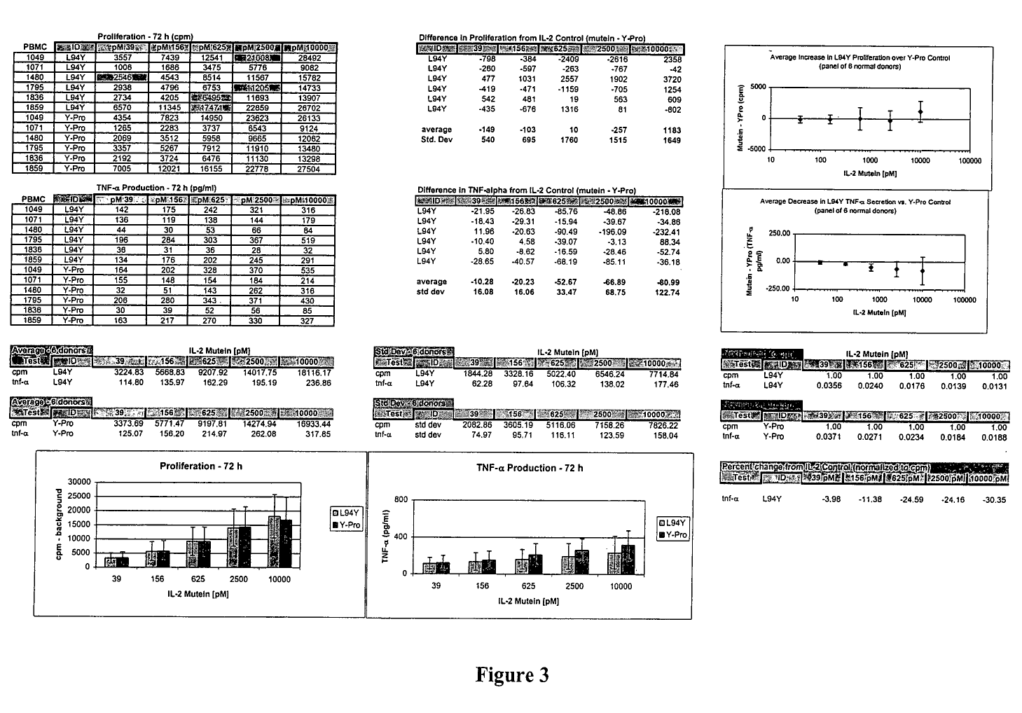

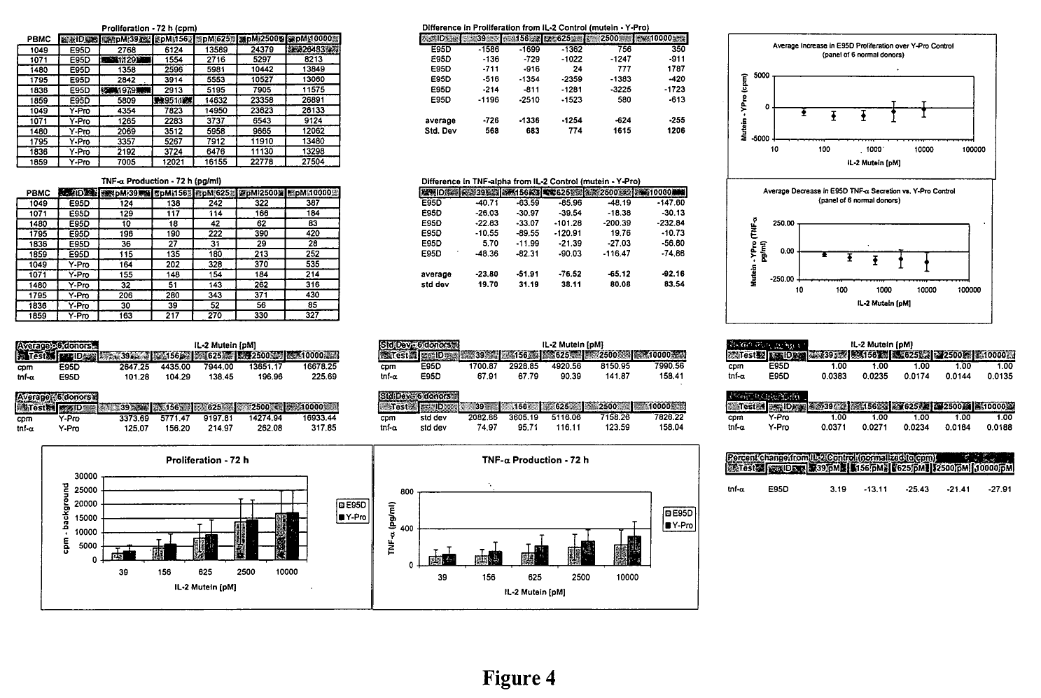

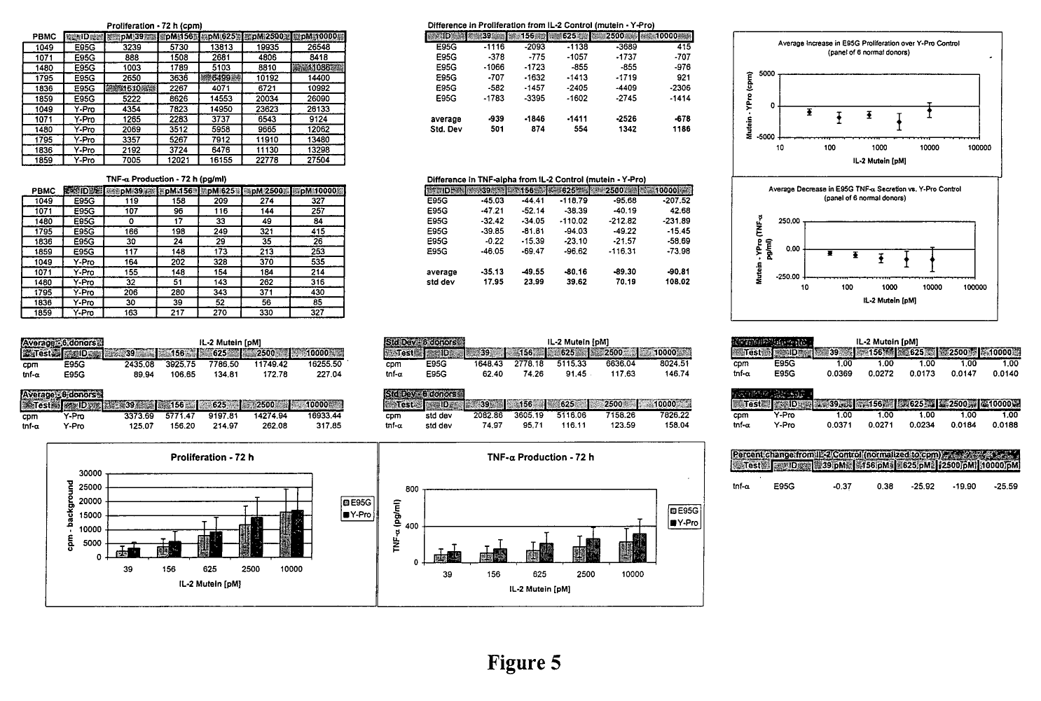

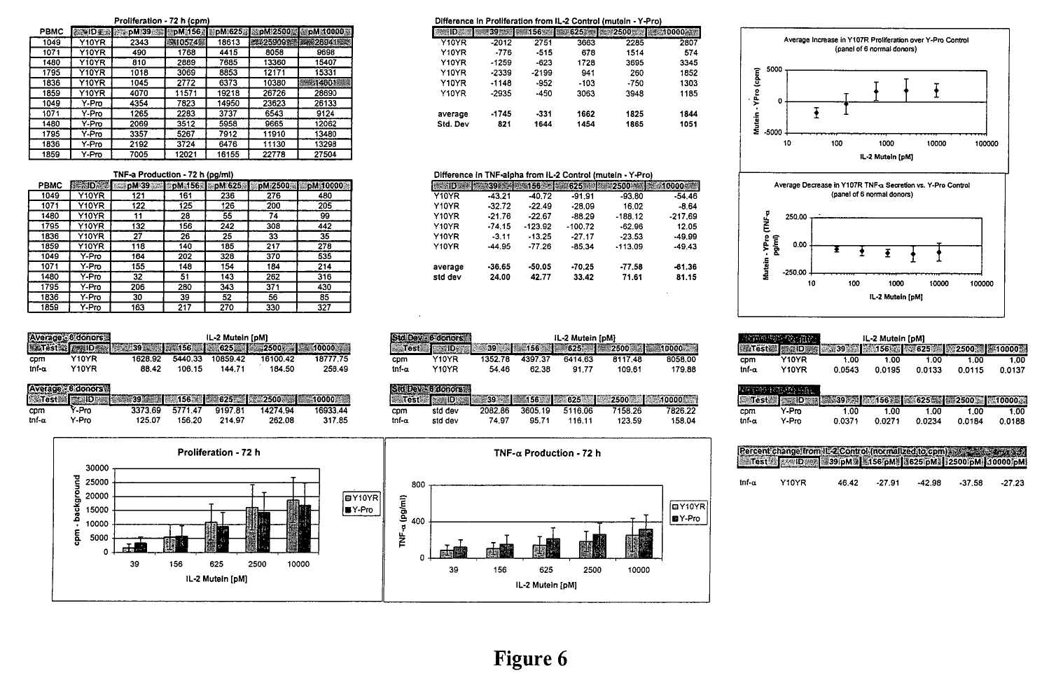

Five beneficial IL-2 mutations that reduce pro-inflammatory cytokine production while maintaining or increasing levels of proliferation and cytotoxicity in normal human PBMC were identified. For the data set presented below, IL-2 muteins were tested along with the relevant control, i.e., des-alanyl-1, C125S human IL-2 expressed and purified in the same yeast system (designated Y-Pro). Initially IL-2 muteins were tested in the combination proliferation/pro-inflammatory cytokine production assay over a dose response curve (39 pM-10 nM) in two independent assay setups, each with three normal blood donor PBMC tested in duplicate. Data analysis included individual donor profiles, mean±standard deviation, analysis of differences from internal IL-2 controls, and normalization of cytokine production (pg/ml) to proliferation (cpm) to derive relative levels of cytokine produced per cell. Finally, the percent decrease in TNF-α production from the IL-2 control was calculated. IL-2 muteins with a decrease in TNF-α production greater than 25% at 10,000 pM were deemed beneficial if levels of proliferation were maintained. Table 19 summarizes the percent decrease in TNF-α production observed for the 5 beneficial IL-2 muteins, which had the indicated additional amino acid substitution in the des-alanyl-1, C125S human IL-2 mutein backbone. FIGS. 2-6 show the proliferation and TNF-α production mediated by the F42E, L94Y, E95D, E95G, and Y107R muteins, respectively, in human PBMC.

TABLE 19
Percent decrease in TNF-α production from IL-2 control¹

| ID | 625 pM | 2500 pM | 10000 pM |
|---|---|---|---|
| Y107R | −43.0 | −37.6 | −27.2 |
| L94Y | −24.6 | −24.2 | −30.3 |
| E95D | −25.4 | −21.4 | −27.9 |
| E95G | −25.9 | −19.9 | −25.6 |
| F42E | −15.9 | −17.3 | −26.0 |

¹Values represent average percent decrease from Y-Pro control from panel of 6 normal human PBMC donors.
Cytokine data was normalized to proliferation.

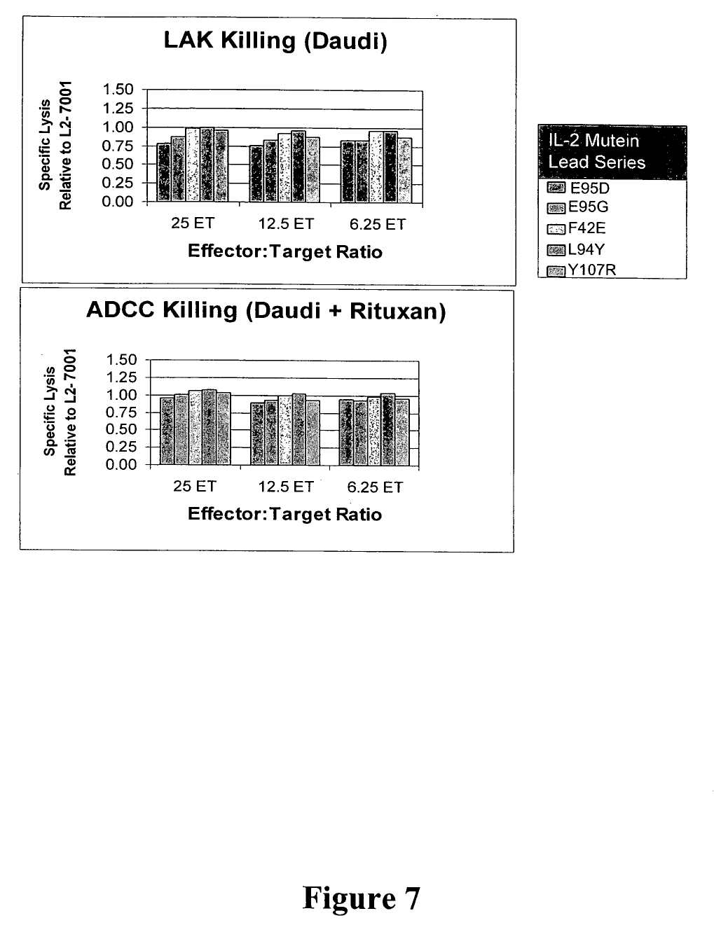

Once the 5 beneficial IL-2 muteins were identified, it was important to determine whether PBMC stimulated with IL-2 mutein retained the capacity to lyse tumor cell targets by NK, LAK, and ADCC activity. As indicated in FIG. 7, there was no difference observed between IL-2 mutein and relevant IL-2 control in the ability to lyse tumor targets by LAK and ADCC activity.

### Example 11

### Evaluation of Efficacy of IL-2 Muteins using a B 16F10 Melanoma Model

Experimental Design

The Y107R, F42E, and E95D IL-2 muteins were tested in an IL-2 sensitive B16F10 melanoma model. The objectives were to evaluate dose response, determine the minimum effective dose (MED), and demonstrate efficacy in terms of inhibition of lung metastases.

C57BL/6 mice were implanted intravenously with B16F10 melanoma cells (50,0000 cells/mouse) on day one of the study. Mice were 4-6 weeks old and randomized into groups of ten based on body weight. All groups had mean body weights within 20% of one another. Treatments were administered to mice on day two and consisted of IL-2 in the form of Proleukin®, RL-2 (research grade IL-2 from E. Coli), L2-7001® (a monomeric formulation of IL-2), or an IL-2 mutein, either E95D or Y107R. Two different dosage regimens were tested: 1) a modified “Sleijfer” regimen based on the protocol described by Sleijfer et al. (J. Clin. Oncol. 10(7): 1119-1123, 1992), consisting of two weeks of treatment with IL-2 administered subcutaneously once a day for 5 days a week (5 days on/2 days off/5 days on, with dose-up design), and 2) a regimen in which IL-2 was administered thrice weekly. The efficacy and tolerance of treatment were evaluated based on a determination of the number of lung metastases (on days 18-21, blinded), clinical observation, and measurement of body weight as an indicator of drug tolerability.

Results

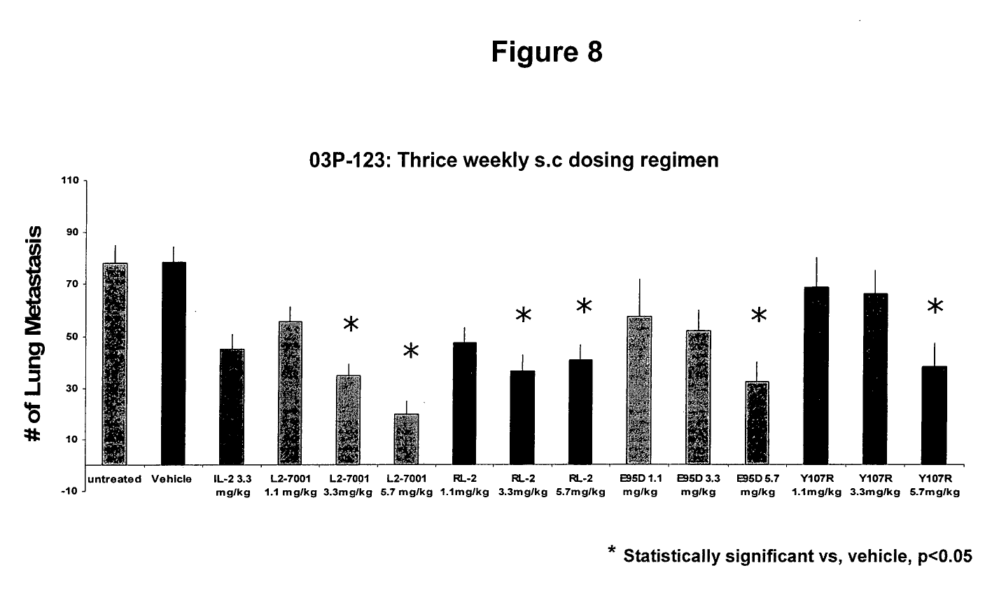

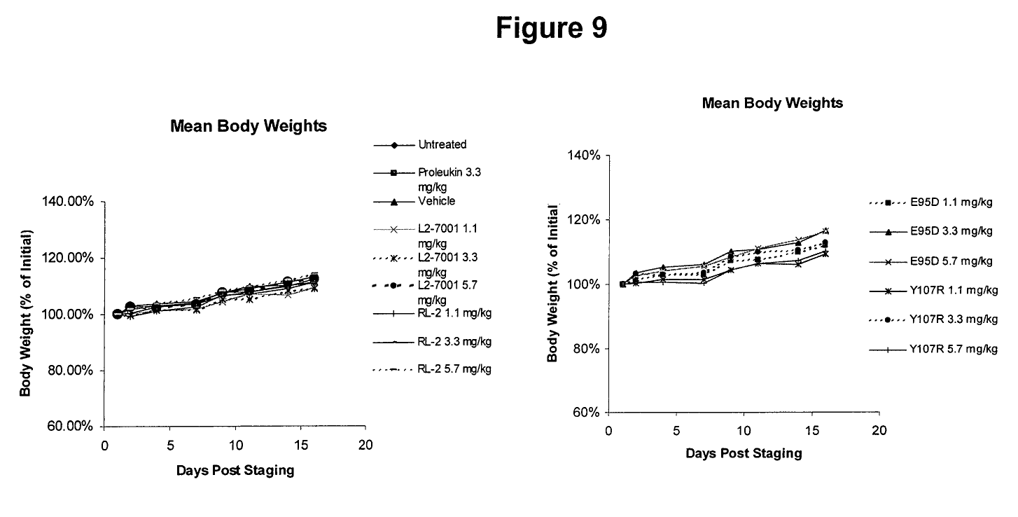

Proleukin®, RL-2, L2-7001®, and the IL-2 muteins, E95D and Y107R, were administered thrice weekly intravenously in murine B16F10 melanoma lung metastases models in C57BL/6 mice (FIGS. 8 and 9). The minimum effective dose (MED) of the IL-2 test agents (dose that was statistically significant vs. the pharmaceutical vehicle) were as follows: Proleukin® (3.3 mg/kg), L2-7001® (3.3 mg/kg), RL-2 (3.3 mg/kg), E95D (5.7 mg/kg), and Y107R (5.7 mg/kg). The ED50's (50% inhibition of metastases compared to pharmaceutical vehicle) of test agents were 2.4 mg/kg for L2-7001®, 4.8 mg/kg for E95D, and 6.1 mg/kg for Y107R. Y107R and E95D dosed at 5.7 mg/kg (3×/wk) produced equivalent inhibition of metastases compared to IL-2 benchmarks (Proleukin®, L2-7001). The maximum tolerated dose (MTD) was not reached for muteins or L2-7001®. All doses of test agents were well tolerated, and mice exhibited normal body weights throughout the study duration (FIG. 9). See Table 20 below, which provides a summary of efficacy results.

TABLE 20
Efficacy of Proleukin, RL-2, L2-7001, and IL-2 muteins E95D and
Y107R dosed thrice weekly in the murine B16F10 melanoma lung metastasis model.

| | | | | | | | P value | Total |
|---|---|---|---|---|---|---|---|---|
| | | | Number of | Metastases | % | | vs | Dose |
| Group | Mean | Std Dev | Metastases | Incidence | Inhibition | CR | Vehicle* | (mg) |
| Untreated | 78 | 27 | 24-156 | 15/15 | | | | |
| Vehicle | 79 | 19 | 38-107 | 10/10 | | | | |
| Proleukin 3.3 mg/kg | 45 | 20 | 4-68 | 10/10 | 43 | 0/10 | 0.062 | 0.36 |
| L2-7001 1.1 mg/kg | 56 | 19 | 24-88 | 10/10 | 29 | 0/10 | 0.315 | 0.12 |
| L2-7001 3.3 mg/kg | 35 | 14 | 19-64 | 10/10 | 56 | 0/10 | 0.006 | 0.36 |
| L2-7001 5.7 mg/kg | 20 | 16 | 0-38 | 7/9 | 75 | 2/9 | <0.001 | 0.62 |
| RL-2 1.1 mg/kg | 47 | 19 | 20-76 | 9/9 | 40 | 0/9 | 0.102 | 0.12 |
| RL-2 3.3 mg/kg | 37 | 18 | 8-70 | 9/9 | 53 | 0/9 | 0.014 | 0.36 |
| RL-2 5.7 mg/kg | 41 | 19 | 27-74 | 10/10 | 48 | 0/10 | 0.025 | 0.62 |
| E95D 1.1 mg/kg | 58 | 45 | 0-130 | 9/10 | 27 | 1/10 | 0.334 | 0.12 |
| E95D 3.3 mg/kg | 52 | 25 | 11-86 | 10/10 | 34 | 0/10 | 0.197 | 0.36 |
| E95D 5.7 mg/kg | 32 | 23 | 11-81 | 9/9 | 59 | 0/9 | 0.004 | 0.62 |
| Y107R 1.1 mg/kg | 69 | 37 | 16-148 | 10/10 | 13 | 0/10 | 0.646 | 0.12 |
| Y107R 3.3 mg/kg | 66 | 27 | 20-104 | 9/9 | 16 | 0/9 | 0.673 | 0.36 |
| Y107R 5.7 mg/kg | 38 | 27 | 0-84 | 8/9 | 51 | 1/9 | 0.017 | 0.62 |

*ANOVA/Student-Newmans-Keul's test

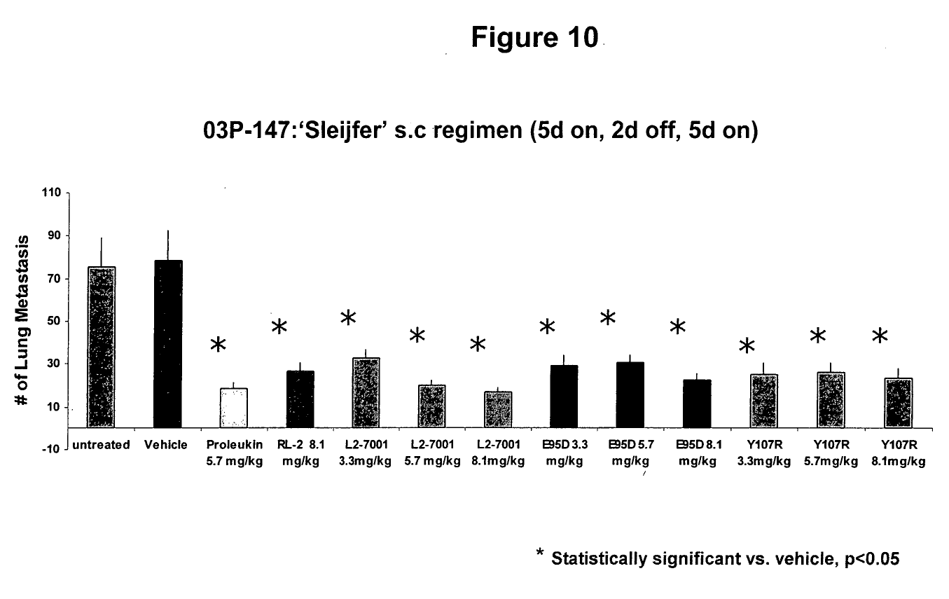

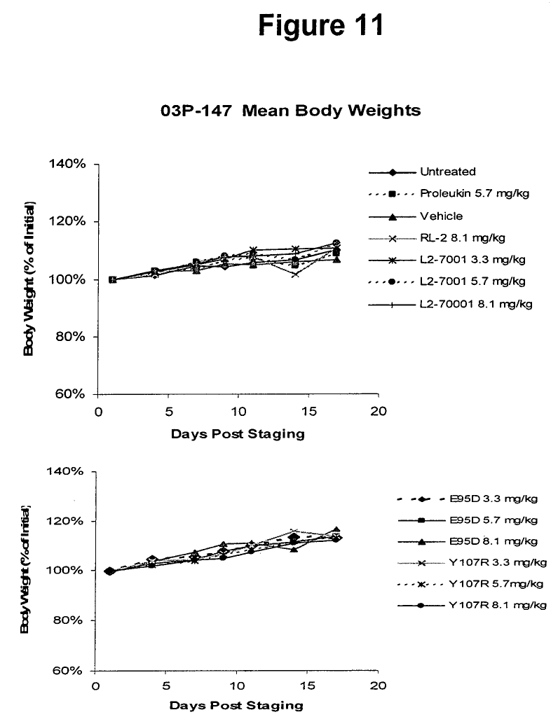

The IL-2 agents were then tested using the “Sleijfer” dosage regimen (5 days on/2 days off/5 days on). E95D and Y107R at all doses (3.3, 5.7 and 8.1 mg/kg) showed significant reduction in the mean number of lung metastases compared to vehicle-treated or untreated mice (p<0.001 ANOVA/Student-Newman-Keul's test), and efficacy was equivalent to IL-2 benchmarks (Proleukin®, L2-7001®) (FIGS. 10 and 11). Y107R at 3.3 and 8.1 mg/kg demonstrated 2/10 and 1/10 of a complete response (CR), respectively (Table 21), where a CR is defined as the complete disappearance of tumors (including microscopic lesions) in the mouse lung. Proleukin® at 5.7 mg/kg and RL-2 at 8.1 mg/kg and L2-7001® at 3.3, 5.7 and 8.1 mg/kg significantly reduced lung metastasis compared to vehicle-treated or untreated mice (p<0.001 ANOVA/Student-Newman-Keul's test). The minimum effective dose was 3.3 mg/kg for L2-7001, E95D and Y107R and the computed ED50's of E95D, Y107R and L2-7001 were <3.3 mg/kg. All doses of test agents up to 8.1 mg/kg were well tolerated, and mice exhibited normal body weights. MTD was not achieved (FIG. 11). See Table 21 below, which provides a summary of efficacy results.

TABLE 21
Efficacy of Proleukin, RL-2, L2-7001, and IL-2 muteins E95D and
Y107R dosed according to the “Sleijfer” protocol (5 days on/2 days off/5days on) in the
murine B16F10 melanoma lung metastasis model.

| | | | | | | | | Total |
|---|---|---|---|---|---|---|---|---|
| | | | Number of | Metastases | % | | | Dose |
| Group | Mean | Std Dev | Metastases | Incidence | Inhibition | CR | P value | (mg) |
| Untreated | 76 | 31 | 45-124 | 5/5 | | 0/5 | | |
| Vehicle | 78 | 46 | 34-170 | 10/10 | | 0/10 | | |
| Proleukin 5.7 mg/kg | 19 | 9 | 9-38 | 10/10 | 76 | 0/10 | <0.001 | 1.03 |
| RL-2 8.1 mg/kg | 27 | 14 | 12-56 | 10/10 | 66 | 0/10 | <0.001 | 1.46 |
| L2-7001 3.3 mg/kg | 33 | 13 | 17-56 | 10/10 | 58 | 0/10 | <0.001 | 0.59 |
| L2-7001 5.7 mg/kg | 20 | 7 | 12-30 | 10/10 | 74 | 0/10 | <0.001 | 1.03 |
| L2-7001 8.1 mg/kg | 17 | 6 | 12-29 | 10/10 | 78 | 0/10 | <0.001 | 1.46 |
| E95D 3.3 mg/kg | 29 | 15 | 10-47 | 10/10 | 63 | 0/10 | <0.001 | 0.59 |
| E95D 5.7 mg/kg | 31 | 11 | 16-53 | 10/10 | 61 | 0/10 | <0.001 | 1.03 |
| E95D 8.1 mg/kg | 23 | 9 | 12-32 | 10/10 | 71 | 0/10 | <0.001 | 1.46 |
| Y107R 3.3 mg/kg | 25 | 18 | 0-60 | 8/10 | 68 | 2/10 | <0.001 | 0.59 |
| Y107R 5.7 mg/kg | 26 | 15 | 11-51 | 10/10 | 66 | 0/10 | <0.001 | 1.03 |
| Y107R 8.1 mg/kg | 24 | 14 | 0-36 | 9/10 | 70 | 1/10 | <0.001 | 1.46 |

ANOVA/Student-Newmans-Keul's test

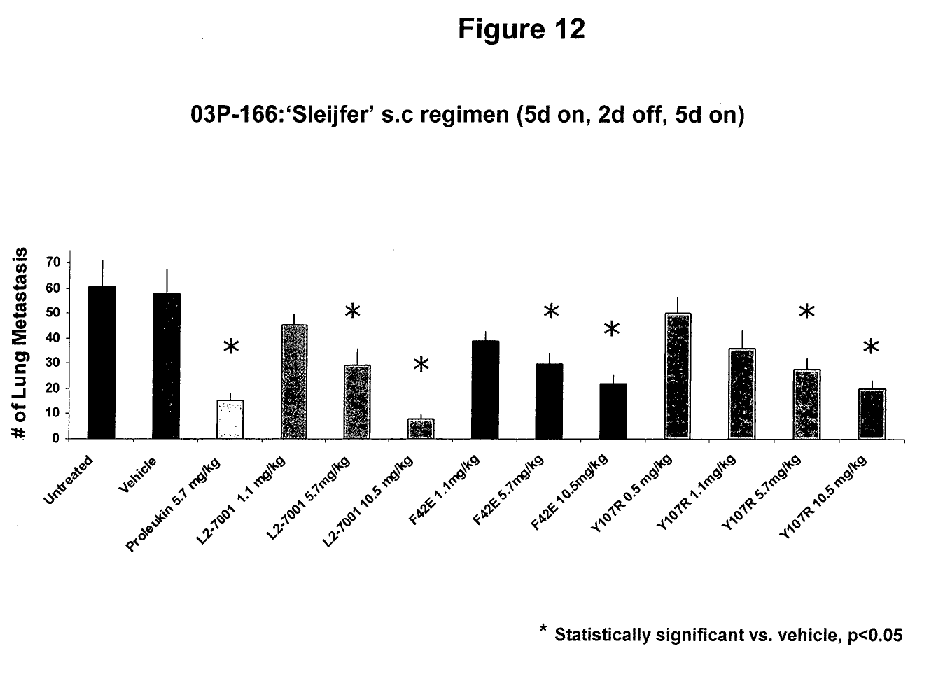

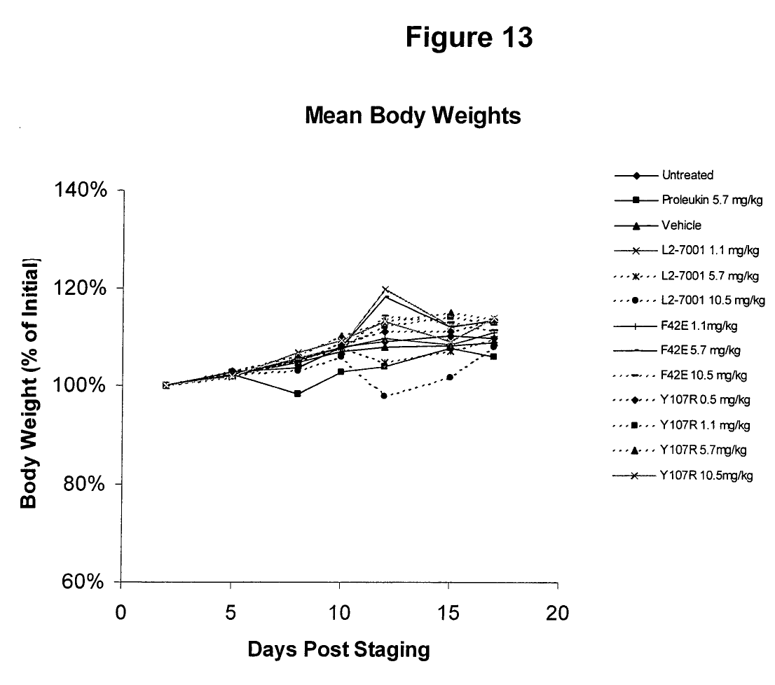

In a repeat study, the efficacies of Proleukin®, L2-7001®, Y107R, and in addition F42E were evaluated using the “Sleijfer” dosage regimen (5 days on/2 days off/5 days on) in the B16F10 melanoma lung metastasis model. Y107R, F42E, L2-7001 at 5.7 mg/kg and 10.5 mg/kg and Proleukin® at 5.7 mg/kg demonstrated a significant reduction in the number of lung metastases compared to vehicle-treated or untreated mice (p<0.05 ANOVA/Student-Newman-Keul's test) (FIGS. 12 and 13). The minimum effective dose was 5.7 mg/kg for L2-7001, F42E and Y107R, and the computed ED50's were 6.47 mg/kg for F42E, 6.33 mg/kg for Y107R, and 5.37 mg/kg for L2-7001. At similar doses, there was no difference in efficacy between test agents (Proleukin®, L2-7001®, F42E and Y107R), indicating that the IL-2 muteins demonstrated equivalent activity compared to benchmarks, Proleukin® and L2-7001®. Doses of 5.7 mg/kg Proleukin® and 10.5 mg/kg L2-7001® exhibited mouse body weight loss and were identified as MTD doses of each agent, respectively (FIG. 13). No body weight loss was observed for muteins Y107R and F42E at all doses tested (i.e., IL-2 mutein MTD were not achieved), indicating that the muteins were better tolerated that IL-2 benchmarks (FIG. 13). See Table 22 below, which provides a summary of efficacy results.

TABLE 22
Efficacy of Proleukin, RL-2, L2-7001, and IL-2 muteins E95D and
Y107R dosed according to the “Sleijfer” protocol (5 days on/2 days off/5days on) in the
murine B16F10 melanoma lung metastasis model.

| | | | | | | | | Total |
|---|---|---|---|---|---|---|---|---|
| | | | Number of | Metastases | % | | | Dose |
| Group | Mean | Std Dev | Metastases | Incidence | Inhibition | CR | P value | (mg) |
| Untreated | 61 | 35 | 28-155 | 11/11 | | 0/11 | | |
| Vehicle | 58 | 31 | 16-105 | 10/10 | | 0/10 | | |
| Proleukin 5.7 mg/kg | 15 | 7 | 6-27 | 6/6 | 74 | 0/6 | <0.001 | 1.03 |
| L2-7001 1.1 mg/kg | 45 | 15 | 26-77 | 10/10 | 22 | 0/10 | 0.294 | 0.2 |
| L2-7001 5.7 mg/kg | 29 | 22 | 0-80 | 8/10 | 50 | 2/10 | 0.019 | 1.03 |
| L2-7001 10.5 mg/kg | 8 | 5 | 0-15 | 8/10 | 87 | 2/10 | <0.001 | 1.89 |
| F42E 1.1 mg/kg | 39 | 12 | 20-55 | 10/10 | 33 | 0/10 | 0.131 | 0.2 |
| F42E 5.7 mg/kg | 30 | 14 | 15-61 | 9/9 | 48 | 0/9 | 0.025 | 1.03 |
| F42E 10.5 mg/kg | 22 | 10 | 10-33 | 9/9 | 62 | 0/9 | 0.003 | 1.89 |
| Y107R 0.5 mg/kg | 50 | 19 | 25-80 | 10/10 | 13 | 0/10 | 0.358 | 0.1 |
| Y107R 1.1 mg/kg | 36 | 22 | 10-72 | 9/9 | 38 | 0/9 | 0.105 | 0.2 |
| Y107R 5.7 mg/kg | 28 | 13 | 9-48 | 9/9 | 52 | 0/9 | 0.022 | 1.03 |
| Y107R 10.5 mg/kg | 20 | 10 | 10-39 | 10/10 | 65 | 0/10 | 0.001 | 1.89 |

ANOVA/Student-Newmans-Keul's test

Conclusions

1. E95D, Y107R and F42E IL-2 muteins retain antitumor activity in vivo.

2. The efficacy of the E95D, Y107R, and F42E IL-2 muteins in the classical B16 melanoma metastases model is equivalent to benchmarks Proleukin® or L2-7001® at similar doses when administered thrice weekly or according to the “Sleijfer” regimen.

3. The Y107R and F42E IL-2 muteins are better tolerated than IL-2 benchmarks Proleukin® and L2-7001®, retain IL-2 activity, and demonstrate a superior therapeutic index.

4. Higher maximum tolerated doses (MTD) can be achieved with the Y107R and F42E IL-2 muteins and may allow higher dose intensification in clinical regimens, which could translate into improved efficacy.

### Example 12

### Antitumor Activity of Muteins in Xenograft Models of Non-Hodgkin's Lymphoma

Experimental Design

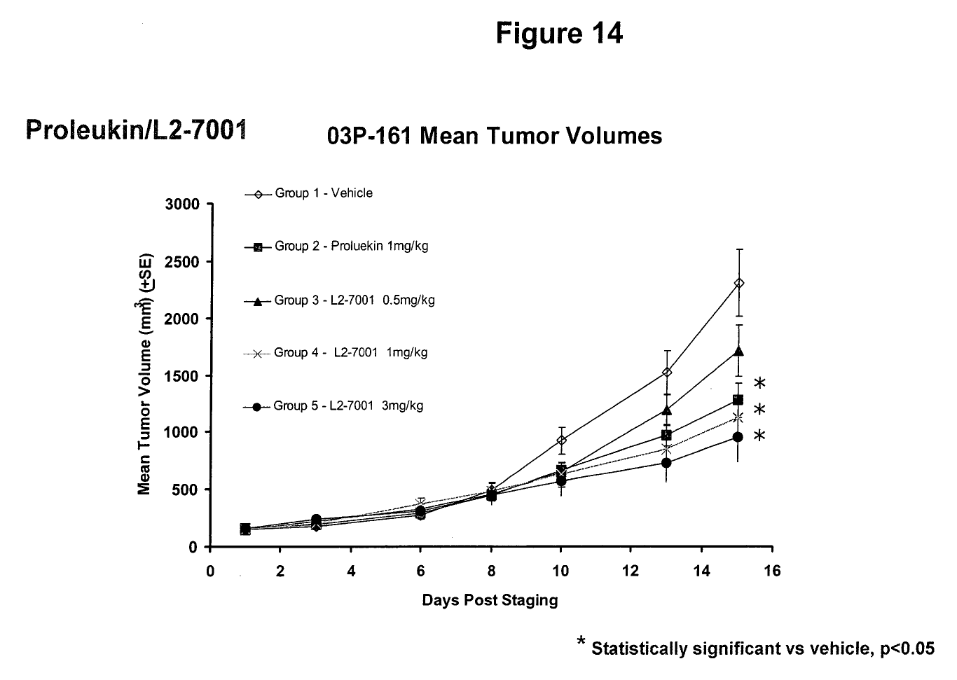

The objectives of these studies were to evaluate the activity of Proleukin® (IL-2), L2-7001®, E95D, and Y107R as single agents or in combination with the monoclonal antibody rituximab (Rituxan®; IDEC-C2B8; IDEC Pharmaceuticals Corp., San Diego, Calif.) in NK (or immune effector cell)/ADCC-mediated efficacy models (FIGS. 14-20). Efficacy of IL-2 muteins was evaluated in two distinct non-Hodgkin's lymphoma (NHL) models, i.e., Namalwa (high grade NHL) model which is sensitive to IL-2 and the Daudi model (low grade NHL, CD20+), which displays marginal activity with IL-2; but is responsive to rituximab.

Results

Athymic nude BALB/c mice were acclimated for 1 week prior to inoculation with either Namalwa or Daudi cells. Namalwa or Daudi cells (5×10⁶ cells/mouse) were implanted subcutaneously in the right flank of irradiated young nude mice (3Gy˜3.2 mins) with 50% matrigel at a volume of 0.1 mL. Treatment began when the average tumor volume was 100-200 mm³. This was designated as day 1 of the study. Tumor volumes and body weight measurements were evaluated twice a week. Clinical observations were noted. Individual mice with tumor volumes greater than 3000 mm³ or groups with mean tumor volumes greater than 2000 mm³ were euthanized. Mice with body weight loss greater than 20% were also sacrificed. Endpoints were tumor volume measurements, body weights and clinical observations.

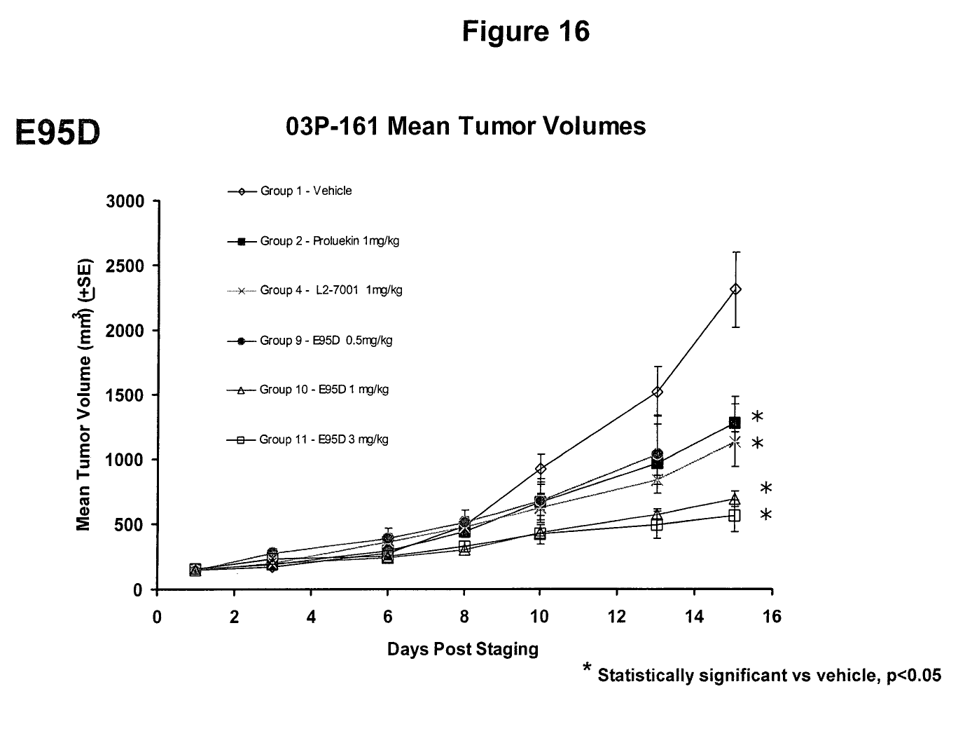

The efficacies of thrice weekly regimens of Proleukin®, L2-7001®, muteins Y107R or E95D were evaluated in a staged, aggressive human NHL model (Namalwa) in irradiated Balb/c nude mice (FIGS. 14-16). Single agent L2-7001® showed a good dose-response effect with a calculated ED50 of 2 mg/kg (FIG. 16). Compared to treatment with vehicle alone, the activity of L2-7001® was significantly different at 1 mg/kg, 3 times per week (p=0.038) and 3 mg/kg, 3 times per week (p=0.009). However, there was no statistical difference between treatment with L2-7001 1 at mg/kg, 3 times per week versus treatment with Proleukin® at 1 mg/kg, 3 times per week (p>0.05) (FIG. 16?).

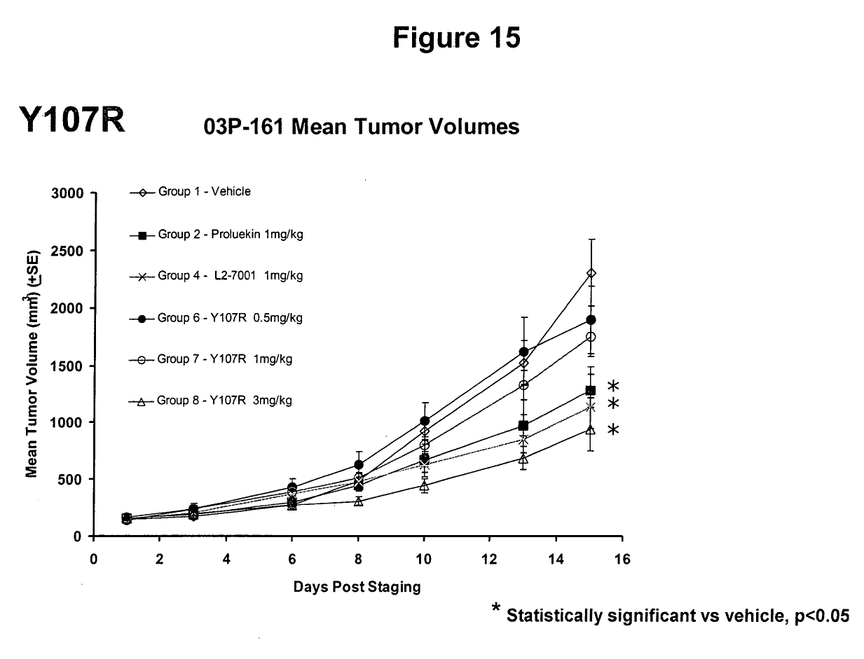

The IL-2 muteins Y107R and E95D demonstrated a dose response effect in the Namalwa tumor model (FIGS. 15 and 16). Treatment with E95D at 1 and 3 mg/kg, 3 times per week was significantly different vs. treatment with vehicle (p<0.001), whereas, the minimum effective dose of Y107R was slightly higher (3 mg/kg) in this model. Treatment with Y107R and E95D at 1 mg/kg, 3 times per week demonstrates equivalent activity to benchmarks Proleukin® and L2-7001® at 1 mg/kg, 3 times per week (p>0.05) in the Namalwa model. The muteins were tested up to 3 mg/kg and the MTDs were not reached.

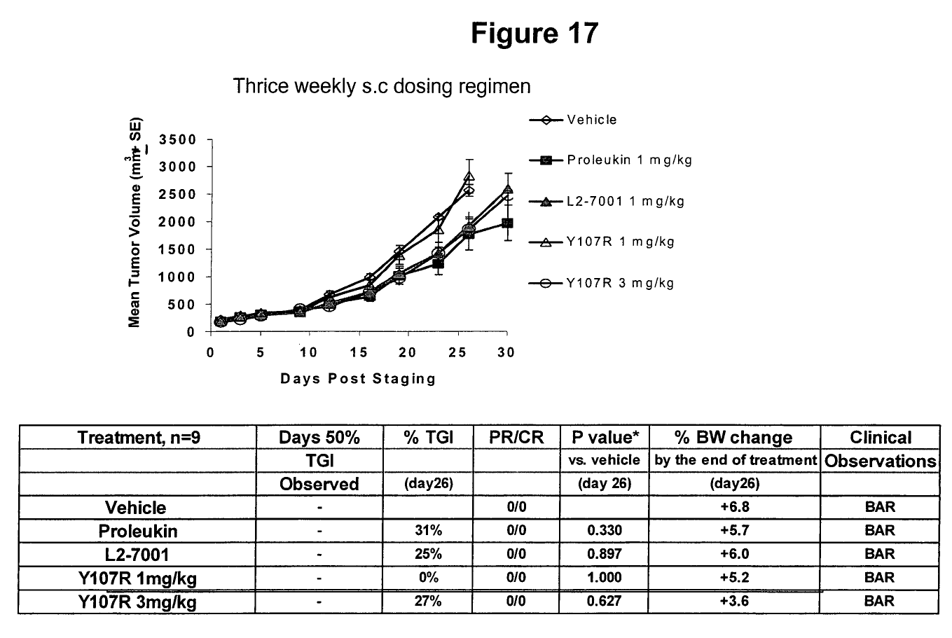

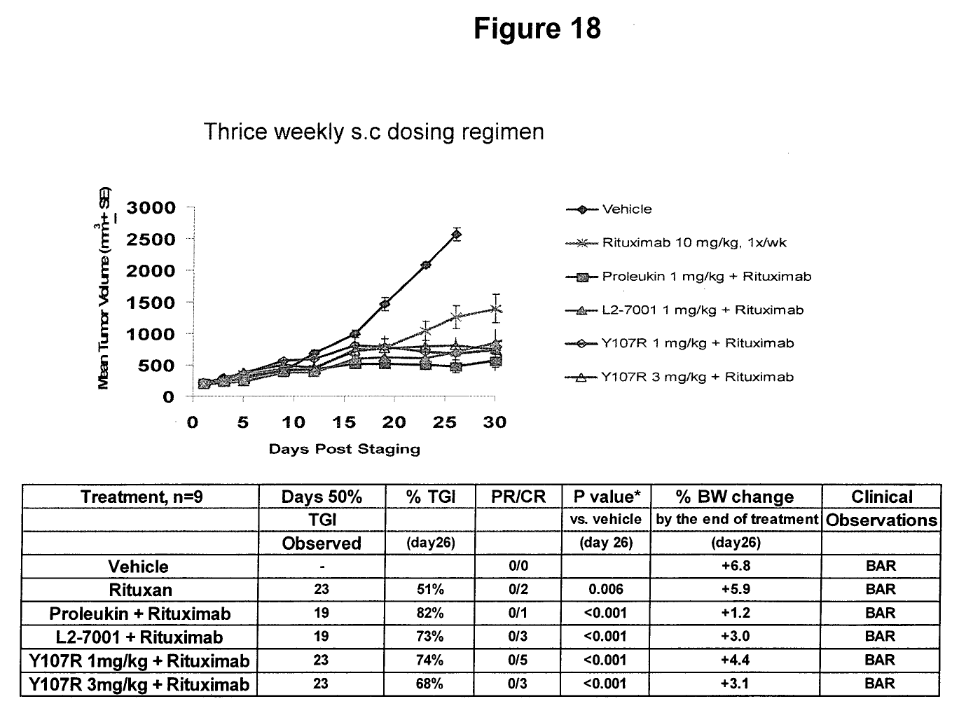

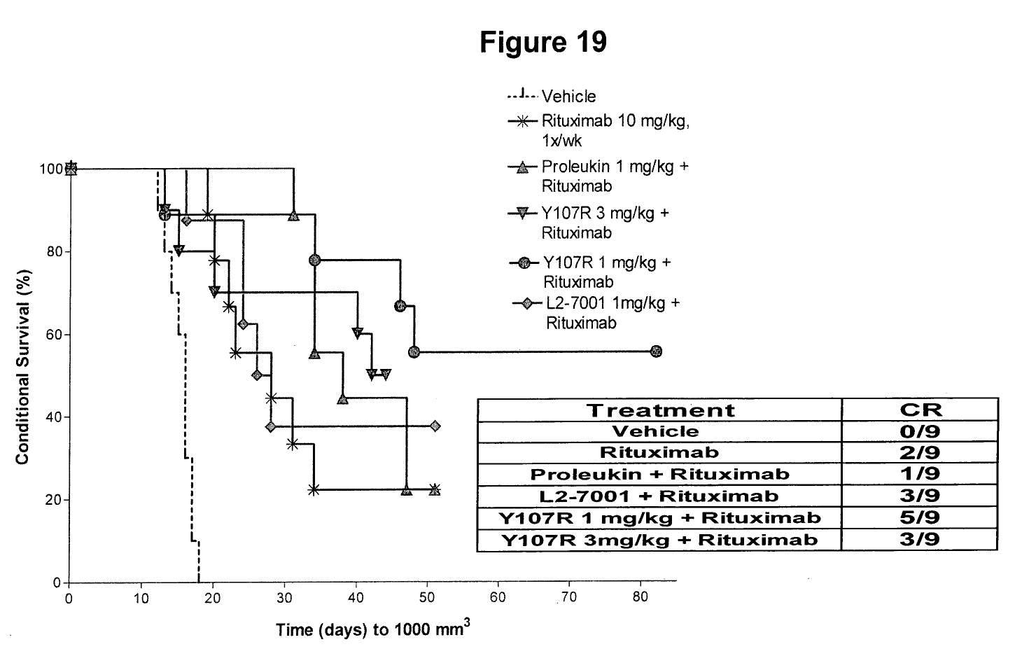

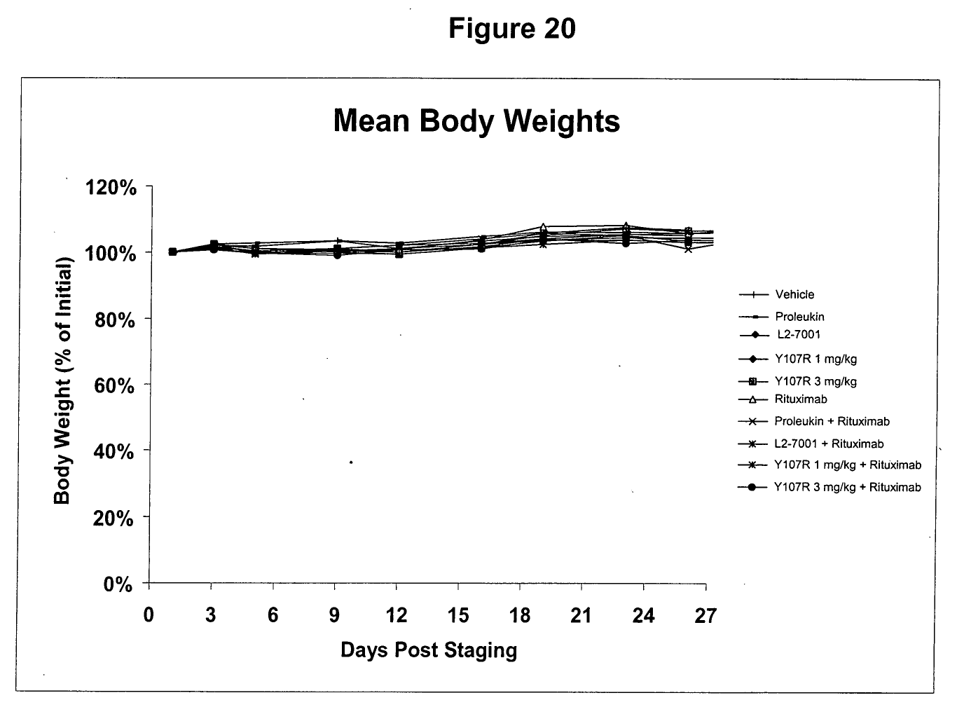

The efficacies of combination therapy with thrice weekly regimens of Proleukin®, L2-7001®, or the IL-2 mutein Y107R with rituximab in a CD20+ low grade human NHL Daudi xenografts in Balb/c nude mice were evaluated (FIGS. 17-20). The objective of these experiments was to evaluate the role of in vivo activation of effector cells (NK, monocytes, macrophages, neutrophils) on the efficacy of combination therapy with IL-2 and therapeutic antibodies (rituximab). Inhibition of tumor growth by treatment with single agents, Proleukin®, L2-7001®, or Y107R, at 3 mg/kg is not statistically different from treatment with vehicle (p>0.05, ANOVA day 26) (FIG. 17). However, when analyzed based on tumor growth delay (i.e, days for tumor progression to 1000 mm³), statistical differences were observed compared to vehicle treatment (p<0.05, Lon Rank test). Significant augmentation of tumor efficacy was observed for treatment with Proleukin® or L2-7001® in combination with rituximab versus respective single agents (p<0.05, ANOVA day 26) (FIGS. 18 and 19). The Y107R mutein induces similar augmentation of tumor efficacy as Proleukin/L2-7001 when administered in combination with rituximab in the Daudi human xenograft model of B-cell lymphoma. Treatment with the combination of Y107R and rituximab resulted in an increased number of durable responses (5 CR) and improved conditional survival compared to treatment with Proleukin® (FIGS. 18 and 19). All doses of single agent IL-2 muteins and combinations with rituximab were well tolerated (FIG. 20).

Conclusions

1. Muteins E95D and Y 107R demonstrate significant inhibition of tumor growth of aggressive B-cell NHL (Namalwa model of NHL).

2. IL-2 muteins may be effective as a single agent in subset cancer populations, including melanoma, NHL.

3. Activity of the IL-2 mutein Y107R is marginal against low grade B-cell NHL Daudi model, but is capable of activating immune effector cells (i.e., NK, monocytes, macrophages, neutrophils) to potently mediate ADCC when combined with rituximab

4. The IL-2 mutein Y107R and rituximab in combination therapy demonstrate superior efficacy compared to single agents IL-2 or rituximab alone.

5. Activity of IL-2 muteins could be applicable to combinations with other antibodies that mediate effects through ADCC or similar immune cell effector mechanims.

6. The implications of these findings could be applicable to other cytokines molecules with mechanistic effects similar to IL-2 (or muteins) to mediate effector cell responses that could be applicable to other therapies or disease indications

### Example 13

### Evaluation of Tolerability in IL-2-Induced Vascular Leak Syndrome Model

Experimental Design

Female C57BL/6 mice were acclimated for 1 week prior to the start of the study. Mice were 8-10 weeks old and randomized into groups of five based on body weight. Proleukin®, L2-701(, or an IL-2 mutein, E95D or Y107R, was injected intraperitoneally (i.p.) at 6 mg/kg (˜2,000,000 IU), 3 times per day. Injections were repeated for 10 doses. ¹²⁵I-albumin (1 μCi, PerkinElmer Life Sciences Inc. Boston, Mass.) in 0.1 ml PBS containing 1% mouse serum was injected 4 hours after the Proleukin® dose on day 4. Mice were euthanized 60 minutes after the injection with ¹²⁵I-albumin. The lungs were harvested and placed in a vial for gamma counting.

Results

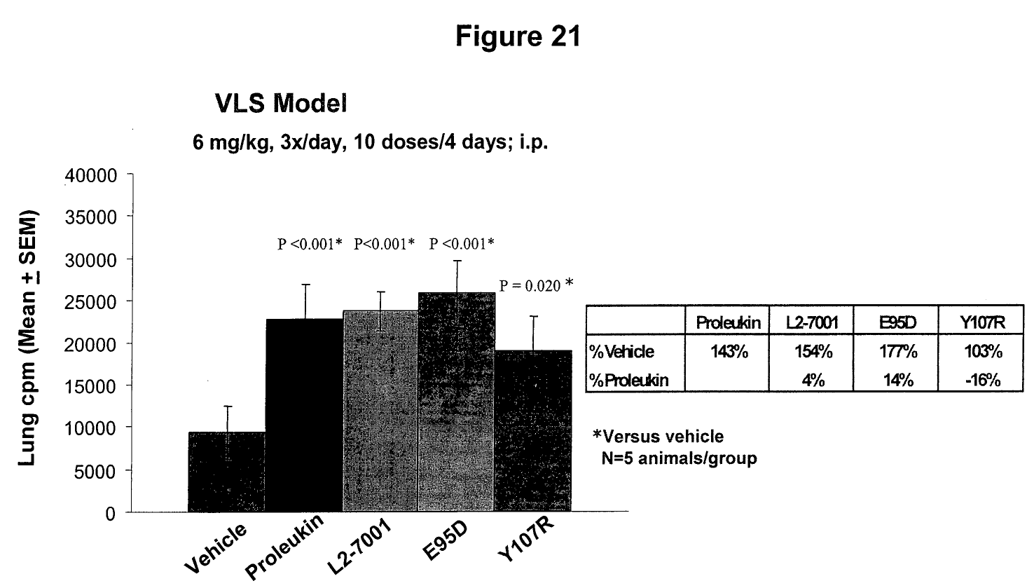

Treatment with high doses of IL-2 or L2-7001® (6 mg/kg, i.p., 3×/day, 10 doses) produced a statistical increase in ¹²⁵I-albumin retention in the lungs of mice, resulting from increased vascular leak and mimicking a pathological model of vascular leak syndrome (VLS) similar to that seen in humans (FIG. 21). In this “acute” model of experimental VLS, both the E95D and Y107R IL-2 muteins caused increases in ¹²⁵I -albumin retention; however, Y107R demonstrated a 16% reduction in the extent of VLS induction compared to treatment with Proleukin® (FIG. 21).

### Example 14

### Evaluation of Tolerability of IL-2 Muteins by Monitoring Body Temperature Changes Using a Temperature Chip

Introduction

Although, the precise mechanism underlying IL-2 induced toxicity and VLS is unclear, accumulating data suggests that IL-2 induced natural killer cells (NK) trigger dose-limiting toxicities as a consequence of overproduction of pro-inflammatory cytokines including IFN-γ, TNF-α, TNF-β, IL-1β, and IL-6 that activate monocytes/macrophages and induce nitric oxide (NO) production leading to subsequent damage of endothelial cells (Dubinett et al. (1994) Cell Immunol. 157(1):170-180; and Samlowski et al. (1995) J. Immunother. Emphasis Tumor Immunol. 18(3):166-78).

Fever and chills are common Grade 3 adverse events during IL-2 therapy. Fever is a physiological reaction to TNF-α inducing prostaglandin E2 and the onset of fever induces vasoconstriction and shivering preceding the actual change in core temperature. IL-2 does not directly induce prostaglandin E2 synthesis in vitro; therefore IL-2 itself is classified as a non-pyrogenic cytokine. However, following exogenous administration, IL-2 induces the release of pyrogenic cytokines, particularly TNF-α, a major cause of fever and other aspects of acute phase response during IL-2 therapy (Mier, et al. (1988) J. Clin. Immunol. 8:426). It has been reported that plasma levels of TNF-α can reach greater than 600 ng/ml in patients treated with IL-2 (Gemlo et al. (1988) Cancer Res. 48(20):5864-5867).

Dose-limiting toxicities in humans (e.g., fever/chills, VLS, and hypotension) all have derivative correlations with pro-inflammatory cytokine and NO production. Since there is a direct relationship between production of pro-inflammatory cytokines, such as TNF-α, and the induction of changes in physiological body temperature, temperature changes can be monitored as a predictor of tolerability following IL-2 immunotherapy.

To model the profile of adverse events, it is important to extrapolate the relationship between temperature changes and pro-inflammatory cytokine production from human to mouse. In both species, production of pro-inflammatory cytokines (TNF-α, IL-1β, etc.) is a cause of body temperature changes, mediated by hypothalamic induction of prostaglandin E2. Commonly used experimental mammals, such as the mouse exhibit hypothermia and hypometabolism when exposed acutely to many drugs. This is postulated as an inherent, protective response to reduce lethality of toxic insult (Gordon and Yange (1997) Ann. N.Y. Acad. Sci. 813:835).

While one might predict an increase in core body temperature following IL-2 administration in a mouse model, a decrease is actually observed in this model. It is known in the art that exogenous mediators of inflammation, such as LPS induce TNF-α and temporally decrease core body temperature in a mouse model (Kozak et al. (1997) Ann. NY Acad. Sci. 813:835). In another model, telemetric evaluation of hypothermic body temperature proved to be an early, significant indicator of mortality in a murine model of SEB enteric shock (Vlach et al. (2000) Comp. Med. 50:160).

Experimental Design

C57BL/6 mice were implanted subcutaneously with a temperature chip, and a POCKET SCANNER system (BioMedic Data Systems, Inc. Seaford, Del.) was used to monitor changes in body temperature. Proleukin®, L2-701®, or an IL-2 mutein, E95D, Y107R, L94Y, or F42E were administered subcutaneously using the “Sleijfer” dosage regimen (5 days on/2 days off/5 days on). The body temperatures of mice were monitored at given time points before and after administration of IL-2 and compared to mice injected with a buffer control (vehicle). Endpoints were core body temperature, clinical observations, body weight, and plasma pro-inflammatory cytokine (e.g., TNF-α) levels.

Results

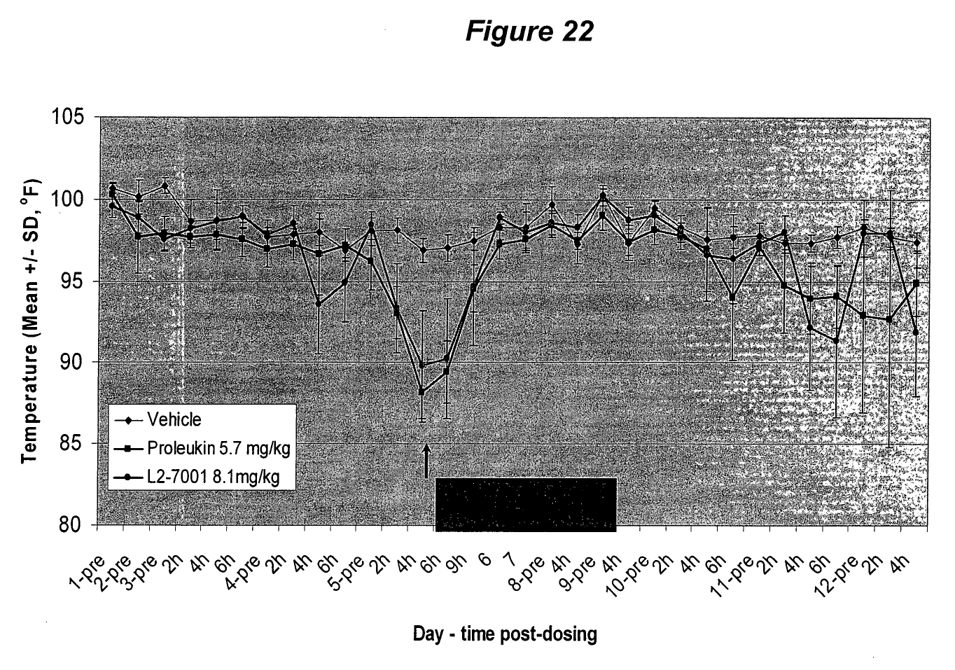

Following Proleukin® or L2-7001® administration (5.7 mg/kg or 8.1 mg/kg, daily subcutaneous injection for 5 days), significant decreases in temperature were observed 4 hours post dosing on days 4 and 5. Although, there was a decrease in temperature (not an increase as observed in humans), the effect was reproducible, and no effect was observed with vehicle-treated animals. FIG. 22 depicts an expanded time course performed to include temperature monitoring up to 9 hours post-dosing for 10 doses over a 2-week period. The most consistent, significant changes occurred at 4 hours post dosing on day 5. Furthermore, there was a significant correlation between IL-2 induced temperature changes and plasma TNF-α levels in the mouse model. The model is reproducible, and results in significant decreases in temperature in response to IL-2 treatment as summarized in Table 23.

TABLE 23
Summary of Temperature Changes at 4 hours Post-Dosing.
Week 1

| | | | Proleukin | L2-7001 | L2-7001 |
|---|---|---|---|---|---|
| Study ID | n | Vehicle¹ | (5.7 mpk) | (5.7 mpk) | (8.1 mpk) |
| 03P-122 | 5 | 97.6 ± 0.9 | 90.7 ± 2.9 (P < 0.01) | 91.2 ± 2.2 (P < 0.01) | 87.6 ± 1.0(P < 0.01) |
| 03P-145² | 5 | 97.0 ± 0.7 | 95.4 ± 1.5 (P = 0.05) | 97.3 ± 1.3 (NS) | ND |
| 03P-147³ | 5 | 98.3 ± 0.7 | 93.7 ± 1.6 (P < 0.05) | 94.6 ± 3.9 (NS) | ND |
| 03P-159 | 10/5 | 98.1 ± 0.4 | 89.4 ± 2.1 (P < 0.001) | 95.4 ± 3.6(P = 0.05) | 93.1 ± 2.7(P < 0.001) |
| Tox03-002 | 10 | 96.9 ± 0.7 | 88.2 ± 1.6 (P < 0.001) | 91.7 ± 4.9 (P < 0.001) | 89.8 ± 3.4 (P < 0.001) |

Week 2

| | | | Proleukin | L2-7001 | L2-7001 |
|---|---|---|---|---|---|
| Study ID | n | Vehicle | (5.7 mpk) | (5.7 mpk) | (8.1 mpk) |
| 03P-122 | 5 | 98.0 ± 0.7 | 91.9 ± 5.4 (P < 0.01) | 87.8 ± 1.9 (P < 0.01) | 86.9 ± 1.1 (P < 0.01)* |
| 03P-145² | 5 | 96.2 ± 1.2 | 93.4 ± 2.4 (P = 0.033) | 97 ± 1.4 (NS) | 97.2 ± 0.6 (NS) |
| 03P-147³ | 5 | 98.4 ± 1 | 95.7 ± 2.1 (P < 0.05) | 91.9 ± 3.7 (NS) | ND |
| 03P-159 | 10/5 | 97.1 ± 0.6 | 87.3 ± 4 (P = 0.3) | 95.3 ± 2.0 (NS) | 89.4 ± 3.6 (P = 0.03) |
| Tox03-002 | 10 | 97.4 ± 0.6 | 94.9 ± 2.0 (P < 0.05)⁴ | 94.7 ± 2.6 (NS) | 91.9 ± 3.9 (NS) |

¹Values expressed as Mean body temperature (° F.) +/− SD, statistical test (ANOVA + Studen-Newman-Keuls), not significant for p > 0.05
*Animals in group dosed at 10.5 mpk for week 2
²BALB/c mice used in study
³Animals in study tumor bearing, all groups dosed at 5.7 mpk for efficacy study
⁴One mice died 30 min after last injection due to severe hypothermia
⁵NS = Not significant (P > 0.05)

Of note, the L2-7001 formulation is better tolerated in the mouse model, as significant temperature drops are consistently observed at doses equal or greater than 8.1 mg/kg, whereas 5.7 mg/kg of Proleukin® is the maximum tolerated dose in this model. These observations are consistent with other preclinical models, which dosed for prolonged periods of time (generally 2 week dosing cycles) in tumor-bearing animals.

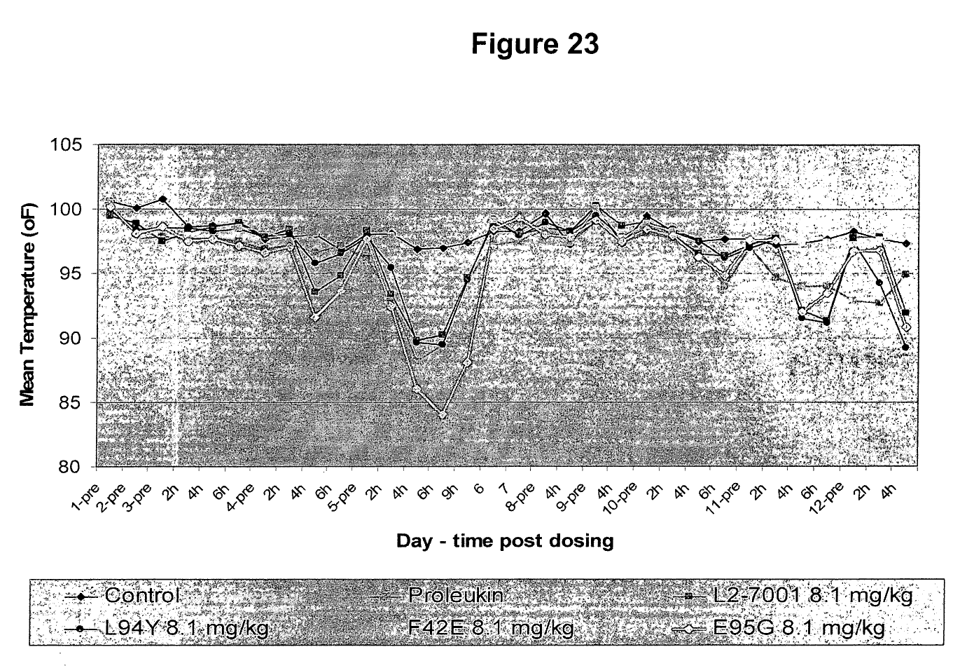

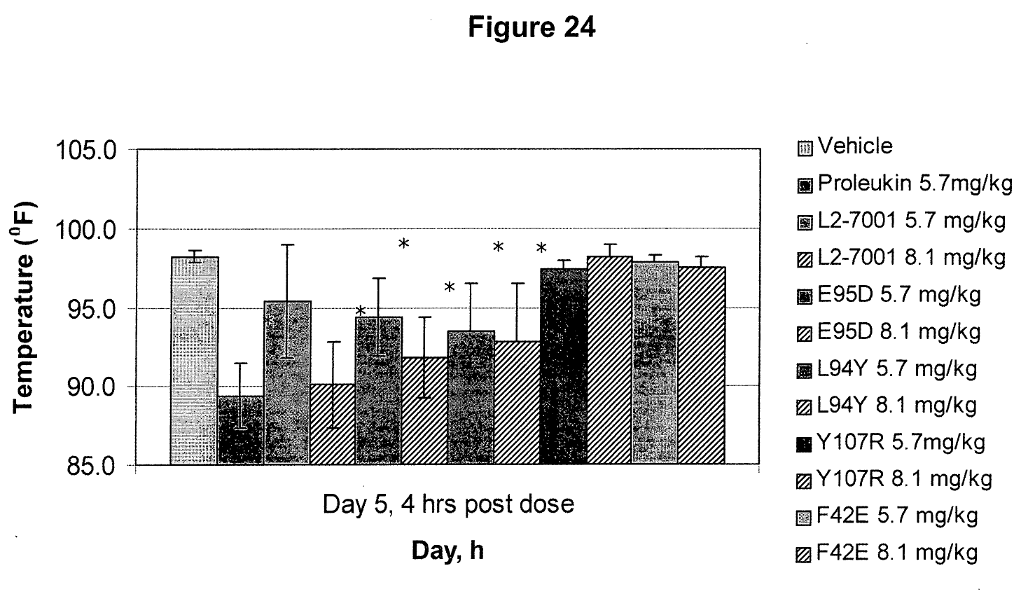

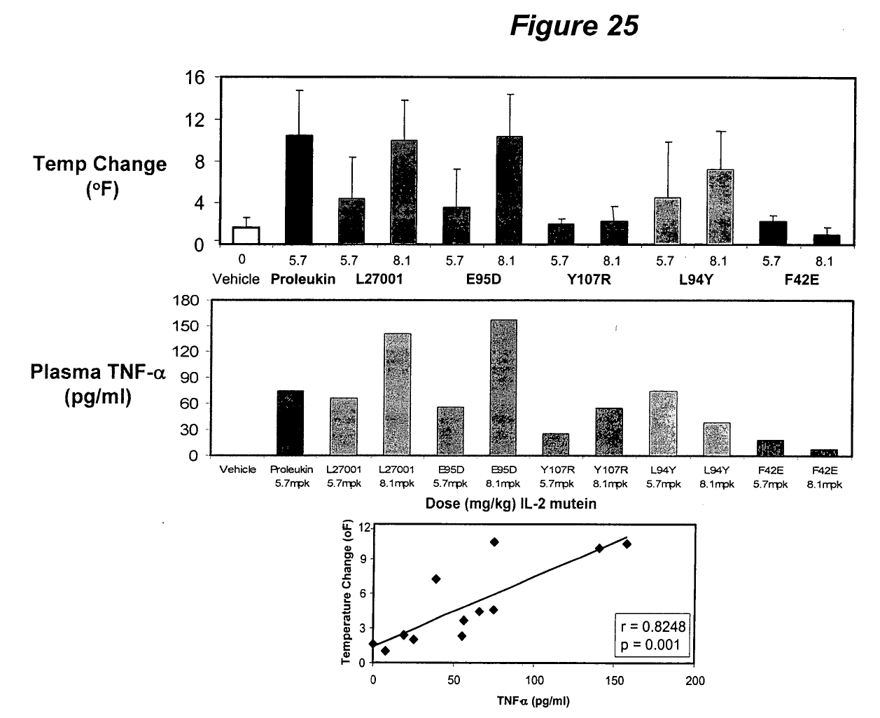

Two of the IL-2 muteins, Y107R and F42E showed significantly reduced temperature changes correlating with reduced induction of TNF-α, predictive of improved tolerability compared to Proleukin® and L2-7001®. See FIGS. 23-25.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the embodiments disclosed herein. Although specific terms are employed herein, they are used in a generic and descriptive sense only and not for purposes of limitation.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

That which is claimed:

1. An isolated nucleic acid molecule comprising a nucleotide sequence selected from the group consisting of:
a) a nucleotide sequence encoding a mutein of human IL-2, said mutein comprising an amino acid sequence selected from the group consisting of SEQ ID NOS:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, and 344;
b) the nucleotide sequence set forth in SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, or 343;
c) a nucleotide sequence encoding a mutein of human IL-2, said mutein comprising an amino acid sequence comprising residues 2-133 of the sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;
d) a nucleotide sequence comprising nucleotides 4-399 of the sequence set forth in SEQ ID NO: 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, or 343;
e) the nucleotide sequence of any one of a), b), c), or d), wherein said sequence comprises a substitution of nucleotides 373-375 of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, or 343 with a triplet codon that encodes alanine;
f) the nucleotide sequence of any one of a), b), c), or d), wherein said sequence comprises a substitution of nucleotides 373-375 of SEQ ID NO:9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 375, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, or 343 with a triplet codon that encodes cysteine; and
g) a nucleotide sequence of a), b), c), d), e), or f), wherein one or more codons encoding said mutein has been optimized for expression in a host cell of interest.

2. The isolated nucleic acid molecule of claim 1, wherein the nucleotide sequence of g) is selected from the group consisting of the sequence set forth in SEQ ID NO:345, nucleotides 4-399 of SEQ ID NO:345, the sequence set forth in SEQ ID NO:346, and nucleotides 4-399 of SEQ ID NO:346.

3. An expression vector comprising the nucleic acid molecule of claim 1.

4. A host cell comprising the nucleic acid molecule of claim 1.

5. An isolated polypeptide comprising an amino acid sequence selected from the group consisting of:
a) the amino acid sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;
b) an amino acid sequence comprising residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;
c) the amino acid sequence of a) or b), wherein said sequence comprises an alanine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344; and
d) the amino acid sequence of a) or b), wherein said sequence comprises a cysteine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

6. An isolated polypeptide comprising a mutein of human IL-2, wherein said mutein comprises the amino acid sequence set forth in SEQ ID NO:4 with a serine substituted for cysteine at position 125 of SEQ ID NO:4 and at least one additional amino acid substitution within SEQ ID NO:4, wherein said mutein: 1) maintains or enhances proliferation of natural killer (NK) cells, and 2) induces a decreased level of pro-inflammatory cytokine production by NK cells; as compared with a similar amount of des-alanyl-1, C125S human IL-2 or C125S human IL-2 under comparable assay conditions, wherein proliferation of said NK cells and pro-inflammatory cytokine production by said NK cells are assayed using the NK-92 bioassay.

7. The isolated polypeptide of claim 6, wherein said mutein further comprises a deletion of alanine at position I of SEQ ID NO:4.

8. The isolated polypeptide of claim 6, wherein said additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, 124L, K32A, K32W, N33E, P34E, P34R, P34S, P34T, P34V, K35D, K35I, K35L, K35M, K35N, K35P, K35Q, K35T, L36A, L36D, L36E, L36F, L36G, L36H, L36I, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M461, E61K, E61M, E61R, E62T, E62Y, K64D, K64E, K64G, K64L, K64Q, K64R, P65D, P65E, P65F, P65G, P65H, P651, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L94I, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q1261, and Q126V.

9. The isolated polypeptide of claim 8, wherein said substitution is H16D, L19D, L19E, L36D, L36P, L40D, L40G, F42E, F42R, E61R, P65Y, L72N, L80K, R81K, N88D, V91D, V91N, L94Y, E95D, E95G, Y107H, or Y107R.

10. The isolated polypeptide of claim 8, wherein said mutein further comprises a deletion of alanine at position 1 of SEQ ID NO:4.

11. The isolated polypeptide of claim 6, wherein said pro-inflammatory cytokine is TNF-α.

12. The isolated polypeptide of claim 6, wherein said mutein provides maintained or improved human NK cell-mediated natural killer cytotoxicity, lymphokine activated killer (LAK) cytotoxicity, or ADCC-mediated cytotoxicity relative to that observed for a similar amount of des-alanyl-1, C125S human IL-2 or C125S human IL-2 under comparable assay conditions, wherein said NK cell-mediated cytotoxicity is assayed using the NK3.3 cytotoxicity bioassay.

13. The isolated polypeptide of claim 6, wherein said mutein provides maintained or improved induction of phosphorylated AKT in the NK 3.3 cell line relative to that observed for a similar amount of des-alanyl 1 C125S human IL-2 or C125S human IL-2 under comparable assay conditions.

14. The isolated polypeptide of claim 6, wherein said NK cell proliferation induced by said mutein is greater than 150% of that induced by a similar amount of des-alanyl-1, C125S human IL-2 or C125S human IL-2 under comparable assay conditions.

15. The isolated polypeptide of claim 14, wherein said NK cell proliferation induced by said mutein is greater than 170% of that induced by des-alanyl-1, C125S human IL-2 or C125S human IL-2.

16. The isolated polypeptide of claim 15, wherein said NK cell proliferation induced by said mutein is about 200% to about 210% of that induced by des-alanyl-1, C125S human IL-2 or C125S human IL-2.

17. The isolated polypeptide of claim 6, wherein said NK cell proliferation induced by said mutein is increased by at least 10% over that induced by a similar amount of des-alanyl-1, C125S human IL-2 or C125S human IL-2 under comparable assay conditions.

18. The isolated polypeptide of claim 17, wherein said NK cell proliferation induced by said mutein is increased by at least 15% over that induced by des-alanyl-1, C125S human IL-2 or C125S human IL-2.

19. The isolated polypeptide of claim 18, wherein said pro-inflammatory cytokine production induced by said mutein is less than 100% of that induced by a similar amount of des-alanyl-1, C125S human IL-2 or C125S human IL-2 under similar assay conditions.

20. The isolated polypeptide of claim 19, wherein said pro-inflammatory cytokine production induced by said mutein is less than 70% of that induced by des-alanyl-1, C125S human IL-2 or C125S human IL-2.

21. An isolated polypeptide comprising a mutein of human IL-2, wherein said mutein comprises the amino acid sequence set forth in SEQ ID NO:4 with a serine substituted for cysteine at position 125 of SEQ ID NO:4 and at least one additional amino acid substitution within SEQ ID NO:4, wherein the ratio of IL-2-induced NK cell proliferation to IL-2-induced TNF-α production of said mutein is at least 1.5-fold greater than that observed for a similar amount of des-alanyl-1, C125S human IL-2 or C125S human IL-2 under comparable assay conditions, wherein NK cell proliferation at 0.1 nM mutein and TNF-α production at 1.0 nM mutein are assayed using the NK-92 bioassay.

22. The isolated polypeptide of claim 21, wherein said ratio is at least 2.5-fold greater than that observed for des-alanyl-1, C125S human IL-2 or C125S human IL-2.

23. The isolated polypeptide of claim 21, wherein said ratio is at least 3.0-fold greater than that observed for des-alanyl-1, C125S human IL-2 or C125S human IL-2.

24. The isolated polypeptide of claim 21, wherein said mutein further comprises a deletion of alanine at position 1 of SEQ ID NO:4.

25. An isolated polypeptide comprising an amino acid sequence for a mutein of human IL-2, wherein said mutein comprises the amino acid sequence set forth in SEQ ID NO:4 with a serine substituted for cysteine at position 125 of SEQ ID NO:4 and with at least one additional amino acid substitution at a position of SEQ ID NO:4 selected from the group consisting of positions 16, 36, 40, 42, 61, 65, 67, 72, 91, 94, 95, and 107.

26. The isolated polypeptide of claim 25, wherein said mutein further comprises a deletion of alanine at position 1 of SEQ ID NO:4.

27. A method of producing a mutein of human interleukin-2 (IL-2) that is capable of maintaining or enhancing proliferation of NK cells and which also induces a lower level of pro-inflammatory cytokine production by NK cells as compared with a similar amount of a reference human IL-2 mutein selected from des-alanyl-1, C125S human IL-2 and C125 human IL-2 under similar assay conditions, wherein said NK cell proliferation and pro-inflammatory cytokine production are assayed using the NK-92 bioassay, said method comprising:
a) transforming a host cell with an expression vector comprising a nucleic acid molecule of claim 1;
b) culturing said host cell in a cell culture medium under conditions that allow expression of said nucleic acid molecule as a polypeptide; and
c) isolating said polypeptide.

28. A method of producing a mutein of human interleukin-2 (IL-2) that is capable of maintaining or enhancing proliferation of NK cells and which also induces a lower level of pro-inflammatory cytokine production by NK cells as compared with a similar amount of a reference human IL-2 mutein selected from des-alanyl-1, C125S human IL-2 and C125S human IL-2 under similar assay conditions, wherein said NK cell proliferation and said pro-inflammatory cytokine production are assayed using the NK-92 bioassay, said method comprising:
a) transforming a host cell with an expression vector comprising a nucleic acid molecule encoding the polypeptide of claim 25;
b) culturing said host cell in a cell culture medium under conditions that allow expression of said nucleic acid molecule as a polypeptide; and
c) isolating said polypeptide.

29. A pharmaceutical composition comprising a therapeutically effective amount of a human IL-2 mutein of claim 2 and a pharmaceutically acceptable carrier.

30. A method for stimulating the immune system of an mammal, comprising administering to said mammal a therapeutically effective amount of a human IL-2 mutein, wherein said mutein induces a lower level of pro-inflammatory cytokine production by NK cells and maintains or enhances NK cell proliferation compared to a similar amount of a reference IL-2 molecule selected from des-alanyl-1, C1125S human IL-2 and C125S human IL-2 under comparable assay conditions, wherein said NK cell proliferation and said pro-inflammatory cytokine production are assayed using the NK-92 bioassay.

31. The method of claim 30, wherein said mammal is a human.

32. The method of claim 30, wherein said human IL-2 mutein comprises an amino acid sequence selected from the group consisting of:
a) the amino acid sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;
b) an amino acid sequence comprising residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;
c) the amino acid sequence of a) or b), wherein said sequence comprises an alanine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344; and
d) the amino acid sequence of a) or b), wherein said sequence comprises a cysteine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

33. A method for treating a cancer in a mammal, comprising administering to said mammal a therapeutically effective amount of a human IL-2 mutein, wherein said mutein induces a lower level of pro-inflammatory cytokine production by NK cells and maintains or enhances NK cell proliferation compared to a similar concentration of a reference IL-2 molecule selected from des-alanyl-1, C125S human IL-2 and C125S human IL-2 under similar assay conditions, wherein said NK cell proliferation and said pro-inflammatory cytokine production are assayed using the NK-92 bioassay.

34. The method of claim 33, wherein said mammal is a human.

35. The method of claim 33, wherein said human IL-2 mutein comprises an amino acid sequence selected from the group consisting of:
a) the amino acid sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;
b) an amino acid sequence comprising residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;
c) the amino acid sequence of a) or b), wherein said sequence comprises an alanine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344; and
d) the amino acid sequence of a) or b), wherein said sequence comprises a cysteine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

36. A method for reducing interleukin-2 (IL-2)-induced toxicity symptoms in a subject undergoing IL-2 administration as a treatment protocol, said method comprising administering said IL-2 as an IL-2 mutein, wherein said IL-2 mutein comprises an amino acid sequence selected from the group consisting of:
a) the amino acid sequence set forth in SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;
b) an amino acid sequence comprising residues 2-133 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344;
c) the amino acid sequence of a) or b), wherein said sequence comprises an alanine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344; and
d) the amino acid sequence of a) or b), wherein said sequence comprises a cysteine residue substituted for the serine residue at position 125 of SEQ ID NO:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, or 344.

37. The isolated polypeptide of claim 6, wherein said mutein has a higher maximum tolerated dose relative to that observed for des-alanyl-1, C125S human IL-2 or C125S human IL-2, wherein said maximum tolerated dose is determined using a B16F10 melanoma animal model.

38. The isolated polypeptide of claim 6, wherein said mutein shows comparable or improved anti-tumor activity and reduced adverse effects compared to treatment with des-alanyl-1, C125S human IL-2 or C125S human IL-2 under comparable treatment conditions, wherein said anti-tumor activity is evaluated using a B16F 10 melanoma animal model.

39. The isolated polypeptide of claim 6, wherein said mutein shows comparable or improved anti-tumor activity and reduced adverse effects compared to treatment with des-alanyl-1, C125S human IL-2 or C125S human IL-2 under comparable treatment conditions, wherein said anti-tumor activity is evaluated using a high grade non-Hodgkin's lymphoma Namalwa animal model or a low grade non-Hodgkin's lymphoma Daudi animal model.

40. The isolated polypeptide of claim 6, wherein said mutein when coadministered with rituximab shows comparable or improved anti-tumor activity and reduced adverse effects compared to treatment with des-alanyl-1, C125S human IL-2 or C125S human IL-2 under comparable treatment conditions, wherein said anti-tumor activity is evaluated using a high grade non-Hodgkin's lymphoma Namalwa animal model or a low grade non-Hodgkin's lymphoma Daudi animal model.

41. The isolated polypeptide of claim 39 or 40, wherein said mutein shows improved immune effector cell activation compared with a similar amount of des-alanyl-1, C125S human IL-2 or C125S human IL-2.

42. The isolated polypeptide of claim 41, wherein said mutein shows improved immune effector cell activation of a cell selected from the group consisting of a T cell, a NK cell, a monocyte, a macrophage, and a neutrophil.

43. The isolated polypeptide of claim 40, wherein said mutein shows improved antibody-dependent cellular cytotoxicity (ADCC)-mediated cytolytic killing compared with a similar amount of des-alanyl-1, C125S human IL-2 or C125S human IL-2.

44. The isolated polypeptide of claim 6, wherein said mutein causes less vascular leak as compared with a similar amount of des-alanyl-1, C125S human IL-2 or C125S human IL-2 in an animal model of vascular leak syndrome.

45. The isolated polypeptide of claim 6, wherein said mutein causes less change in body temperature as compared with a similar amount of des-alanyl-1, C 125 S human IL-2 or C125S human IL-2 in an animal model, wherein body temperature is monitored in said animal with a temperature chip.

46. The isolated polypeptide of claim 6, wherein said mutein demonstrates improved tolerability when administered to a subject as determined by measurement of body temperature using an in vivo temperature chip, measurement of vascular leak, or measurement of maximum tolerated dose in said subject.


SEQUENCE LISTING (346 sequences)

SEQ ID NO: 1 - DNA, 459 bp, Homo sapiens
  CDS (1)...(459)
  misc_feature (0)...(0): Human IL-2 precursor
atgtacaggatgcaactcctgtcttgcattgcactaagtcttgcacttgtcgcaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcagagcatcatctcaacactgact

SEQ ID NO: 2 - protein, 153 aa, Homo sapiens [sequence omitted]

SEQ ID NO: 3 - DNA, 399 bp, Homo sapiens
  CDS (1)...(399)
  misc_feature (0)...(0): Mature human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcagagcatcatctcaacactgact

SEQ ID NO: 4 - protein, 133 aa, Homo sapiens [sequence omitted]

SEQ ID NO: 5 - DNA, 399 bp, Artificial Sequence
  CDS (1)...(399): C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 6 - protein, 133 aa, Artificial Sequence
  C125S human IL-2
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 7 - DNA, 396 bp, Artificial Sequence
  CDS (1)...(396): des-alanyl-1, C125S human IL-2 mutein
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 8 - protein, 132 aa, Artificial Sequence
  des-alanyl-1, C125S human IL-2
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 9 - DNA, 399 bp, Artificial Sequence
  T7A, C125S human IL-2 mutein
  CDS (1)...(399)
gcacctacttcaagttctgctaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 10 - protein, 133 aa, Artificial Sequence
  T7A, C125S human IL-2
APTSSSAKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 11 - DNA, 399 bp, Artificial Sequence
  T7D, C125S human IL-2 mutein
  CDS (1)...(399)
gcacctacttcaagttctgataagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 12 - protein, 133 aa, Artificial Sequence
  T7D, C125S human IL-2
APTSSSDKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 13 - DNA, 399 bp, Artificial Sequence
  T7R, C125S human IL-2 mutein
  CDS (1)...(399)
gcacctacttcaagttctagaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 14 - protein, 133 aa, Artificial Sequence
  T7R, C125S human IL-2
APTSSSRKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 15 - DNA, 399 bp, Artificial Sequence
  K8L, C125S human IL-2 mutein
  CDS (1)...(399)
gcacctacttcaagttctacattgaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 16 - protein, 133 aa, Artificial Sequence
  K8L, C125S human IL-2 mutein
APTSSSTLKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 17 - DNA, 399 bp, Artificial Sequence
  K9A, C125S human IL-2 mutein
  CDS (1)...(399)
gcacctacttcaagttctacaaaggctacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 18 - protein, 133 aa, Artificial Sequence
  K9A, C125S human IL-2 mutein
APTSSSTKATQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 19 - DNA, 399 bp, Artificial Sequence
  K9D, C125S human IL-2 mutein
  CDS (1)...(399)
gcacctacttcaagttctacaaaggatacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 20 - protein, 133 aa, Artificial Sequence
  K9D, C125S human IL-2 mutein
APTSSSTKDTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 21 - DNA, 399 bp, Artificial Sequence
  K9R, C125S human IL-2 mutein
  CDS (1)...(399)
gcacctacttcaagttctacaaagagaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 22 - protein, 133 aa, Artificial Sequence
  K9R, C125S human IL-2 mutein
APTSSSTKRTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 23 - DNA, 399 bp, Artificial Sequence
  K9S, C125S human IL-2 mutein
  CDS (1)...(399)
gcacctacttcaagttctacaaagagtacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 24 - protein, 133 aa, Artificial Sequence
  K9S, C125S human IL-2 mutein
APTSSSTKSTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 25 - DNA, 399 bp, Artificial Sequence
  K9V, C125S human IL-2 mutein
  CDS (1)...(399)
gcacctacttcaagttctacaaaggttacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 26 - protein, 133 aa, Artificial Sequence
  K9V, C125S human IL-2 mutein
APTSSSTKVTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 27 - DNA, 399 bp, Artificial Sequence
  K9W, C125S human IL-2 mutein
  CDS (1)...(399)
gcacctacttcaagttctacaaagtggacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 28 - protein, 133 aa, Artificial Sequence
  K9W, C125S human IL-2 mutein
APTSSSTKWTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 29 - DNA, 399 bp, Artificial Sequence
  T10K, C125S human IL-2 mutein
  CDS (1)...(399)
gcacctacttcaagttctacaaagaaaaagcagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 30 - protein, 133 aa, Artificial Sequence
  T10K, C125S human IL-2 mutein
APTSSSTKKKQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 31 - DNA, 399 bp, Artificial Sequence
  T10N, C125S human IL-2 mutein
  CDS (1)...(399)
gcacctacttcaagttctacaaagaaaaaccagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 32 - protein, 133 aa, Artificial Sequence
  T10N, C125S human IL-2 mutein
APTSSSTKKNQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 33 - DNA, 399 bp, Artificial Sequence
  Q11A, C125S human IL-2 mutein
  CDS (1)...(399)
gcacctacttcaagttctacaaagaaaacagctctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 34 - protein, 133 aa, Artificial Sequence
  Q11A, C125S human IL-2 mutein
APTSSSTKKTALQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 35 - DNA, 399 bp, Artificial Sequence
  Q11R, C125S human IL-2 mutein
  CDS (1)...(399)
gcacctacttcaagttctacaaagaaaacaagactacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 36 - protein, 133 aa, Artificial Sequence
  Q11R, C125S human IL-2 mutein
APTSSSTKKTRLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 37 - DNA, 399 bp, Artificial Sequence
  Q11T, C125S human IL-2 mutein
  CDS (1)...(399)
gcacctacttcaagttctacaaagaaaacaacgctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 38 - protein, 133 aa, Artificial Sequence
  Q11T, C125S human IL-2 mutein
APTSSSTKKTTLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 39 - DNA, 399 bp, Artificial Sequence
  E15A, C125S human IL-2 mutein
  CDS (1)...(399)
gcacctacttcaagttctacaaagaaaacacagctacaactggctcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 40 - protein, 133 aa, Artificial Sequence
  E15A, C125S human IL-2 mutein
APTSSSTKKTQLQLAHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 41 - DNA, 399 bp, Artificial Sequence
  H16D, C125S human IL-2 mutein
  CDS (1)...(399)
gcacctacttcaagttctacaaagaaaacacagctacaactggaggatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 42 - protein, 133 aa, Artificial Sequence
  H16D, C125S human IL-2 mutein
APTSSSTKKTQLQLEDLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 43 - DNA, 399 bp, Artificial Sequence
  H16E, C125S human IL-2 mutein
  CDS (1)...(399)
gcacctacttcaagttctacaaagaaaacacagctacaactggaggaattactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 44 - protein, 133 aa, Artificial Sequence
  H16E, C125S human IL-2 mutein
APTSSSTKKTQLQLEELLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 45 - DNA, 399 bp, Artificial Sequence
  L19D, C125S human IL-2 mutein
  CDS (1)...(399)
gcacctacttcaagttctacaaagaaaacacagctacaactggagcatttactggatgatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 46 - protein, 133 aa, Artificial Sequence
  L19D, C125S human IL-2 mutein
APTSSSTKKTQLQLEHLLDDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 47 - DNA, 399 bp, Artificial Sequence
  L19E, C125S human IL-2 mutein
  CDS (1)...(399)
gcacctacttcaagttctacaaagaaaacacagctacaactggagcatttactggaagatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 48 - protein, 133 aa, Artificial Sequence
  L19E, C125S human IL-2 mutein
APTSSSTKKTQLQLEHLLEDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 49 - DNA, 399 bp, Artificial Sequence
  D20E, C125S human IL-2 mutein
  CDS (1)...(399)
gcacctacttcaagttctacaaagaaaacacagctacaactggagcatttactgctggaattacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 50 - protein, 133 aa, Artificial Sequence
  D20E, C125S human IL-2 mutein
APTSSSTKKTQLQLEHLLLELQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 51 - DNA, 399 bp, Artificial Sequence
  I24L, C125S human IL-2 mutein
  CDS (1)...(399)
gcacctacttcaagttctacaaagaaaacacagctacaactggagcatttactgctggatttacagatgttgttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 52 - protein, 133 aa, Artificial Sequence
  I24L, C125S human IL-2 mutein
APTSSSTKKTQLQLEHLLLDLQMLLNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 53 - DNA, 399 bp, Artificial Sequence
  K32A, C125S human IL-2 mutein
  CDS (1)...(399)
gcacctacttcaagttctacaaagaaaacacagctacaactggagcatttactgctggatttacagatgattttgaatggaattaataattacgctaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 54 - protein, 133 aa, Artificial Sequence
  K32A, C125S human IL-2 mutein
APTSSSTKKTQLQLEHLLLDLQMILNGINNYANPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 55 - DNA, 399 bp, Artificial Sequence
  K32W, C125S human IL-2 mutein
  CDS (1)...(399)
gcacctacttcaagttctacaaagaaaacacagctacaactggagcatttactgctggatttacagatgattttgaatggaattaataattactggaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 56 - protein, 133 aa, Artificial Sequence
  K32W, C125S human IL-2 mutein
APTSSSTKKTQLQLEHLLLDLQMILNGINNYWNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 57 - DNA, 399 bp, Artificial Sequence
  N33E, C125S human IL-2 mutein
  CDS (1)...(399)
gcacctacttcaagttctacaaagaaaacacagctacaactggagcatttactgctggatttacagatgattttgaatggaattaataattacaaggaacccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 58 - protein, 133 aa, Artificial Sequence
  N33E, C125S human IL-2 mutein
APTSSSTKKTQLQLEHLLLDLQMILNGINNYKEPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 59 - DNA, 399 bp, Artificial Sequence
  P34E, C125S human IL-2 mutein
  CDS (1)...(399)
gcacctacttcaagttctacaaagaaaacacagctacaactggagcatttactgctggatttacagatgattttgaatggaattaataattacaagaatgaaaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 60 - protein, 133 aa, Artificial Sequence
  P34E, C125S human IL-2 mutein
APTSSSTKKTQLQLEHLLLDLQMILNGINNYKNEKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 61 - DNA, 399 bp, Artificial Sequence
  P34R, C125S human IL-2 mutein
  CDS (1)...(399)
gcacctacttcaagttctacaaagaaaacacagctacaactggagcatttactgctggatttacagatgattttgaatggaattaataattacaagaatagaaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 62 - protein, 133 aa, Artificial Sequence
  P34R, C125S human IL-2 mutein
APTSSSTKKTQLQLEHLLLDLQMILNGINNYKNRKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 63 - DNA, 399 bp, Artificial Sequence
  P34S, C125S human IL-2 mutein
  CDS (1)...(399)
gcacctacttcaagttctacaaagaaaacacagctacaactggagcatttactgctggatttacagatgattttgaatggaattaataattacaagaatagtaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 64 - protein, 133 aa, Artificial Sequence
  P34S, C125S human IL-2 mutein
APTSSSTKKTQLQLEHLLLDLQMILNGINNYKNSKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 65 - DNA, 399 bp, Artificial Sequence
  P34T, C125S human IL-2 mutein
  CDS (1)...(399)
gcacctacttcaagttctacaaagaaaacacagctacaactggagcatttactgctggatttacagatgattttgaatggaattaataattacaagaatacgaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 66 - protein, 133 aa, Artificial Sequence
  P34T, C125S human IL-2 mutein
APTSSSTKKTQLQLEHLLLDLQMILNGINNYKNTKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 67 - DNA, 399 bp, Artificial Sequence
  P34V, C125S human IL-2 mutein
  CDS (1)...(399)
gcacctacttcaagttctacaaagaaaacacagctacaactggagcatttactgctggatttacagatgattttgaatggaattaataattacaagaatgttaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 68 - protein, 133 aa, Artificial Sequence
  P34V, C125S human IL-2 mutein
APTSSSTKKTQLQLEHLLLDLQMILNGINNYKNVKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 69 - DNA, 399 bp, Artificial Sequence
  K35D, C125S human IL-2 mutein
  CDS (1)...(399)
gcacctacttcaagttctacaaagaaaacacagctacaactggagcatttactgctggatttacagatgattttgaatggaattaataattacaagaatcccgatctcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 70 - protein, 133 aa, Artificial Sequence
  K35D, C125S human IL-2 mutein
APTSSSTKKTQLQLEHLLLDLQMILNGINNYKNPDLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 71 - DNA, 399 bp, Artificial Sequence
  K35I, C125S human IL-2 mutein
  CDS (1)...(399)
gcacctacttcaagttctacaaagaaaacacagctacaactggagcatttactgctggatttacagatgattttgaatggaattaataattacaagaatcccatactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 72 - protein, 133 aa, Artificial Sequence
  K35I, C125S human IL-2 mutein
APTSSSTKKTQLQLEHLLLDLQMILNGINNYKNPILTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 73 - DNA, 399 bp, Artificial Sequence
  K35L, C125S human IL-2 mutein
  CDS (1)...(399)
gcacctacttcaagttctacaaagaaaacacagctacaactggagcatttactgctggatttacagatgattttgaatggaattaataattacaagaatcccttgctcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 74 - protein, 133 aa, Artificial Sequence
  K35L, C125S human IL-2 mutein
APTSSSTKKTQLQLEHLLLDLQMILNGINNYKNPLLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 75 - DNA, 399 bp, Artificial Sequence
  K35M, C125S human IL-2 mutein
  CDS (1)...(399)
gcacctacttcaagttctacaaagaaaacacagctacaactggagcatttactgctggatttacagatgattttgaatggaattaataattacaagaatcccatgctcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 76 - protein, 133 aa, Artificial Sequence
  K35M, C125S human IL-2 mutein
APTSSSTKKTQLQLEHLLLDLQMILNGINNYKNPMLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 77 - DNA, 399 bp, Artificial Sequence
  K35N, C125S human IL-2 mutein
  CDS (1)...(399)
gcacctacttcaagttctacaaagaaaacacagctacaactggagcatttactgctggatttacagatgattttgaatggaattaataattacaagaatcccaacctcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 78 - protein, 133 aa, Artificial Sequence
  K35N, C125S human IL-2 mutein
APTSSSTKKTQLQLEHLLLDLQMILNGINNYKNPNLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 79 - DNA, 399 bp, Artificial Sequence
  K35P, C125S human IL-2 mutein
  CDS (1)...(399)
gcacctacttcaagttctacaaagaaaacacagctacaactggagcatttactgctggatttacagatgattttgaatggaattaataattacaagaatcccccactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 80 - protein, 133 aa, Artificial Sequence
  K35P, C125S human IL-2 mutein
APTSSSTKKTQLQLEHLLLDLQMILNGINNYKNPPLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 81 - DNA, 399 bp, Artificial Sequence
  K35Q, C125S human IL-2 mutein
  CDS (1)...(399)
gcacctacttcaagttctacaaagaaaacacagctacaactggagcatttactgctggatttacagatgattttgaatggaattaataattacaagaatccccaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 82 - protein, 133 aa, Artificial Sequence
  K35Q, C125S human IL-2 mutein
APTSSSTKKTQLQLEHLLLDLQMILNGINNYKNPQLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 83 - DNA, 399 bp, Artificial Sequence
  K35T, C125S human IL-2 mutein
  CDS (1)...(399)
gcacctacttcaagttctacaaagaaaacacagctacaactggagcatttactgctggatttacagatgattttgaatggaattaataattacaagaatcccacgctcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 84 - protein, 133 aa, Artificial Sequence
  K35T, C125S human IL-2 mutein
APTSSSTKKTQLQLEHLLLDLQMILNGINNYKNPTLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 85 - DNA, 399 bp, Artificial Sequence
  L36A, C125S human IL-2 mutein
  CDS (1)...(399)
gcacctacttcaagttctacaaagaaaacacagctacaactggagcatttactgctggatttacagatgattttgaatggaattaataattacaagaatcccaaagctaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 86 - protein, 133 aa, Artificial Sequence
  L36A, C125S human IL-2 mutein
APTSSSTKKTQLQLEHLLLDLQMILNGINNYKNPKATRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 87 - DNA, 399 bp, Artificial Sequence
  L36D, C125S human IL-2 mutein
  CDS (1)...(399)
gcacctacttcaagttctacaaagaaaacacagctacaactggagcatttactgctggatttacagatgattttgaatggaattaataattacaagaatcccaaagacaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 88 - protein, 133 aa, Artificial Sequence
  L36D, C125S human IL-2 mutein
APTSSSTKKTQLQLEHLLLDLQMILNGINNYKNPKDTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 89 - DNA, 399 bp, Artificial Sequence
  L36E, C125S human IL-2 mutein
  CDS (1)...(399)
gcacctacttcaagttctacaaagaaaacacagctacaactggagcatttactgctggatttacagatgattttgaatggaattaataattacaagaatcccaaagaaaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 90 - protein, 133 aa, Artificial Sequence
  L36E, C125S human IL-2 mutein
APTSSSTKKTQLQLEHLLLDLQMILNGINNYKNPKETRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 91 - DNA, 399 bp, Artificial Sequence
  L36F, C125S human IL-2 mutein
  CDS (1)...(399)
gcacctacttcaagttctacaaagaaaacacagctacaactggagcatttactgctggatttacagatgattttgaatggaattaataattacaagaatcccaaattcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 92 - protein, 133 aa, Artificial Sequence
  L36F, C125S human IL-2 mutein
APTSSSTKKTQLQLEHLLLDLQMILNGINNYKNPKFTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 93 - DNA, 399 bp, Artificial Sequence
  L36G, C125S human IL-2 mutein
  CDS (1)...(399)
gcacctacttcaagttctacaaagaaaacacagctacaactggagcatttactgctggatttacagatgattttgaatggaattaataattacaagaatcccaaaggtaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 94 - protein, 133 aa, Artificial Sequence
  L36G, C125S human IL-2 mutein
APTSSSTKKTQLQLEHLLLDLQMILNGINNYKNPKGTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 95 - DNA, 399 bp, Artificial Sequence
  L36H, C125S human IL-2 mutein
  CDS (1)...(399)
gcacctacttcaagttctacaaagaaaacacagctacaactggagcatttactgctggatttacagatgattttgaatggaattaataattacaagaatcccaaacacaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 96 - protein, 133 aa, Artificial Sequence
  L36H, C125S human IL-2 mutein
APTSSSTKKTQLQLEHLLLDLQMILNGINNYKNPKHTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 97 - DNA, 399 bp, Artificial Sequence
  L36I, C125S human IL-2 mutein
  CDS (1)...(399)
gcacctacttcaagttctacaaagaaaacacagctacaactggagcatttactgctggatttacagatgattttgaatggaattaataattacaagaatcccaaaataaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 98 - protein, 133 aa, Artificial Sequence
  L36I, C125S human IL-2 mutein
APTSSSTKKTQLQLEHLLLDLQMILNGINNYKNPKITRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 99 - DNA, 399 bp, Artificial Sequence
  L36K, C125S human IL-2 mutein
  CDS (1)...(399)
gcacctacttcaagttctacaaagaaaacacagctacaactggagcatttactgctggatttacagatgattttgaatggaattaataattacaagaatcccaaaaagaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 100 - protein, 133 aa, Artificial Sequence
  L36K, C125S human IL-2 mutein
APTSSSTKKTQLQLEHLLLDLQMILNGINNYKNPKKTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 101 - DNA, 399 bp, Artificial Sequence
  L36M, C125S human IL-2 mutein
  CDS (1)...(399)
gcacctacttcaagttctacaaagaaaacacagctacaactggagcatttactgctggatttacagatgattttgaatggaattaataattacaagaatcccaaaatgaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 102 - protein, 133 aa, Artificial Sequence
  L36M, C125S human IL-2 mutein
APTSSSTKKTQLQLEHLLLDLQMILNGINNYKNPKMTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 103 - DNA, 399 bp, Artificial Sequence
  L36N, C125S human IL-2 mutein
  CDS (1)...(399)
gcacctacttcaagttctacaaagaaaacacagctacaactggagcatttactgctggatttacagatgattttgaatggaattaataattacaagaatcccaaaaacaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 104 - protein, 133 aa, Artificial Sequence
  L36N, C125S human IL-2 mutein
APTSSSTKKTQLQLEHLLLDLQMILNGINNYKNPKNTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 105 - DNA, 399 bp, Artificial Sequence
  L36P, C125S human IL-2 mutein
  CDS (1)...(399)
gcacctacttcaagttctacaaagaaaacacagctacaactggagcatttactgctggatttacagatgattttgaatggaattaataattacaagaatcccaaaccaaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 106 - protein, 133 aa, Artificial Sequence
  L36P, C125S human IL-2 mutein
APTSSSTKKTQLQLEHLLLDLQMILNGINNYKNPKPTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 107 - DNA, 399 bp, Artificial Sequence
  L36R, C125S human IL-2 mutein
  CDS (1)...(399)
gcacctacttcaagttctacaaagaaaacacagctacaactggagcatttactgctggatttacagatgattttgaatggaattaataattacaagaatcccaaaagaaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 108 - protein, 133 aa, Artificial Sequence
  L36R, C125S human IL-2 mutein
APTSSSTKKTQLQLEHLLLDLQMILNGINNYKNPKRTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 109 - DNA, 399 bp, Artificial Sequence
  L36S, C125S human IL-2 mutein
  CDS (1)...(399)
gcacctacttcaagttctacaaagaaaacacagctacaactggagcatttactgctggatttacagatgattttgaatggaattaataattacaagaatcccaaaagtaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 110 - protein, 133 aa, Artificial Sequence
  L36S, C125S human IL-2 mutein
APTSSSTKKTQLQLEHLLLDLQMILNGINNYKNPKSTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 111 - DNA, 399 bp, Artificial Sequence
  L36W, C125S human IL-2 mutein
  CDS (1)...(399)
gcacctacttcaagttctacaaagaaaacacagctacaactggagcatttactgctggatttacagatgattttgaatggaattaataattacaagaatcccaaatggaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 112 - protein, 133 aa, Artificial Sequence
  L36W, C125S human IL-2 mutein
APTSSSTKKTQLQLEHLLLDLQMILNGINNYKNPKWTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 113 - DNA, 399 bp, Artificial Sequence
  L36Y, C125S human IL-2 mutein
  CDS (1)...(399)
gcacctacttcaagttctacaaagaaaacacagctacaactggagcatttactgctggatttacagatgattttgaatggaattaataattacaagaatcccaaatacaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 114 - protein, 133 aa, Artificial Sequence
  L36Y, C125S human IL-2 mutein
APTSSSTKKTQLQLEHLLLDLQMILNGINNYKNPKYTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 115 - DNA, 399 bp, Artificial Sequence
  R38D, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccgacatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 116 - protein, 133 aa, Artificial Sequence
  R38D, C125S human IL-2 mutein
APTSSSTKKTQLQLEHLLLDLQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 117 - DNA, 399 bp, Artificial Sequence
  R38G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccggtatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 118 - protein, 133 aa, Artificial Sequence
  R38G, C125S human IL-2 mutein
APTSSSTKKTQLQLEHLLLDLQMILNGINNYKNPKLTGMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 119 - DNA, 399 bp, Artificial Sequence
  R38N, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaacatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 120 - protein, 133 aa, Artificial Sequence
  R38N, C125S human IL-2 mutein
APTSSSTKKTQLQLEHLLLDLQMILNGINNYKNPKLTNMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 121 - DNA, 399 bp, Artificial Sequence
  R38P, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccccaatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 122 - protein, 133 aa, Artificial Sequence
  R38P, C125S human IL-2 mutein
APTSSSTKKTQLQLEHLLLDLQMILNGINNYKNPKLTPMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 123 - DNA, 399 bp, Artificial Sequence
  R38S, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccagtatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 124 - protein, 133 aa, Artificial Sequence
  R38S, C125S human IL-2 mutein
APTSSSTKKTQLQLEHLLLDLQMILNGINNYKNPKLTSMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 125 - DNA, 399 bp, Artificial Sequence
  L40D, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatggacacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 126 - protein, 133 aa, Artificial Sequence
  L40D, C125S human IL-2 mutein
APTSSSTKKTQLQLEHLLLDLQMILNGINNYKNPKLTRMDTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 127 - DNA, 399 bp, Artificial Sequence
  L40G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgggtacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 128 - protein, 133 aa, Artificial Sequence
  L40G, C125S human IL-2 mutein
APTSSSTKKTQLQLEHLLLDLQMILNGINNYKNPKLTRMGTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 129 - DNA, 399 bp, Artificial Sequence
  L40N, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgaacacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 130 - protein, 133 aa, Artificial Sequence
  L40N, C125S human IL-2 mutein
APTSSSTKKTQLQLEHLLLDLQMILNGINNYKNPKLTRMNTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 131 - DNA, 399 bp, Artificial Sequence
  L40S, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgagtacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 132 - protein, 133 aa, Artificial Sequence
  L40S, C125S human IL-2 mutein
APTSSSTKKTQLQLEHLLLDLQMILNGINNYKNPKLTRMSTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 133 - DNA, 399 bp, Artificial Sequence
  T41E, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcgaatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 134 - protein, 133 aa, Artificial Sequence
  T41E, C125S human IL-2 mutein
APTSSSTKKTQLQLEHLLLDLQMILNGINNYKNPKLTRMLEFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 135 - DNA, 399 bp, Artificial Sequence
  T41G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcggttttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 136 - protein, 133 aa, Artificial Sequence
  T41G, C125S human IL-2 mutein
APTSSSTKKTQLQLEHLLLDLQMILNGINNYKNPKLTRMLGFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 137 - DNA, 399 bp, Artificial Sequence
  F42A, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacagctaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 138 - protein, 133 aa, Artificial Sequence
  F42A, C125S human IL-2 mutein
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 139 - DNA, 399 bp, Artificial Sequence
  F42E, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacagaaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 140 - protein, 133 aa, Artificial Sequence
  F42E, C125S human IL-2 mutein
APTSSSTKKTQLQLEHLLLDLQMILNGINNYKNPKLTRMLTEKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 141 - DNA, 399 bp, Artificial Sequence
  F42R, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacaagaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 142 - protein, 133 aa, Artificial Sequence
  F42R, C125S human IL-2 mutein
APTSSSTKKTQLQLEHLLLDLQMILNGINNYKNPKLTRMLTRKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 143 - DNA, 399 bp, Artificial Sequence
  F42T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacaacgaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 144 - protein, 133 aa, Artificial Sequence
  F42T, C125S human IL-2 mutein
APTSSSTKKTQLQLEHLLLDLQMILNGINNYKNPKLTRMLTTKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 145 - DNA, 399 bp, Artificial Sequence
  F42V, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacagttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 146 - protein, 133 aa, Artificial Sequence
  F42V, C125S human IL-2 mutein
APTSSSTKKTQLQLEHLLLDLQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 147 - DNA, 399 bp, Artificial Sequence
  K43H, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttcacttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 148 - protein, 133 aa, Artificial Sequence
  K43H, C125S human IL-2 mutein
APTSSSTKKTQLQLEHLLLDLQMILNGINNYKNPKLTRMLTFHFYMPKKATELKHLQCLEEELKPLEEVLNLAQSKNFHLRPRDLISNINVIVLELKGSETTFMCEYADETATIVEFLNRWITFSQSIISTLT

SEQ ID NO: 149 - DNA, 399 bp, Artificial Sequence
  F44K, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagaagtacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 150 - protein, 133 aa, Artificial Sequence
  F44K, C125S human IL-2 mutein
APTSSSTKKTQLQLEHLLLDLQMILNGINNYKNPKLTRMLTFKKYMPKKATELKHLQCLEEELKPLEEVLNLAQSKNFHLRPRDLISNINVIVLELKGSETTFMCEYADETATIVEFLNRWITFSQSIISTLT

SEQ ID NO: 151 - DNA, 399 bp, Artificial Sequence
  M46I, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatacccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 152 - protein, 133 aa, Artificial Sequence
  M46I, C125S human IL-2 mutein
APTSSSTKKTQLQLEHLLLDLQMILNGINNYKNPKLTRMLTFKFYIPKKATELKHLQCLEEELKPLEEVLNLAQSKNFHLRPRDLISNINVIVLELKGSETTFMCEYADETATIVEFLNRWITFSQSIISTLT

SEQ ID NO: 153 - DNA, 399 bp, Artificial Sequence
  E61K, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaaaaggaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 154 - protein, 133 aa, Artificial Sequence
  E61K, C125S human IL-2 mutein
APTSSSTKKTQLQLEHLLLDLQMILNGINNYKNPKLTRMLTFKFYMPKKATELKHLQCLEKELKPLEEVLNLAQSKNFHLRPRDLISNINVIVLELKGSETTFMCEYADETATIVEFLNRWITFSQSIISTLT

SEQ ID NO: 155 - DNA, 399 bp, Artificial Sequence
  E61M, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaaatggaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 156 - protein, 133 aa, Artificial Sequence
  E61M, C125S human IL-2 mutein
APTSSSTKKTQLQLEHLLLDLQMILNGINNYKNPKLTRMLTFKFYMPKKATELKHLQCLEMELKPLEEVLNLAQSKNFHLRPRDLISNINVIVLELKGSETTFMCEYADETATIVEFLNRWITFSQSIISTLT

SEQ ID NO: 157 - DNA, 399 bp, Artificial Sequence
  E61R, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaaagagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 158 - protein, 133 aa, Artificial Sequence
  E61R, C125S human IL-2 mutein
APTSSSTKKTQLQLEHLLLDLQMILNGINNYKNPKLTRMLTFKFYMPKKATELKHLQCLERELKPLEEVLNLAQSKNFHLRPRDLISNINVIVLELKGSETTFMCEYADETATIVEFLNRWITFSQSIISTLT

SEQ ID NO: 159 - DNA, 399 bp, Artificial Sequence
  E62T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaaacgctcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 160 - protein, 133 aa, Artificial Sequence
  E62T, C125S human IL-2 mutein
APTSSSTKKTQLQLEHLLLDLQMILNGINNYKNPKLTRMLTFKFYMPKKATELKHLQCLEETLKPLEEVLNLAQSKNFHLRPRDLISNINVIVLELKGSETTFMCEYADETATIVEFLNRWITFSQSIISTLT

SEQ ID NO: 161 - DNA, 399 bp, Artificial Sequence
  E62Y, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaatacctcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 162 - protein, 133 aa, Artificial Sequence
  E62Y, C125S human IL-2 mutein
APTSSSTKKTQLQLEHLLLDLQMILNGINNYKNPKLTRMLTFKFYMPKKATELKHLQCLEEYLKPLEEVLNLAQSKNFHLRPRDLISNINVIVLELKGSETTFMCEYADETATIVEFLNRWITFSQSIISTLT

SEQ ID NO: 163 - DNA, 399 bp, Artificial Sequence
  K64D, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcgatcctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 164 - protein, 133 aa, Artificial Sequence
  K64D, C125S human IL-2 mutein
APTSSSTKKTQLQLEHLLLDLQMILNGINNYKNPKLTRMLTFKFYMPKKATELKHLQCLEEELDPLEEVLNLAQSKNFHLRPRDLISNINVIVLELKGSETTFMCEYADETATIVEFLNRWITFSQSIISTLT

SEQ ID NO: 165 - DNA, 399 bp, Artificial Sequence
  K64E, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcgaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 166 - protein, 133 aa, Artificial Sequence
  K64E, C125S human IL-2 mutein
APTSSSTKKTQLQLEHLLLDLQMILNGINNYKNPKLTRMLTFKFYMPKKATELKHLQCLEEELEPLEEVLNLAQSKNFHLRPRDLISNINVIVLELKGSETTFMCEYADETATIVEFLNRWITFSQSIISTLT

SEQ ID NO: 167 - DNA, 399 bp, Artificial Sequence
  K64G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcggtcctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 168 - protein, 133 aa, Artificial Sequence
  K64G, C125S human IL-2 mutein
APTSSSTKKTQLQLEHLLLDLQMILNGINNYKNPKLTRMLTFKFYMPKKATELKHLQCLEEELGPLEEVLNLAQSKNFHLRPRDLISNINVIVLELKGSETTFMCEYADETATIVEFLNRWITFSQSIISTLT

SEQ ID NO: 169 - DNA, 399 bp, Artificial Sequence
  K64L, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcttgcctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 170 - protein, 133 aa, Artificial Sequence
  K64L, C125S human IL-2 mutein
APTSSSTKKTQLQLEHLLLDLQMILNGINNYKNPKLTRMLTFKFYMPKKATELKHLQCLEEELLPLEEVLNLAQSKNFHLRPRDLISNINVIVLELKGSETTFMCEYADETATIVEFLNRWITFSQSIISTLT

SEQ ID NO: 171 - DNA, 399 bp, Artificial Sequence
  K64Q, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactccaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 172 - protein, 133 aa, Artificial Sequence
  K64Q, C125S human IL-2 mutein
APTSSSTKKTQLQLEHLLLDLQMILNGINNYKNPKLTRMLTFKFYMPKKATELKHLQCLEEELQPLEEVLNLAQSKNFHLRPRDLISNINVIVLELKGSETTFMCEYADETATIVEFLNRWITFSQSIISTLT

SEQ ID NO: 173 - DNA, 399 bp, Artificial Sequence
  K64R, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcagacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 174 - protein, 133 aa, Artificial Sequence
  K64R, C125S human IL-2 mutein
APTSSSTKKTQLQLEHLLLDLQMILNGINNYKNPKLTRMLTFKFYMPKKATELKHLQCLEEELRPLEEVLNLAQSKNFHLRPRDLISNINVIVLELKGSETTFMCEYADETATIVEFLNRWITFSQSIISTLT

SEQ ID NO: 175 - DNA, 399 bp, Artificial Sequence
  P65D, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaagatctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 176 - protein, 133 aa, Artificial Sequence
  P65D, C125S human IL-2 mutein
APTSSSTKKTQLQLEHLLLDLQMILNGINNYKNPKLTRMLTFKFYMPKKATELKHLQCLEEELKDLEEVLNLAQSKNFHLRPRDLISNINVIVLELKGSETTFMCEYADETATIVEFLNRWITFSQSIISTLT

SEQ ID NO: 177 - DNA, 399 bp, Artificial Sequence
  P65E, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaagaactggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 178 - protein, 133 aa, Artificial Sequence
  P65E, C125S human IL-2 mutein
APTSSSTKKTQLQLEHLLLDLQMILNGINNYKNPKLTRMLTFKFYMPKKATELKHLQCLEEELKELEEVLNLAQSKNFHLRPRDLISNINVIVLELKGSETTFMCEYADETATIVEFLNRWITFSQSIISTLT

SEQ ID NO: 179 - DNA, 399 bp, Artificial Sequence
  CDS (1)...(399)
  214: FEATURE:
  215: OTHER INFORMATION: P65F, C125S human IL-2 mutein
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaattcctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 180 - protein, 133 aa, Artificial Sequence
  P65F, C125S human IL-2 mutein
APTSSSTKKTQLQLEHLLLDLQMILNGINNYKNPKLTRMLTFKFYMPKKATELKHLQCLEEELKFLEEVLNLAQSKNFHLRPRDLISNINVIVLELKGSETTFMCEYADETATIVEFLNRWITFSQSIISTLT

SEQ ID NO: 181 - DNA, 399 bp, Artificial Sequence
  P65G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaaggtctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 182 - protein, 133 aa, Artificial Sequence
  P65G, C125S human IL-2 mutein
APTSSSTKKTQLQLEHLLLDLQMILNGINNYKNPKLTRMLTFKFYMPKKATELKHLQCLEEELKGLEEVLNLAQSKNFHLRPRDLISNINVIVLELKGSETTFMCEYADETATIVEFLNRWITFSQSIISTLT

SEQ ID NO: 183 - DNA, 399 bp, Artificial Sequence
  P65H, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacacctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 184 - protein, 133 aa, Artificial Sequence
  P65H, C125S human IL-2 mutein
APTSSSTKKTQLQLEHLLLDLQMILNGINNYKNPKLTRMLTFKFYMPKKATELKHLQCLEEELKHLEEVLNLAQSKNFHLRPRDLISNINVIVLELKGSETTFMCEYADETATIVEFLNRWITFSQSIISTLT

SEQ ID NO: 185 - DNA, 399 bp, Artificial Sequence
  P65I, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaaatactggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 186 - protein, 133 aa, Artificial Sequence
  P65I, C125S human IL-2 mutein
APTSSSTKKTQLQLEHLLLDLQMILNGINNYKNPKLTRMLTFKFYMPKKATELKHLQCLEEELKILEEVLNLAQSKNFHLRPRDLISNINVIVLELKGSETTFMCEYADETATIVEFLNRWITFSQSIISTLT

SEQ ID NO: 187 - DNA, 399 bp, Artificial Sequence
  P65K, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaaaagctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 188 - protein, 133 aa, Artificial Sequence
  P65K, C125S human IL-2 mutein
APTSSSTKKTQLQLEHLLLDLQMILNGINNYKNPKLTRMLTFKFYMPKKATELKHLQCLEEELKKLEEVLNLAQSKNFHLRPRDLISNINVIVLELKGSETTFMCEYADETATIVEFLNRWITFSQSIISTLT

SEQ ID NO: 189 - DNA, 399 bp, Artificial Sequence
  P65L, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaattgctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 190 - protein, 133 aa, Artificial Sequence
  P65L, C125S human IL-2 mutein
APTSSSTKKTQLQLEHLLLDLQMILNGINNYKNPKLTRMLTFKFYMPKKATELKHLQCLEEELKLLEEVLNLAQSKNFHLRPRDLISNINVIVLELKGSETTFMCEYADETATIVEFLNRWITFSQSIISTLT

SEQ ID NO: 191 - DNA, 399 bp, Artificial Sequence
  P65N, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaaaacctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 192 - protein, 133 aa, Artificial Sequence
  P65N, C125S human IL-2 mutein
APTSSSTKKTQLQLEHLLLDLQMILNGINNYKNPKLTRMLTFKFYMPKKATELKHLQCLEEELKNLEEVLNLAQSKNFHLRPRDLISNINVIVLELKGSETTFMCEYADETATIVEFLNRWITFSQSIISTLT

SEQ ID NO: 193 - DNA, 399 bp, Artificial Sequence
  P65Q, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacaactggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 194 - protein, 133 aa, Artificial Sequence
  P65Q, C125S human IL-2 mutein
APTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFSQSIISTLT

SEQ ID NO: 195 - DNA, 399 bp, Artificial Sequence
  P65R, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaaagactggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 196 - protein, 133 aa, Artificial Sequence
  P65R, C125S human IL-2 mutein
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFSQSIISTLT

SEQ ID NO: 197 - DNA, 399 bp, Artificial Sequence
  P65S, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaaagtctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 198 - protein, 133 aa, Artificial Sequence
  P65S, C125S human IL-2 mutein
APTSSSTKKTQLQLEHLLLDLQMILNGINNYKNPKLTRMLTFKFYMPKKATELKHLQCLEEELKSLEEVLNLAQSKNFHLRPRDLISNINVIVLELKGSETTFMCEYADETATIVEFLNRWITFSQSIISTLT

SEQ ID NO: 199 - DNA, 399 bp, Artificial Sequence
  P65T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaaacgctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 200 - protein, 133 aa, Artificial Sequence
  P65T, C125S human IL-2 mutein
APTSSSTKKTQLQLEHLLLDLQMILNGINNYKNPKLTRMLTFKFYMPKKATELKHLQCLEEELKTLEEVLNLAQSKNFHLRPRDLISNINVIVLELKGSETTFMCEYADETATIVEFLNRWITFSQSIISTLT

SEQ ID NO: 201 - DNA, 399 bp, Artificial Sequence
  P65V, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaagttctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 202 - protein, 133 aa, Artificial Sequence
  P65V, C125S human IL-2 mutein
APTSSSTKKTQLQLEHLLLDLQMILNGINNYKNPKLTRMLTFKFYMPKKATELKHLQCLEEELKVLEEVLNLAQSKNFHLRPRDLISNINVIVLELKGSETTFMCEYADETATIVEFLNRWITFSQSIISTLT

SEQ ID NO: 203 - DNA, 399 bp, Artificial Sequence
  P65W, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaatggctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 204 - protein, 133 aa, Artificial Sequence
  P65W, C125S human IL-2 mutein
APTSSSTKKTQLQLEHLLLDLQMILNGINNYKNPKLTRMLTFKFYMPKKATELKHLQCLEEELKWLEEVLNLAQSKNFHLRPRDLISNINVIVLELKGSETTFMCEYADETATIVEFLNRWITFSQSIISTLT

SEQ ID NO: 205 - DNA, 399 bp, Artificial Sequence
  P65Y, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaatacctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 206 - protein, 133 aa, Artificial Sequence
  P65Y, C125S human IL-2 mutein
APTSSSTKKTQLQLEHLLLDLQMILNGINNYKNPKLTRMLTFKFYMPKKATELKHLQCLEEELKYLEEVLNLAQSKNFHLRPRDLISNINVIVLELKGSETTFMCEYADETATIVEFLNRWITFSQSIISTLT

SEQ ID NO: 207 - DNA, 399 bp, Artificial Sequence
  L66A, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctgctgaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 208 - protein, 133 aa, Artificial Sequence
  L66A, C125S human IL-2 mutein
APTSSSTKKTQLQLEHLLLDLQMILNGINNYKNPKLTRMLTFKFYMPKKATELKHLQCLEEELKPAEEVLNLAQSKNFHLRPRDLISNINVIVLELKGSETTFMCEYADETATIVEFLNRWITFSQSIISTLT

SEQ ID NO: 209 - DNA, 399 bp, Artificial Sequence
  L66F, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctttcgaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 210 - protein, 133 aa, Artificial Sequence
  L66F, C125S human IL-2 mutein
APTSSSTKKTQLQLEHLLLDLQMILNGINNYKNPKLTRMLTFKFYMPKKATELKHLQCLEEELKPFEEVLNLAQSKNFHLRPRDLISNINVIVLELKGSETTFMCEYADETATIVEFLNRWITFSQSIISTLT

SEQ ID NO: 211 - DNA, 399 bp, Artificial Sequence
  E67A, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggctgaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 212 - protein, 133 aa, Artificial Sequence
  E67A, C125S human IL-2 mutein
APTSSSTKKTQLQLEHLLLDLQMILNGINNYKNPKLTRMLTFKFYMPKKATELKHLQCLEEELKPLAEVLNLAQSKNFHLRPRDLISNINVIVLELKGSETTFMCEYADETATIVEFLNRWITFSQSIISTLT

SEQ ID NO: 213 - DNA, 399 bp, Artificial Sequence
  L72G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatggtgctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 214 - protein, 133 aa, Artificial Sequence
  L72G, C125S human IL-2 mutein
APTSSSTKKTQLQLEHLLLDLQMILNGINNYKNPKLTRMLTFKFYMPKKATELKHLQCLEEELKPLEEVLNGAQSKNFHLRPRDLISNINVIVLELKGSETTFMCEYADETATIVEFLNRWITFSQSIISTLT

SEQ ID NO: 215 - DNA, 399 bp, Artificial Sequence
  L72N, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaataacgctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 216 - protein, 133 aa, Artificial Sequence
  L72N, C125S human IL-2 mutein
APTSSSTKKTQLQLEHLLLDLQMILNGINNYKNPKLTRMLTFKFYMPKKATELKHLQCLEEELKPLEEVLNNAQSKNFHLRPRDLISNINVIVLELKGSETTFMCEYADETATIVEFLNRWITFSQSIISTLT

SEQ ID NO: 217 - DNA, 399 bp, Artificial Sequence
  L72T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatacggctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 218 - protein, 133 aa, Artificial Sequence
  L72T, C125S human IL-2 mutein
APTSSSTKKTQLQLEHLLLDLQMILNGINNYKNPKLTRMLTFKFYMPKKATELKHLQCLEEELKPLEEVLNTAQSKNFHLRPRDLISNINVIVLELKGSETTFMCEYADETATIVEFLNRWITFSQSIISTLT

SEQ ID NO: 219 - DNA, 399 bp, Artificial Sequence
  F78S, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaacagtcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 220 - protein, 133 aa, Artificial Sequence
  F78S, C125S human IL-2 mutein
APTSSSTKKTQLQLEHLLLDLQMILNGINNYKNPKLTRMLTFKFYMPKKATELKHLQCLEEELKPLEEVLNLAQSKNSHLRPRDLISNINVIVLELKGSETTFMCEYADETATIVEFLNRWITFSQSIISTLT

SEQ ID NO: 221 - DNA, 399 bp, Artificial Sequence
  F78W, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactggcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 222 - protein, 133 aa, Artificial Sequence
  F78W, C125S human IL-2 mutein
APTSSSTKKTQLQLEHLLLDLQMILNGINNYKNPKLTRMLTFKFYMPKKATELKHLQCLEEELKPLEEVLNLAQSKNWHLRPRDLISNINVIVLELKGSETTFMCEYADETATIVEFLNRWITFSQSIISTLT

SEQ ID NO: 223 - DNA, 399 bp, Artificial Sequence
  H79F, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttttcttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 224 - protein, 133 aa, Artificial Sequence
  H79F, C125S human IL-2 mutein
APTSSSTKKTQLQLEHLLLDLQMILNGINNYKNPKLTRMLTFKFYMPKKATELKHLQCLEEELKPLEEVLNLAQSKNFFLRPRDLISNINVIVLELKGSETTFMCEYADETATIVEFLNRWITFSQSIISTLT

SEQ ID NO: 225 - DNA, 399 bp, Artificial Sequence
  H79M, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttatgttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 226 - protein, 133 aa, Artificial Sequence
  H79M, C125S human IL-2 mutein
APTSSSTKKTQLQLEHLLLDLQMILNGINNYKNPKLTRMLTFKFYMPKKATELKHLQCLEEELKPLEEVLNLAQSKNFMLRPRDLISNINVIVLELKGSETTFMCEYADETATIVEFLNRWITFSQSIISTLT

SEQ ID NO: 227 - DNA, 399 bp, Artificial Sequence
  H79N, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttaacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 228 - protein, 133 aa, Artificial Sequence
  H79N, C125S human IL-2 mutein
APTSSSTKKTQLQLEHLLLDLQMILNGINNYKNPKLTRMLTFKFYMPKKATELKHLQCLEEELKPLEEVLNLAQSKNFNLRPRDLISNINVIVLELKGSETTFMCEYADETATIVEFLNRWITFSQSIISTLT

SEQ ID NO: 229 - DNA, 399 bp, Artificial Sequence
  H79P, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttccattaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 230 - protein, 133 aa, Artificial Sequence
  H79P, C125S human IL-2 mutein
APTSSSTKKTQLQLEHLLLDLQMILNGINNYKNPKLTRMLTFKFYMPKKATELKHLQCLEEELKPLEEVLNLAQSKNFPLRPRDLISNINVIVLELKGSETTFMCEYADETATIVEFLNRWITFSQSIISTLT

SEQ ID NO: 231 - DNA, 399 bp, Artificial Sequence
  H79Q, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcaattaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 232 - protein, 133 aa, Artificial Sequence
  H79Q, C125S human IL-2 mutein
APTSSSTKKTQLQLEHLLLDLQMILNGINNYKNPKLTRMLTFKFYMPKKATELKHLQCLEEELKPLEEVLNLAQSKNFQLRPRDLISNINVIVLELKGSETTFMCEYADETATIVEFLNRWITFSQSIISTLT

SEQ ID NO: 233 - DNA, 399 bp, Artificial Sequence
  H79S, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttagtttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 234 - protein, 133 aa, Artificial Sequence
  H79S, C125S human IL-2 mutein
APTSSSTKKTQLQLEHLLLDLQMILNGINNYKNPKLTRMLTFKFYMPKKATELKHLQCLEEELKPLEEVLNLAQSKNFSLRPRDLISNINVIVLELKGSETTFMCEYADETATIVEFLNRWITFSQSIISTLT

SEQ ID NO: 235 - DNA, 399 bp, Artificial Sequence
  H79V, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttgttttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 236 - protein, 133 aa, Artificial Sequence
  H79V, C125S human IL-2 mutein
APTSSSTKKTQLQLEHLLLDLQMILNGINNYKNPKLTRMLTFKFYMPKKATELKHLQCLEEELKPLEEVLNLAQSKNFVLRPRDLISNINVIVLELKGSETTFMCEYADETATIVEFLNRWITFSQSIISTLT

SEQ ID NO: 237 - DNA, 399 bp, Artificial Sequence
  L80E, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacgaaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 238 - protein, 133 aa, Artificial Sequence
  L80E, C125S human IL-2 mutein
APTSSSTKKTQLQLEHLLLDLQMILNGINNYKNPKLTRMLTFKFYMPKKATELKHLQCLEEELKPLEEVLNLAQSKNFHERPRDLISNINVIVLELKGSETTFMCEYADETATIVEFLNRWITFSQSIISTLT

SEQ ID NO: 239 - DNA, 399 bp, Artificial Sequence
  L80F, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 240 - protein, 133 aa, Artificial Sequence
  L80F, C125S human IL-2 mutein
APTSSSTKKTQLQLEHLLLDLQMILNGINNYKNPKLTRMLTFKFYMPKKATELKHLQCLEEELKPLEEVLNLAQSKNFHFRPRDLISNINVIVLELKGSETTFMCEYADETATIVEFLNRWITFSQSIISTLT

SEQ ID NO: 241 - DNA, 399 bp, Artificial Sequence
  L80G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacggtagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 242 - protein, 133 aa, Artificial Sequence
  L80G, C125S human IL-2 mutein
APTSSSTKKTQLQLEHLLLDLQMILNGINNYKNPKLTRMLTFKFYMPKKATELKHLQCLEEELKPLEEVLNLAQSKNFHGRPRDLISNINVIVLELKGSETTFMCEYADETATIVEFLNRWITFSQSIISTLT

SEQ ID NO: 243 - DNA, 399 bp, Artificial Sequence
  L80K, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacaagagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 244 - protein, 133 aa, Artificial Sequence
  L80K, C125S human IL-2 mutein
APTSSSTKKTQLQLEHLLLDLQMILNGINNYKNPKLTRMLTFKFYMPKKATELKHLQCLEEELKPLEEVLNLAQSKNFHKRPRDLISNINVIVLELKGSETTFMCEYADETATIVEFLNRWITFSQSIISTLT

SEQ ID NO: 245 - DNA, 399 bp, Artificial Sequence
  L80N, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacaacagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 246 - protein, 133 aa, Artificial Sequence
  L80N, C125S human IL-2 mutein
APTSSSTKKTQLQLEHLLLDLQMILNGINNYKNPKLTRMLTFKFYMPKKATELKHLQCLEEELKPLEEVLNLAQSKNFHNRPRDLISNINVIVLELKGSETTFMCEYADETATIVEFLNRWITFSQSIISTLT

SEQ ID NO: 247 - DNA, 399 bp, Artificial Sequence
  L80R, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacagaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 248 - protein, 133 aa, Artificial Sequence
  L80R, C125S human IL-2 mutein
APTSSSTKKTQLQLEHLLLDLQMILNGINNYKNPKLTRMLTFKFYMPKKATELKHLQCLEEELKPLEEVLNLAQSKNFHRRPRDLISNINVIVLELKGSETTFMCEYADETATIVEFLNRWITFSQSIISTLT

SEQ ID NO: 249 - DNA, 399 bp, Artificial Sequence
  L80T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacacgagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 250 - protein, 133 aa, Artificial Sequence
  L80T, C125S human IL-2 mutein
APTSSSTKKTQLQLEHLLLDLQMILNGINNYKNPKLTRMLTFKFYMPKKATELKHLQCLEEELKPLEEVLNLAQSKNFHTRPRDLISNINVIVLELKGSETTFMCEYADETATIVEFLNRWITFSQSIISTLT

SEQ ID NO: 251 - DNA, 399 bp, Artificial Sequence
  L80V, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacgttagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 252 - protein, 133 aa, Artificial Sequence
  L80V, C125S human IL-2 mutein
APTSSSTKKTQLQLEHLLLDLQMILNGINNYKNPKLTRMLTFKFYMPKKATELKHLQCLEEELKPLEEVLNLAQSKNFHVRPRDLISNINVIVLELKGSETTFMCEYADETATIVEFLNRWITFSQSIISTLT

SEQ ID NO: 253 - DNA, 399 bp, Artificial Sequence
  L80W, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcactggagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 254 - protein, 133 aa, Artificial Sequence
  L80W, C125S human IL-2 mutein
APTSSSTKKTQLQLEHLLLDLQMILNGINNYKNPKLTRMLTFKFYMPKKATELKHLQCLEEELKPLEEVLNLAQSKNFHWRPRDLISNINVIVLELKGSETTFMCEYADETATIVEFLNRWITFSQSIISTLT

SEQ ID NO: 255 - DNA, 399 bp, Artificial Sequence
  L80Y, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcactacagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 256 - protein, 133 aa, Artificial Sequence
  L80Y, C125S human IL-2 mutein
APTSSSTKKTQLQLEHLLLDLQMILNGINNYKNPKLTRMLTFKFYMPKKATELKHLQCLEEELKPLEEVLNLAQSKNFHYRPRDLISNINVIVLELKGSETTFMCEYADETATIVEFLNRWITFSQSIISTLT

SEQ ID NO: 257 - DNA, 399 bp, Artificial Sequence
  R81E, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttagaacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 258 - protein, 133 aa, Artificial Sequence
  R81E, C125S human IL-2 mutein
APTSSSTKKTQLQLEHLLLDLQMILNGINNYKNPKLTRMLTFKFYMPKKATELKHLQCLEEELKPLEEVLNLAQSKNFHLEPRDLISNINVIVLELKGSETTFMCEYADETATIVEFLNRWITFSQSIISTLT

SEQ ID NO: 259 - DNA, 399 bp, Artificial Sequence
  R81K, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaaagcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 260 - protein, 133 aa, Artificial Sequence
  R81K, C125S human IL-2 mutein
APTSSSTKKTQLQLEHLLLDLQMILNGINNYKNPKLTRMLTFKFYMPKKATELKHLQCLEEELKPLEEVLNLAQSKNFHLKPRDLISNINVIVLELKGSETTFMCEYADETATIVEFLNRWITFSQSIISTLT

SEQ ID NO: 261 - DNA, 399 bp, Artificial Sequence
  R81L, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttattgcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 262 - protein, 133 aa, Artificial Sequence
  R81L, C125S human IL-2 mutein
APTSSSTKKTQLQLEHLLLDLQMILNGINNYKNPKLTRMLTFKFYMPKKATELKHLQCLEEELKPLEEVLNLAQSKNFHLLPRDLISNINVIVLELKGSETTFMCEYADETATIVEFLNRWITFSQSIISTLT

SEQ ID NO: 263 - DNA, 399 bp, Artificial Sequence
  R81M, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaatgcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 264 - protein, 133 aa, Artificial Sequence
  R81M, C125S human IL-2 mutein
APTSSSTKKTQLQLEHLLLDLQMILNGINNYKNPKLTRMLTFKFYMPKKATELKHLQCLEEELKPLEEVLNLAQSKNFHLMPRDLISNINVIVLELKGSETTFMCEYADETATIVEFLNRWITFSQSIISTLT

SEQ ID NO: 265 - DNA, 399 bp, Artificial Sequence
  R81N, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaaaccccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 266 - protein, 133 aa, Artificial Sequence
  R81N, C125S human IL-2 mutein
APTSSSTKKTQLQLEHLLLDLQMILNGINNYKNPKLTRMLTFKFYMPKKATELKHLQCLEEELKPLEEVLNLAQSKNFHLNPRDLISNINVIVLELKGSETTFMCEYADETATIVEFLNRWITFSQSIISTLT

SEQ ID NO: 267 - DNA, 399 bp, Artificial Sequence
  R81P, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaccacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 268 - protein, 133 aa, Artificial Sequence
  R81P, C125S human IL-2 mutein
APTSSSTKKTQLQLEHLLLDLQMILNGINNYKNPKLTRMLTFKFYMPKKATELKHLQCLEEELKPLEEVLNLAQSKNFHLPPRDLISNINVIVLELKGSETTFMCEYADETATIVEFLNRWITFSQSIISTLT

SEQ ID NO: 269 - DNA, 399 bp, Artificial Sequence
  R81T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaacgcccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 270 - protein, 133 aa, Artificial Sequence
  R81T, C125S human IL-2 mutein
APTSSSTKKTQLQLEHLLLDLQMILNGINNYKNPKLTRMLTFKFYMPKKATELKHLQCLEEELKPLEEVLNLAQSKNFHLTPRDLISNINVIVLELKGSETTFMCEYADETATIVEFLNRWITFSQSIISTLT

SEQ ID NO: 271 - DNA, 399 bp, Artificial Sequence
  D84R, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccaggagattaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 272 - protein, 133 aa, Artificial Sequence
  D84R, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRRLISNINVIVLELKGSETTFMCEYADETATIVEFLNRWITFSQSIISTLT

SEQ ID NO: 273 - DNA, 399 bp, Artificial Sequence
  S87T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcacgaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 274 - protein, 133 aa, Artificial Sequence
  S87T, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLITNINVIVLELKGSETTFMCEYADETATIVEFLNRWITFSQSIISTLT

SEQ ID NO: 275 - DNA, 399 bp, Artificial Sequence
  N88D, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcgatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 276 - protein, 133 aa, Artificial Sequence
  N88D, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 277 - DNA, 399 bp, Artificial Sequence
  N88H, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagccacatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 278 - protein, 133 aa, Artificial Sequence
  N88H, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISHINVIVLELKGSETTFMCEYADETATIVEFLNRWITFSQSIISTLT

SEQ ID NO: 279 - DNA, 399 bp, Artificial Sequence
  N88T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcacgatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 280 - protein, 133 aa, Artificial Sequence
  N88T, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISTINVIVLELKGSETTFMCEYADETATIVEFLNRWITFSQSIISTLT

SEQ ID NO: 281 - DNA, 399 bp, Artificial Sequence
  V91A, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgctatagttctggaactaaagggatctgaaacaacattcatgtgtgaatatgctgatgagacagcaaccattgtagaatttctgaacagatggattaccttttctcagagcatcatctcaacactgact

SEQ ID NO: 282 - protein, 133 aa, Artificial Sequence
  V91A, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINAIVLELKGSETTFMCEYADETATIVEFLNRWITFSQSIISTLT

SEQ ID NO: 283 - DNA, 399 bp, Artificial Sequence
  V91D, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgatatagttctggaactaaagggatctgaaacaacattcatgtgtgaatatgctgatgagacagcaaccattgtagaatttctgaacagatggattaccttttctcagagcatcatctcaacactgact

SEQ ID NO: 284 - protein, 133 aa, Artificial Sequence
  V91D, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINDIVLELKGSETTFMCEYADETATIVEFLNRWITFSQSIISTLT

SEQ ID NO: 285 - DNA, 399 bp, Artificial Sequence
  V91E, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgaaatagttctggaactaaagggatctgaaacaacattcatgtgtgaatatgctgatgagacagcaaccattgtagaatttctgaacagatggattaccttttctcagagcatcatctcaacactgact

SEQ ID NO: 286 - protein, 133 aa, Artificial Sequence
  V91E, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINEIVLELKGSETTFMCEYADETATIVEFLNRWITFSQSIISTLT

SEQ ID NO: 287 - DNA, 399 bp, Artificial Sequence
  V91F, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacttcatagttctggaactaaagggatctgaaacaacattcatgtgtgaatatgctgatgagacagcaaccattgtagaatttctgaacagatggattaccttttctcagagcatcatctcaacactgact

SEQ ID NO: 288 - protein, 133 aa, Artificial Sequence
  V91F, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINFIVLELKGSETTFMCEYADETATIVEFLNRWITFSQSIISTLT

SEQ ID NO: 289 - DNA, 399 bp, Artificial Sequence
  V91G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacggtatagttctggaactaaagggatctgaaacaacattcatgtgtgaatatgctgatgagacagcaaccattgtagaatttctgaacagatggattaccttttctcagagcatcatctcaacactgact

SEQ ID NO: 290 - protein, 133 aa, Artificial Sequence
  V91G, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINGIVLELKGSETTFMCEYADETATIVEFLNRWITFSQSIISTLT

SEQ ID NO: 291 - DNA, 399 bp, Artificial Sequence
  V91N, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacaacatagttctggaactaaagggatctgaaacaacattcatgtgtgaatatgctgatgagacagcaaccattgtagaatttctgaacagatggattaccttttctcagagcatcatctcaacactgact

SEQ ID NO: 292 - protein, 133 aa, Artificial Sequence
  V91N, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINNIVLELKGSETTFMCEYADETATIVEFLNRWITFSQSIISTLT

SEQ ID NO: 293 - DNA, 399 bp, Artificial Sequence
  V91Q, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaaccaaatagttctggaactaaagggatctgaaacaacattcatgtgtgaatatgctgatgagacagcaaccattgtagaatttctgaacagatggattaccttttctcagagcatcatctcaacactgact

SEQ ID NO: 294 - protein, 133 aa, Artificial Sequence
  V91Q, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINQIVLELKGSETTFMCEYADETATIVEFLNRWITFSQSIISTLT

SEQ ID NO: 295 - DNA, 399 bp, Artificial Sequence
  V91W, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaactggatagttctggaactaaagggatctgaaacaacattcatgtgtgaatatgctgatgagacagcaaccattgtagaatttctgaacagatggattaccttttctcagagcatcatctcaacactgact

SEQ ID NO: 296 - protein, 133 aa, Artificial Sequence
  V91W, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINWIVLELKGSETTFMCEYADETATIVEFLNRWITFSQSIISTLT

SEQ ID NO: 297 - DNA, 399 bp, Artificial Sequence
  L94A, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttgctgaactaaagggatctgaaacaacattcatgtgtgaatatgctgatgagacagcaaccattgtagaatttctgaacagatggattaccttttctcagagcatcatctcaacactgact

SEQ ID NO: 298 - protein, 133 aa, Artificial Sequence
  L94A, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVAELKGSETTFMCEYADETATIVEFLNRWITFSQSIISTLT

SEQ ID NO: 299 - DNA, 399 bp, Artificial Sequence
  L94I, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttatagaactaaagggatctgaaacaacattcatgtgtgaatatgctgatgagacagcaaccattgtagaatttctgaacagatggattaccttttctcagagcatcatctcaacactgact

SEQ ID NO: 300 - protein, 133 aa, Artificial Sequence
  L94I, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVIELKGSETTFMCEYADETATIVEFLNRWITFSQSIISTLT

SEQ ID NO: 301 - DNA, 399 bp, Artificial Sequence
  L94T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttacggaactaaagggatctgaaacaacattcatgtgtgaatatgctgatgagacagcaaccattgtagaatttctgaacagatggattaccttttctcagagcatcatctcaacactgact

SEQ ID NO: 302 - protein, 133 aa, Artificial Sequence
  L94T, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVTELKGSETTFMCEYADETATIVEFLNRWITFSQSIISTLT

SEQ ID NO: 303 - DNA, 399 bp, Artificial Sequence
  L94V, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttgttgaactaaagggatctgaaacaacattcatgtgtgaatatgctgatgagacagcaaccattgtagaatttctgaacagatggattaccttttctcagagcatcatctcaacactgact

SEQ ID NO: 304 - protein, 133 aa, Artificial Sequence
  L94V, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVVELKGSETTFMCEYADETATIVEFLNRWITFSQSIISTLT

SEQ ID NO: 305 - DNA, 399 bp, Artificial Sequence
  L94Y, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagtttacgaactaaagggatctgaaacaacattcatgtgtgaatatgctgatgagacagcaaccattgtagaatttctgaacagatggattaccttttctcagagcatcatctcaacactgact

SEQ ID NO: 306 - protein, 133 aa, Artificial Sequence
  L94Y, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVYELKGSETTFMCEYADETATIVEFLNRWITFSQSIISTLT

SEQ ID NO: 307 - DNA, 399 bp, Artificial Sequence
  E95D, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggacctaaagggatctgaaacaacattcatgtgtgaatatgctgatgagacagcaaccattgtagaatttctgaacagatggattaccttttctcagagcatcatctcaacactgact

SEQ ID NO: 308 - protein, 133 aa, Artificial Sequence
  E95D, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLDLKGSETTFMCEYADETATIVEFLNRWITFSQSIISTLT

SEQ ID NO: 309 - DNA, 399 bp, Artificial Sequence
  E95G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctgggtctaaagggatctgaaacaacattcatgtgtgaatatgctgatgagacagcaaccattgtagaatttctgaacagatggattaccttttctcagagcatcatctcaacactgact

SEQ ID NO: 310 - protein, 133 aa, Artificial Sequence
  E95G, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLGLKGSETTFMCEYADETATIVEFLNRWITFSQSIISTLT

SEQ ID NO: 311 - DNA, 399 bp, Artificial Sequence
  E95M, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctgatgctaaagggatctgaaacaacattcatgtgtgaatatgctgatgagacagcaaccattgtagaatttctgaacagatggattaccttttctcagagcatcatctcaacactgact

SEQ ID NO: 312 - protein, 133 aa, Artificial Sequence
  E95M, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLMLKGSETTFMCEYADETATIVEFLNRWITFSQSIISTLT

SEQ ID NO: 313 - DNA, 399 bp, Artificial Sequence
  T102S, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaagtttcatgtgtgaatatgctgatgagacagcaaccattgtagaatttctgaacagatggattaccttttctcagagcatcatctcaacactgact

SEQ ID NO: 314 - protein, 133 aa, Artificial Sequence
  T102S, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETSFMCEYADETATIVEFLNRWITFSQSIISTLT

SEQ ID NO: 315 - DNA, 399 bp, Artificial Sequence
  T102V, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacagttttcatgtgtgaatatgctgatgagacagcaaccattgtagaatttctgaacagatggattaccttttctcagagcatcatctcaacactgact

SEQ ID NO: 316 - protein, 133 aa, Artificial Sequence
  T102V, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETVFMCEYADETATIVEFLNRWITFSQSIISTLT

SEQ ID NO: 317 - DNA, 399 bp, Artificial Sequence
  M104G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcggttgtgaatatgctgatgagacagcaaccattgtagaatttctgaacagatggattaccttttctcagagcatcatctcaacactgact

SEQ ID NO: 318 - protein, 133 aa, Artificial Sequence
  M104G, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFGCEYADETATIVEFLNRWITFSQSIISTLT

SEQ ID NO: 319 - DNA, 399 bp, Artificial Sequence
  E106K, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtaagtatgctgatgagacagcaaccattgtagaatttctgaacagatggattaccttttctcagagcatcatctcaacactgact

SEQ ID NO: 320 - protein, 133 aa, Artificial Sequence
  E106K, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCKYADETATIVEFLNRWITFSQSIISTLT

SEQ ID NO: 321 - DNA, 399 bp, Artificial Sequence
  Y107H, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaacacgctgatgagacagcaaccattgtagaatttctgaacagatggattaccttttctcagagcatcatctcaacactgact

SEQ ID NO: 322 - protein, 133 aa, Artificial Sequence
  Y107H, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEHADETATIVEFLNRWITFSQSIISTLT

SEQ ID NO: 323 - DNA, 399 bp, Artificial Sequence
  Y107K, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaaaaggctgatgagacagcaaccattgtagaatttctgaacagatggattaccttttctcagagcatcatctcaacactgact

SEQ ID NO: 324 - protein, 133 aa, Artificial Sequence
  Y107K, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEKADETATIVEFLNRWITFSQSIISTLT

SEQ ID NO: 325 - DNA, 399 bp, Artificial Sequence
  Y107L, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaattggctgatgagacagcaaccattgtagaatttctgaacagatggattaccttttctcagagcatcatctcaacactgact

SEQ ID NO: 326 - protein, 133 aa, Artificial Sequence
  Y107L, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCELADETATIVEFLNRWITFSQSIISTLT

SEQ ID NO: 327 - DNA, 399 bp, Artificial Sequence
  Y107Q, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaacaagctgatgagacagcaaccattgtagaatttctgaacagatggattaccttttctcagagcatcatctcaacactgact

SEQ ID NO: 328 - protein, 133 aa, Artificial Sequence
  Y107Q, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEQADETATIVEFLNRWITFSQSIISTLT

SEQ ID NO: 329 - DNA, 399 bp, Artificial Sequence
  Y107R, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaaagagctgatgagacagcaaccattgtagaatttctgaacagatggattaccttttctcagagcatcatctcaacactgact

SEQ ID NO: 330 - protein, 133 aa, Artificial Sequence
  Y107R, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCERADETATIVEFLNRWITFSQSIISTLT

SEQ ID NO: 331 - DNA, 399 bp, Artificial Sequence
  Y107T, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaaacggctgatgagacagcaaccattgtagaatttctgaacagatggattaccttttctcagagcatcatctcaacactgact

SEQ ID NO: 332 - protein, 133 aa, Artificial Sequence
  Y107T, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCETADETATIVEFLNRWITFSQSIISTLT

SEQ ID NO: 333 - DNA, 399 bp, Artificial Sequence
  E116G, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtaggttttctgaacagatggattaccttttctcagagcatcatctcaacactgact

SEQ ID NO: 334 - protein, 133 aa, Artificial Sequence
  E116G, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVGFLNRWITFSQSIISTLT

SEQ ID NO: 335 - DNA, 399 bp, Artificial Sequence
  N119Q, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgcaaagatggattaccttttctcagagcatcatctcaacactgact

SEQ ID NO: 336 - protein, 133 aa, Artificial Sequence
  N119Q, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLQRWITFSQSIISTLT

SEQ ID NO: 337 - DNA, 399 bp, Artificial Sequence
  T123C, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggatttgtttttctcagagcatcatctcaacactgact

SEQ ID NO: 338 - protein, 133 aa, Artificial Sequence
  T123C, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWICFSQSIISTLT

SEQ ID NO: 339 - DNA, 399 bp, Artificial Sequence
  T123S, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattagtttttctcagagcatcatctcaacactgact

SEQ ID NO: 340 - protein, 133 aa, Artificial Sequence
  T123S, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWISFSQSIISTLT

SEQ ID NO: 341 - DNA, 399 bp, Artificial Sequence
  Q126I, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctataagcatcatctcaacactgact

SEQ ID NO: 342 - protein, 133 aa, Artificial Sequence
  Q126I, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSISIISTLT

SEQ ID NO: 343 - DNA, 399 bp, Artificial Sequence
  Q126V, C125S human IL-2 mutein
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctgttagcatcatctcaacactgact

SEQ ID NO: 344 - protein, 133 aa, Artificial Sequence
  Q126V, C125S human IL-2 mutein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSVSIISTLT

SEQ ID NO: 345 - DNA, 399 bp, Artificial Sequence
  E61R, C125S human IL-2 mutein with E61R codon optimized for E. coli expression
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaacgtgaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgact

SEQ ID NO: 346 - DNA, 399 bp, Artificial Sequence
  Y107R, C125S human IL-2 mutein with Y107R codon optimized for E. coli expression
  CDS (1)...(399)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaacgtgctgatgagacagcaaccattgtagaatttctgaacagatggattaccttttctcagagcatcatctcaacactgact